(19) United States
(12) Patent Application Publication
(10) Pub. No.: US 2015/0265666 A1
(43) Pub. Date: Sep. 24, 2015

(54) BOTANICAL ANTIMICROBIAL COMPOSITIONS

(71) Applicant: THE TRUSTEES OF COLUMBIA UNIVERSITY IN THE CITY OF NEW YORK, New York, NY (US)

(72) Inventors: Shanta M. Modak, River Edge, NJ (US); Santoshkumar Dongre, New York, NY (US); Jaishree Vaijanathappa, New York, NY (US); Arnab Kumar Ghosh, Jersey City, NJ (US)

(73) Assignee: THE TRUSTEES OF COLUMBIA UNIVERSITY IN THE CITY OF NEW YORK, New York, NY (US)

(21) Appl. No.: 14/735,051

(22) Filed: Jun. 9, 2015

Related U.S. Application Data
(63) Continuation of application No. PCT/US13/71731, filed on Nov. 25, 2013.
(60) Provisional application No. 61/736,932, filed on Dec. 13, 2012.
(60) Provisional application No. 61/831,510, filed on Jun. 5, 2013.

(51) Int. Cl. A61K 36/53; A61K 33/40; A61K 36/28; A61K 36/185; A61K 31/155; A61K 36/71; A61K 45/06; A61K 36/752; A61K 36/898; A61K 33/20; A61K 36/81

(52) U.S. Cl. CPC A61K 36/53; A61K 33/20; A61K 33/40; A61K 36/28; A61K 36/185; A61K 36/81; A61K 36/71; A61K 45/06; A61K 36/752; A61K 36/898; A61K 31/155

(57) ABSTRACT

Disclosed herein are antimicrobial compositions comprising mixtures of botanical extracts, synthetic antimicrobial agents and essential oils which do not rely solely upon alcohol to produce their antimicrobial effects.

### PRIORITY CLAIM

This application is a continuation of International Patent Application Serial No. PCT/US2013/071731, filed Nov. 25, 2013, which claims priority to U.S. Provisional Application Ser. No. 61/736,932, filed Dec. 13, 2012, and U.S. Provisional Application Ser. No. 61/831,510, filed Jun. 5, 2013, to each of which priority is claimed, and the contents of each of which are incorporated by reference in their entireties.

### 1. INTRODUCTION

Disclosed here are antimicrobial compositions comprising mixtures of botanical extracts, synthetic antimicrobial agents and essential oils which do not rely solely upon alcohol to produce their antimicrobial effects.

### 2. BACKGROUND

The Centers for Disease Control and Prevention (CDC) have issued guidelines recommending the use of alcohol-based products (60-70% alcohol) for skin disinfection to reduce the transmission of pathogenic organisms to patients and personnel in health care settings. However, alcohol-based hand sanitizers, although effective in rapidly killing germs, disrupt the skin's moisture and pH balance by stripping away the natural oils, leaving the skin dry, cracked, and more susceptible to infections. Further, alcohol based sanitizers with 60-70% alcohol are highly flammable and can be misused with toxic consequences. According to a 2007 report published by the American Association of Poison Control Canter's, in 2006 there were approximately 12000 incidents of alcohol-poisoning directly attributed to alcohol based hand sanitizers.

There is a continuing desire for an antimicrobial composition which has a sufficiently low amount of alcohol to avoid the skin damage, flammability, and abuse potential of alcohol-dependent formulations (or which lacks alcohol altogether).

### 3. SUMMARY

Described herein are antimicrobial compositions comprising mixtures of botanical extracts, synthetic antimicrobial agents and essential oils which do not rely solely upon alcohol to produce their antimicrobial effects.

In certain embodiments, an antimicrobial composition comprises (a) one or more essential oil or constituent thereof selected from thyme oil, thymol, fennel oil (e.g., fennel seed oil), vanilla oil and basil oil at a concentration of between about 0.01 and 0.5 percent weight/weight (“% w/w”); and (b) one or more synthetic antimicrobial selected from (i) biguanides, such as, for example, chlorhexidine free base or chlorhexidine salt (e.g. chlorhexidine gluconate or chlorhexidine acetate), polyhexamethylene biguanide (PHMB), polyaminipropyl biguanide (PAPB), and combinations thereof, at a concentration of between about 0.01 and 0.5% w/w; and (ii) quaternary ammonium compound, such as, for example, benzalkonium chloride (BAC), benzethonium chloride (BZT), cetyl pyridinium chloride, alkyl dimethyl benzyl ammonium chloride, dimethyl benzyl ammonium chloride, delmopinol hydrochloride, and combinations thereof, at a concentration of between about 0.02 and 0.5% w/w.

In certain non-limiting embodiments, an antimicrobial composition comprises (a) one or more plant extract, which may be vanilla extract, lemon extract, orange extract, grapefruit extract, grapefruit seed extract, Bacopa monniera (a.k.a. Brahmi) extract, honeysuckle extract, Echinacea (which may be Echinacea purpurea, Echinacea angustifolia or Echinacea pallida) extract, goldenseal (Hydrastis canadensis) extract, Portulaca oleracea extract, witchhazel extract, rosemary extract, stevia extract, tobacco extract, wasabi extract and/or willow bark extract, at a concentration of between about 0.1 and 1.0% w/w or between about 0.3 and 0.6% w/w; and (b) one or more synthetic antimicrobial, which may be a biguanide such as chlorhexidine free base or chlorhexidine salt (e.g. chlorhexidine gluconate or chlorhexidine acetate) or polyhexamethylene biguanide, a quaternary ammonium compound such as benzalkonium chloride (BAC), benzethonium chloride (BZT), or cetylpyridinium chloride (CPC), or delmopinol hydrochloride, at a concentration of between about 0.05 and 0.5% w/w; where said composition may comprise less than 21% w/w or less than 10% w/w or less than 5% w/w or less than 2% w/w or less than 1% w/w or less than 0.5% w/w or less than 0.01% w/w, or essentially no, alcohol; and/or no benzyl alcohol, and/or no ethanol, and/or no isopropyl alcohol. Said antimicrobial composition may optionally further comprise one or more of (i) an alkanediol which may be propanediol, pentanediol, hexanediol, heptanediol, octanediol, nonanediol or decandediol at a concentration between about 0 and 2.0% w/w or between about 0.01 and 2.0% w/w or between about 0.3 and 2.0% w/w; (ii) a solubulizer which may be a plant-based mild solubilizing non ionic surfactant, for example caprilyl capryl glucoside, decyl glucoside, or a combination thereof; (iii) a plant-based organic acid which may be lactic acid, citric acid, benzoic acid or a combination thereof (e.g., at a concentration of between about 0.1-0.5% w/w); (iv) an essential oil or constituent thereof which may be thymol, rosemary oil, lemongrass oil, cinnamon oil, pine oil, clove bud oil, basil oil, cedarwood oil, or a combination thereof (e.g. at a concentration of between about 0.05 and 0.5% w/w or between about 0.05 and 0.2% w/w or between about 0.1 and 0.2% w/w); (v) a botanical anti-irritant which may be aloe gel/extract (e.g. at a concentration between about 0.1-50% w/w); aloe leaf juice (e.g. at a concentration of between about 0.5-2.05% w/w); bisabolol (e.g. at a concentration between about 0.05-2.0% w/w); and/or an oat product such as beta glucan, oat kernel extract, or oat flour (e.g. at a concentration between about 0.2 and 3.0% w/w); (vi) an anti-inflammatory agent which may be calendula oil, rosemary oil, a curcumin compound (e.g. curcumin, desmethoxycurcumin, bis-desmethoxycurcumin, tetrahydrocurcuminoids), pomegranate oil, clove oil, Echinacea purpurea extract, resveratrol (e.g. at a concentration between about 0.1 and 0.5% w/w), portulaca extract and/or witchhazel extract (e.g. at a concentration up to about 2.0% w/w); (vii) a solvent which may be a solubilizer-containing mixture of PEG-40 hydrogenated castor oil, tridecath 9 and water (e.g. at a concentration between about 0.5 and 5.0% w/w), phenyl ethanol (e.g. at a concentration between about 0.3 and 1.0% w/w), and alcohol (e.g. at a concentration up to about 5% w/w or up to about 10% w/w or up to about 15% w/w or up to about 20% w/w or up to about 25% w/w); and/or (viii) an additional antimicrobial agent/anti biofilm agents which may be sodium pyrithion, zinc pyrithion, a silver salt such as silver nitrate, silver sulfadiazine, povidone iodine, xylitol, Germall plus (diazolidinyl urea and iodopropynyl butylcarbamate solubilized in propylene glycol), hypochlorite, hydrogen peroxide, urea hydrogen peroxide, sodium hypochlorite and/or triclosan.

In certain non-limiting embodiments, the antimicrobial composition described herein comprises: (i) a plant extract selected from the group consisting of vanilla extract, lemon extract, citrus extract, grapefruit seed extract and combinations thereof at a concentration of between about 0.01 and 5% w/w, or between about 0.05 and 1% w/w, or between about 0.1 and 0.6% w/w;

(ii) polyhexamethylene biguanide (PHMB), polyaminopropyl biguanide (PAPB) or combinations thereof at a concentration of between about 0.001 and 5% w/w, or between about 0.005 and 1% w/w, or between about 0.01 and 0.3% w/w; and

(iii) one or more essential oil or active ingredient thereof selected from the group consisting of lemongrass oil, lemon oil, fennel seed oil, orange oil, basil oil, cinnamon oil, thyme oil, clove oil, pine oil, cedar wood oil, thymol, farnesol, bisabolol, cinnamaldehyde, cinnamylacetic ester, cinnamic acid, ethyl cinnamate or combinations thereof, at a concentration of between about 0.001 and 5% w/w, or between about 0.005 and 1% w/w, or between about 0.01 and 0.5% w/w.

In certain embodiments, the PHMB is comprised in a Cosmocil composition. In certain embodiments, the Cosmocil composition comprises PHMB at a concentration of about 20% w/w.

In certain embodiments, the composition comprises vanilla extract, lemon extract or combinations thereof, and does not comprise any other citrus extract.

In certain non-limiting embodiments, the antimicrobial composition described herein further comprises: (iv) one or more alkanediol selected from the group consisting of propanediol, pentanediol, hexanediol, heptanediol, octanediol, decanediol, nonanediol and combinations thereof, at a concentration of between about 0 and 10% w/w, or between about 0 and 10% w/w, or between about 0 and 5% w/w, or between about 0.01 and 0.5% w/w;

(v) an organic acid selected from the group consisting of benzoic acid, lactic acid, citric acid and combinations thereof, at a concentration of between about 0.005 and 5% w/w, or between about 0.01 and 1% w/w, or between about 0.05 and 0.5% w/w;

(vi) a solvents which enhances the solubility of the ingredients of the composition, selected from the group consisting of alcohol (between about 0 and 80% w/w), phenyl ethanol (between about 0 and 1.0% w/w), a mixture of PEG-40 hydrogenated castor oil, Trideceth-9, propylene glycol and water (between about 0 and 2% w/w), a mixture of PEG-40 hydrogenated castor oil, Trideceth-9 and water (between about 0 and 2% w/w), caprylyl capryl glucoside (Plantasol) (between about 0 and 2% w/w), and combinations thereof;

(vii) one or more anti-irritant selected from the group consisting of zinc salicylate, basil oil, brahmi extract, red sandal wood extract, curcumin compound, aloe, bisabolol, rosemary oil, chalmogra oil, oat powder and/or extract, beta glucan, chitosan, calendula oil/extract, portulaca extract, witch hazel extract, pomegranate oil, at a concentration of between about 0.01 and 10% w/w, or between about 0.05 and 5% w/w, or between about 0.1 and 2% w/w.

In certain non-limiting embodiments, the antimicrobial composition described herein comprises: (i) a plant extract selected from the group consisting of vanilla extract, lemon extract and combinations thereof at a concentration of between about 0.1 and 1% w/w; (ii) one or more synthetic antimicrobial selected from the group consisting of polyhexamethylene biguanide (PHMB), polyaminopropyl biguanide (PAPB), and combinations thereof, at a concentration of between about 0.001 and 0.5% w/w; and (iii) one or more essential oil or constituent thereof selected from the group consisting of thyme oil, thymol, cinnamon oil, cinnamaldehyde, cinnamylacetic ester, cinnamic acid, ethyl cinnamate, fennel oil, basil oil, lemongrass oil, lemon oil, orange oil, clove oil, pine oil, cedarwood oil, rosemary oil, and combinations thereof, at a concentration of between about 0.01 and 0.5% w/w.

In certain non-limiting embodiments, the antimicrobial composition described herein comprises: (i) a plant extract selected from the group consisting of vanilla extract, lemon extract and combinations thereof at a concentration of between about 0.1 and 0.6% w/w; (ii) polyhexamethylene biguanide (PHMB) at a concentration of between about 0.01 and 0.3% w/w; and (iii) one or more essential oil or active ingredient thereof selected from the group consisting of thyme oil, cinnamon oil, thymol, cinnamaldehyde, cinnamylacetic ester, cinnamic acid, ethyl cinnamate or combinations thereof, at a concentration of between about 0.01 and 0.5% w/w.

In certain non-limiting embodiments, the antimicrobial composition described herein comprises essentially no benzyl alcohol.

Antimicrobial compositions disclosed herein may be used as preservative additives to other products (e.g. to oral care, personal care, household, veterinary or industrial products) or may themselves serve, with additional ingredients as known in the art, as personal care, household, veterinary or industrial products. Non-limiting examples include skin sanitizer/disinfectant (e.g. hand sanitizer, body wash) in the form of a lotion, foam, cream or wipe; surface sanitizer/disinfectant in the form of a liquid or wipe, veterinary sanitizer/disinfectant in the form of a liquid, foam, lotion, cream or wipe; oral care rinse; oral care wipe; oral care gel; oral care toothpaste; topical anti-itch treatment, anti-inflammatory gel/cream, wound care formulations which may be a lotion, cream, spray, wipe, or wound covering; or animal teat dip or wipe (e.g. cow teat dip or wipe).

As used herein, the term “chlorhexidine free base” means the basic form of chlorhexidine.

In certain embodiments, the weight percentages of the ingredients of the formulations described herein can be adjusted to provide for concentrated formulations that can be diluted prior to use. For example, the ingredients of the formulations described herein can be adjusted to concentrations of between about 1 and 25 times (1-25×) the % w/w identified in the formulations herein For example, the % w/w of the ingredients of the formulations described herein can be adjusted to concentrations of 1×, 2×, 3×, 4×, 5×, 6×, 7×, 8×, 9×, 10×, 15×, 20× or 25×, and values in between.

In the formulations disclosed herein, where the weight percentages of ingredients listed do not add up to 100 percent, the additional components may include components known in the art for an intended use, for example in personal care products, oral formulations, foods, beverages, and as topical or surface disinfectants. As non-limiting examples, the components may comprise one or more of water or other solvent, thickening agent(s), emollient(s), food product, etc., as the case may be.

### 4. DETAILED DESCRIPTION

Ranges specified herein include their recited limits; for example, a range of between 2 and 4 includes 2 and 4.

For clarity but not by way of limitation, the detailed description is divided into the following subsections,

(i) active agents;

(ii) oral formulations

(iii) topical disinfectants; and

(iv) surface disinfectants.

### 4.1 Active Agents

In certain non-limiting embodiments, an antimicrobial composition is provided comprising mixtures of botanical extracts, synthetic antimicrobial agents and essential oils which do not rely solely upon alcohol to produce their antimicrobial effects.

In certain embodiments, an antimicrobial composition comprises:

(a) one or more essential oil or constituent thereof selected from thyme oil, thymol, fennel oil (e.g., fennel seed oil), vanilla oil and basil oil at a concentration of between about 0.001 and about 1 percent weight/weight (“% w/w”), between about 0.01 and about 0.8% w/w, between about 0.1 and about 0.6% w/w, or between about 0.2 and about 0.5% w/w. In certain embodiments, the one or more essential oil or constituent thereof is at a concentration of between about 0.01 and about 0.5% w/w;

(b) one or more synthetic antimicrobial selected from (i) biguanides, such as, for example, chlorhexidine free base or chlorhexidine salt (e.g. chlorhexidine gluconate or chlorhexidine acetate), polyhexamethylene biguanide (PHMB), polyaminipropyl biguanide (PAPS), and combinations thereof, at a concentration of between about 0.001 and about 6% w/w, between about 0.001 and about 1% w/w, between about 0.001 and about 0.5% w/w, between about 0.01 and about 0.8% w/w, between about 0.01 and about 0.5% w/w, between about 0.1 and about 0.6% w/w, or between about 0.2 and about 0.5% w/w; (ii) quaternary ammonium compound, such as, for example, benzalkonium chloride (BAC), benzethonium chloride (BZT), cetyl pyridinium chloride, alkyl dimethyl benzyl ammonium chloride, dimethyl benzyl ammonium chloride, delmopinol hydrochloride, and combinations thereof, at a concentration of between about 0.001 and about 6% w/w, between about 0.001 and about 1% w/w, between about 0.001 and about 0.5% w/w, between about 0.01 and about 0.8% w/w, between about 0.02 and about 0.5% w/w, between about 0.1 and about 0.6% w/w, or between about 0.2 and about 0.5% w/w, and (iii) a hypochlorite, hydrogen peroxide, urea hydrogen peroxide, and combinations thereof, at a concentration of between about 0.001 and about 6% w/w, between about 0.001 and about 1% w/w, between about 0.001 and about 0.5% w/w, between about 0.01 and about 0.8% w/w, between about 0.02 and about 0.5% yaw, between about 0.1 and about 0.6% w/w, or between about 0.2 and about 0.5% w/w.

As used herein, the term “chlorhexidine free base” means the basic form of chlorhexidine.

In certain embodiments, the antimicrobial composition comprising one or more essential oil or constituent thereof and one or more synthetic antimicrobial comprises essentially no benzyl alcohol.

The antimicrobial composition comprising one or more essential oil or constituent thereof and one or more synthetic antimicrobial may optionally further comprise one or more of the following:

(c) one or more essential oil or constituent thereof selected from lemon grass oil, lemon oil, orange oil, basil oil, cinnamon oil, rosemary oil, peppermint oil, spearmint oil, eucalyptus oil, clove oil, pine oil, cedar wood oil, farnesol, and bisabolol at a concentration of between 0.01 and 0.7% w/w;

(d) one or more alkanediol selected from propanediol (e.g., Zemea®), pentanediol, hexanediol, heptanediol, octanediol, decanediol and nonanediol, at a concentration of between about 0.3 and 5.0% w/w;

(e) one or more organic acid selected from benzoic acid, lactic acid and citric acid, at a concentration of between about 0.05 and 0.5% w/w;

(f) one or more botanical extract such as a citrus extract, lemon extract, orange extract, grapefruit seed extract, grapefruit extract, and vanilla extract, at a concentration of between about 0 and about 5% w/w, between about 01 and about 1% w/w, or between about 0.1 and 0.6% w/w;

(g) one or more solvents and/or solubilizer selected from alcohol (e.g., ethyl alcohol or ethanol) (0-80% w/w), phenyl ethanol (0-1.0% w/w, or 0.3-1.0% w/w, or 25% w/w or less, or 20% w/w or less, or 15% w/w or less, or 10% w/w or less, or 5% w/w or less), PEG-40 hydrogenated Castor oil, trideceth-9, propylene glycol, water (0-2.0% w/w), caprylyl capryl glucoside (Plantasol) (0-2.0% w/w);

(h) one or more antimicrobial agent selected from sodium pyrithion, zinc pyrithion, silver salt (e.g., silver nitrate, and/or silver sulfadiazine), povidone iodine, Germall plus (diazolidinyl urea and iodopropynyl butylcarbamate solubilized in propylene glycol), and triclosan.

(i) one or more anti-irritant selected from brahmi extracts, red sandal wood extracts, curcumin compounds, aloe, bisabolol, rosemary oil, chalmogra oil, oat powder, oat extract, beta glucan, chitosan, calendula oil, portulaca extract, witch hazel extract, and pomegranate oil, at a concentration of between about 0.1 and 2.0% w/w.

In certain non-limiting embodiments, an antimicrobial composition is provided comprising (a) one or more plant/fruit extract which may be a citrus extract such as a lemon extract, orange extract, grapefruit seed extract or grapefruit extract or vanilla extract or a combination thereof (e.g. at a concentration between about 0.15 and 0.6% w/w); (b) one or more synthetic antimicrobial which may be a quaternary ammonium compound such as benzalkonium chloride, benzethonium chloride, cetyl pyrinidium chloride, alkyl dimethyl benzyl ammonium chloride or dimethyl benzyl ammonium chloride, a biguanide such as chlorhexidine free base or chlorhexidine salt (e.g. chlorhexidine gluconate or chlorhexidine acetate) or polyhexamethylene biguanide, or a combination thereof (e.g. at a concentration between about 0.05 and 0.5% w/w); (c) one or more essential oil or active ingredient thereof which may be thymol, rosemary oil, lemongrass oil, cinnamon oil, or a combination thereof (e.g. at a concentration of between about 0.05 and 0.5% w/w); (d) one or more plant-based mild solubilizing nonionic surfactant which may be caprilyl capryl glucoside, decyl glucoside, or a combination thereof (e.g. at a concentration of between about 0.3 and 10.0% w/w); (e) one or more plant-based organic acid which may be lactic acid, citric acid, benzoic acid, or a combination thereof (e.g. at a concentration between about 0.1 and 0.5% w/w); (f) one or more botanical anti-irritant which may be aloe gel/extract or leaf juice (e.g. at a concentration between about 0.1 and 2.0% w/w), bisabolol (e.g. at a concentration between about 0.05 and 1.0% w/w); and (g) one or more anti-inflammatory agent which may be calendula oil, rosemary oil, a curcumin compounds, pomegranate oil, Echinacea purpurea extract, resveratrol or a combination thereof (e.g. at a concentration of between about 0.1 and 0.5% w/w). Additional optional components of such compositions may include (i) one or more additional solvent such as a solubilizer-containing mixture of PEG-40 Hydrogenated castor oil, Tridecath 9 and water (e.g. at a concentration of between about 0.5 and 5.0% w/w), phenyl ethanol (e.g. at a concentration of between about 0.3 and 1.0% w/w); (ii) an alkanediol which may be pentanediol, hexanediol, heptanediol, octanediol, nonanediol, or decandediol (e.g. at a concentration of between about 0.3 and 1.05% w/w); (iii) an emollient which may be 1,3-propanediol, butylene glycol, propylene glycol, dipropylene glycol, glycerine, diglycerin, 2-methyl-1,3-propanediol (Solidol MP-D), or ethoxy diglycol (e.g. at a concentration of between about 0.5 and 5.0% w/w); (iv) a gelling agent which may be hydroxypropylmethyl cellulose, hydroxy propyl methyl cellulose stearoxy ether (Sangelose), chitosan, pyrrolidone carboxylate (Kytamer) (e.g. at a concentration between about 0.01 and 1.0% w/w); and/or (v) a silicone compound or product which may be isododecane and isononanoate (clearocast 550), dimethicone copolyol (Dow coming 2501 cosmetic wax), or silicone polyether copolymer (Dow coming 193 C fluid).

In certain non-limiting embodiments, the antimicrobial composition described herein comprises: (i) a plant extract selected from the group consisting of vanilla extract, lemon extract, citrus extract, grapefruit seed extract and combinations thereof at a concentration of between about 0.05 and 1% w/w, or between about 0.1 and 0.6% w/w;

(ii) polyhexamethylene biguanide (PHMB), polyaminopropyl biguanide (PAPB) or combinations thereof at a concentration of between about 0.001 and 5% w/w, or between about 0.005 and 1% w/w, or between about 0.01 and 0.3% w/w; and

(iii) one or more essential oil or active ingredient thereof selected from the group consisting of lemongrass oil, lemon oil, fennel seed oil, orange oil, basil oil, cinnamon oil, thyme oil, clove oil, pine oil, cedar wood oil, thymol, farnesol, bisabolol, cinnamaldehyde, cinnamylacetic ester, cinnamic acid, ethyl cinnamate or combinations thereof, at a concentration of between about 0.005 and 1% w/w, or between about 0.01 and 0.5% w/w.

In certain embodiments, the composition comprises vanilla extract, lemon extract or combinations thereof, and does not comprise any other citrus extract.

In certain non-limiting embodiments, the antimicrobial composition described herein further comprises: (iv) one or more alkanediol selected from the group consisting of propanediol, pentanediol, hexanediol, heptanediol, octanediol, decanediol, nonanediol and combinations thereof, at a concentration of between about 0 and 10% w/w, or between about 0 and 5% w/w, or between about 0.01 and 0.5% w/w;

(v) an organic acid selected from the group consisting of benzoic acid, lactic acid, citric acid and combinations thereof, at a concentration of between about 0.01 and 1% w/w, or between about 0.05 and 0.5% w/w;

(vi) a solvents which enhances the solubility of the ingredients of the composition, selected from the group consisting of alcohol (between about 0 and 80% w/w), phenyl ethanol (between about 0 and 1.0% w/w), a mixture of PEG-40 hydrogenated castor oil, Trideceth-9, propylene glycol and water (between about 0 and 2% w/w), a mixture of PEG-40 hydrogenated castor oil, Trideceth-9 and water (between about 0 and 2% w/w), caprylyl capryl glucoside (Plantasol) (between about 0 and 2% w/w), and combinations thereof;

(vii) one or more anti-irritant selected from the group consisting of zinc salicylate, basil oil, brahmi extract, red sandal wood extract, curcumin compound, aloe, bisabolol, rosemary oil, chalmogra oil, oat powder and/or extract, beta glucan, chitosan, calendula oil, portulaca extract, witch hazel extract, pomegranate oil, at a concentration of between about 0.05 and 5% w/w, or between about 0.1 and 2% w/w.

In certain non-limiting embodiments, the antimicrobial composition described herein comprises: (i) a plant extract selected from the group consisting of vanilla extract, lemon extract and combinations thereof at a concentration of between about 0.1 and 0.6% w/w; (ii) polyhexamethylene biguanide (PHMB) at a concentration of between about 0.01 and 0.3% w/w; and (iii) one or more essential oil or active ingredient thereof selected from the group consisting of thyme oil, cinnamon oil, thymol, cinnamaldehyde, cinnamylacetic ester, cinnamic acid, ethyl cinnamate or combinations thereof, at a concentration of between about 0.01 and 0.5% w/w.

In certain non-limiting embodiments, the antimicrobial composition described herein comprises:

(i) one or more plant extract selected from vanilla extract, lemon extract, orange extract, grapefruit seed extract, Bacopa monniera extract, honeysuckle extract, Echinacea extract, goldenseal (Hydrastis canadensis) extract, Portulaca oleracea extract, witchhazel extract, rosemary extract, stevia extract, tobacco extract, wasabi extract and/or willow bark extract, at a concentration of between about 0 and about 5.0% w/w, or between about 0.1 and about 1.0% w/w;

(ii) one or more synthetic antimicrobial selected from a biguanide such as chlorhexidine free base or chlorhexidine salt, polyhexamethylene biguanide, polyaminopropyl biguanide, and combinations thereof; a quaternary ammonium compound such as benzalkonium chloride (BAC), delmopinol hydrochloride, benzethonium chloride (BZT), cetylpyridinium chloride (CPC), dimethyl benzyl ammonium chloride, and combinations thereof; a hypochlorite, hydrogen peroxide, and combinations thereof; at a concentration of between about 0.001 and about 6.0% w/w and between about 0.001 and about 0.5% w/w;

(iii) essential oil or constituent thereof selected from thyme oil, thymol, cinnamon oil, cinnamaldehyde, cinnamylacetic ester, cinnamic acid, ethyl cinnamate, rosemary oil, lemongrass oil, lemon oil, orange oil, cinnamon oil, fennel oil, vanilla oil, basil oil, clove oil, farnesol, bisabalol, and combinations thereof, at a concentration of between about 0.001 and about 5% w/w or between about 0.01 and about 0.5% w/w; and

(iv) essentially no benzyl alcohol.

In certain specific embodiments, a pure vanilla extract is incorporated into an antimicrobial composition. Pure Vanilla Extract may be manufactured in accordance with the U.S. standard of identify. Vanilla is the only flavor that has a standard of identity. This means, by law vanilla extract can only contain certain ingredients to be labeled as pure. The standard of identity requires 13.35 ounces of cured, cut vanilla beans (with a moisture content not greater than 25.00%) be used per gallon of finished extract. The extract must contain a minimum 35.00% alcohol by volume.

In certain specific embodiments, a pure lemon extract is incorporated into an antimicrobial composition. Pure Lemon Extract may be prepared by alcohol/water extraction of fresh lemon. The extract major ingredient is lemon oil.

In certain non-limiting embodiments, the pH of the compositions described herein are at a pH of between about 3 and 6, or between about 3.5 and 5.5 or between about 4 and 5. Any method known in the art may be used to adjust the pH of a composition referred to herein. In specific non-limiting embodiments, sodium hydroxide or triethanolamine may be added to increase the pH. In specific non-limiting embodiments, organic acids such as lactic acid, citric acid, and/or benzoic acid may be added to decrease the pH.

### 4.2 Oral Formulations

In certain embodiments, the compositions described herein are oral care compositions for use in reducing the number and/or growth of microbes in the mouth of a subject. Also provided is a method of reducing the number and/or growth of microbes in the mouth of a subject comprising applying the oral care composition to an inside surface of the mouth of the subject.

In certain non-limiting embodiments, the oral composition described herein comprises: (i) a plant extract selected from the group consisting of vanilla extract, lemon extract, citrus extract, grapefruit seed extract and combinations thereof at a concentration of between about 0.05 and 1% w/w, or between about 0.1 and 0.6% w/w;

(ii) polyhexamethylene biguanide (PHMB), polyaminopropyl biguanide (PAPB) or combinations thereof at a concentration between about 0.001 and 5% w/w, or between about 0.005 and 1% w/w, or between about 0.01 and 0.3% w/w; and

(iii) one or more essential oil or active ingredient thereof selected from the group consisting of lemongrass oil, lemon oil, fennel seed oil, orange oil, basil oil, cinnamon oil, thyme oil, clove oil, pine oil, cedar wood oil, thymol, farnesol, bisabolol, cinnamaldehyde, cinnamyl acetic ester, cinnamic acid, ethyl cinnamate or combinations thereof, at a concentration of between about 0.005 and 1% w/w, or between about 0.01 and 0.5% w/w.

In certain embodiments, the composition comprises vanilla extract, lemon extract or combinations thereof, and does not comprise any other citrus extract.

In certain non-limiting embodiments, the composition described herein further comprises: (iv) one or more alkanediol selected from the group consisting of propanediol, pentanediol, hexanediol, heptanediol, octanediol, decanediol, nonanediol and combinations thereof, at a concentration of between about 0 and 10% w/w, or between about 0 and 5% w/w, or between about 0.01 and 0.5% w/w;

(v) an organic acid selected from the group consisting of benzoic acid, lactic acid, citric acid and combinations thereof, at a concentration of between about 0.01 and 1% w/w, or between about 0.05 and 0.5% w/w;

(vi) a solvents which enhances the solubility of the ingredients of the composition, selected from the group consisting of alcohol (between about 0 and 80% w/w), phenyl ethanol (between about 0 and 1.0% w/w), a mixture of PEG-40 hydrogenated castor oil, Trideceth-9, propylene glycol and water (between about 0 and 2% w/w), a mixture of PEG-40 hydrogenated castor oil, Trideceth-9 and water (between about 0 and 2% w/w), caprylyl capryl glucoside (Plantasol) (between about 0 and 2% w/w), and combinations thereof;

(vii) one or more anti-irritant selected from the group consisting of zinc salicylate, basil oil, brahmi extract, red sandal wood extract, curcumin compound, aloe, bisabolol, rosemary oil, chalmogra oil, oat powder and/or extract, beta glucan, chitosan, calendula oil, portulaca extract, witch hazel extract, pomegranate oil, at a concentration of between about 0.05 and 5% w/w, or between about 0.1 and 2% w/w.

In certain non-limiting embodiments, the composition described herein comprises: (i) a plant extract selected from the group consisting of vanilla extract, lemon extract and combinations thereof at a concentration of between about 0.1 and 0.6% w/w; (ii) polyhexamethylene biguanide (PHMB) at a concentration of between about 0.01 and 0.3% w/w; and (iii) one or more essential oil or active ingredient thereof selected from the group consisting of thyme oil, cinnamon oil, thymol, cinnamaldehyde, cinnamylacetic ester, cinnamic acid, ethyl cinnamate or combinations thereof, at a concentration of between about 0.01 and 0.5% w/w.

In certain non-limiting embodiments, an oral care rinse, gel or toothpaste is provided comprising (a) one or more plant/fruit extract which may be citrus extract (e.g. lemon extract, grapefruit seed extract), vanilla extract, wasabi extract or a combination thereof (e.g. at a concentration of between about 0.1 and 0.6% w/w); (b) one or more synthetic antimicrobial which may be cetyl pyrinidium chloride or a biguanide such as chlorhexidine free base or chlorhexidine salt (e.g. chlorhexidine gluconate or chlorhexidine acetate) or polyhexamethylene biguanide, polyaminopropyl biguanide, or a combination thereof (e.g. at a concentration of between about 0.05 and 0.5% w/w); (c) one or more alkanediol which may be propanediol, pentanediol or a combination thereof (e.g. at a concentration between about 0.5 and 5.0% w/w or between about 0.5 and 2.0% w/w); (d) one or more essential oil or constituent thereof which may be thymol, a structural isomer of thymol (such as, for example, carvacrol or dekasol BL (e.g., o-Cymen-5-ol)), rosemary oil, cinnamon oil, clove bud oil, basil oil, or a combination thereof (e.g. at a concentration between about 0.05-0.5% w/w); (e) one or more flavoring agent which may be spearmint oil, peppermint oil, menthol, or a combination thereof (e.g. at a concentration between about 0.0-0.2% w/w); (f) one or more plant-based mild solubilizing non-ionic surfactant, for example caprilyl capryl glucoside or decyl glucoside (e.g. at a concentration between about 0.3 and 10.0% w/w); (g) one or more plant-based organic acid which may be lactic acid, citric acid, benzoic acid or a combination thereof (e.g. at a concentration between about 0.1 and 0.5% w/w); (h) one or more botanical anti-irritant, for example aloe gel/extract (e.g. at a concentration between about 0.1 and 1.0% w/w); and/or (i) one or more anti-inflammatory agent which may be calendula oil, rosemary oil, a curcumin compound (e.g. a tetrahydrocurcuminoid compound), pomegranate oil, Echinacea purpurea extract, Portulaca extract, resveratrol, zinc salicylate or a combination thereof (e.g. at a concentration between about 0.1 and 0.5% w/w). Said composition may optionally further comprise one or more of (i) an alcohol at a concentration up to about 25% w/w or up to about 20% w/w or up to about 15% w/w or up to about 10% w/w or up to about 5% w/w; (ii) a solubilizer containing mixture of PEG-40 Hydrogenated castor oil, Tridecath 9 and water (e.g. at a concentration between about 0.5 and 5.0% w/w); (iii) one or more emollient which may be propylene glycol, dipropylene glycol, glycerin, diglycerin or a combination thereof (e.g. at a concentration between about 0.5 and 10.0% w/w); (iv) one or more gelling agent which may be hydroxypropylmethyl cellulose, hydroxy propyl methyl cellulose stearoxy ether (Sangelose), chitosan, pyrrolidone carboxylate (Kytamer), Poloxamer (pluronic gel) or a combination thereof (e.g. at a concentration between about 0.01 and 1.0% w/w); and/or (v) a sweetener, for example a sucralose-based sweetener such as Splenda, xylitol, and/or sorbitol.

In certain non-limiting embodiments, a botanical oral rinse is provided comprising the following ranges of ingredients:

| Ingredients | % w/w |
|---|---|
| Water | 50.0-90.0 |
| Kytomer | 0.05-0.5 |
| Zinc salicylate | 0.01-1.0 |
| Pluronic F-87/F-127 | 0.05-2.0 |
| Lactic acid | 0.05-2.0 |
| Splenda sugar | 0.05-5.0 |
| Citrus extract | 0.01-2.0 |
| Wasabi | 0-3.0 |
| PHMB | 0.05-1.0 |
| Benzoic acid | 0.1-1.0 |
| Clove bud oil | 0.01-1.0 |
| Thymol | 0.01-0.5 |
| Glycerine | 3-15 |
| Menthol | 0.01-1.0 |
| Spearmint oil | 0.01-2.0 |
| Delmopinol hydrochloride | 0-0.5 |
| or CetylPyridinium chloride | |
| Peppermint oil | 0.01-2.0 |
| Rosemary oil | 0.01-2.0 |
| Plantasol | 0.5-5.0 |
| Cinnamon bark oil | 0.01-2.0 |
| Solubulizer (Peg-40 | 0.0-3.0 |
| hydrogented Castor | |
| oil, Trideceth 9, water) | |
| Pentylene glycol | 0.5-2.0 |
| Adjust pH to 3.5-3.9 | |

In a specific non-limiting embodiment, a botanical oral rinse is provided comprising the following concentrations within the ranges set forth in the preceding paragraph:

| Ingredients | % w/w |
|---|---|
| Water | 85.75 |
| Kytomer | 0.2 |
| Zinc salicylate | 0.05 |
| Pluronic F-87 | 0.2 |
| Lactic acid | 0.2 |
| Splenda sugar | 0.2 |
| Citrus extract | 0.5 |
| Wasabi | 0.3 |
| PHMB | 0.1 |
| Benzoic acid | 0.2 |
| Clove bud oil | 0.03 |
| Thymol | 0.05 |
| Glycerine | 7.0 |
| Menthol | 0.05 |
| Spearmint oil | 0.02 |
| Peppermint oil | 0.02 |
| Rosemary oil | 0.1 |
| Plantasol | 3.0 |
| Cinnamon bark oil | 0.05 |
| Solubulizer (Peg-40 | 1.0 |
| hydrogented Castor oil, | |
| Trideceth 9, water) | |
| Pentylene glycol | 1.0 |
| Adjust pH to 3.5-3.9 | |

In certain non-limiting embodiments, a botanical oral rinse is provided comprising the following ranges of ingredients:

| Ingredients | % w/w |
|---|---|
| Lemon extract | 0.1-0.6 |
| PHMB | 0.06-0.12 |
| Cinnamon bark oil | 0.02-0.2 |
| Basil | 0.0-0.2 |
| Rosemary | 0.0-0.2 |
| Lemon grass oil | 0.00-0.2 |
| Thymol | 0.02-0.1 |
| Menthol | 0.02-0.1 |
| Clove bud oil | 0.01-0.1 |
| Peppermint | 0.02-0.2 |
| Xylitol | 0.25-1 |
| Sorbitol | 0.25-5.0 |
| Poloxamer | 0.05-1.0 |
| (Pluronic F-87/F127) | |
| Zinc salicylate | 0.05-0.2 |
| Lactic acid | 0.02-0.2 |
| Glycerin | 2-15 |
| Propylene glycol | 0-8 |
| Zemea | 1-7 |
| Aloe leaf juice | 0.05-2 |
| Plantasol (Caprylyl | 0.0-2.0 |
| capryl Glucoside) | |
| Water | add to make 100% |

In a specific non-limiting embodiment, a botanical oral rinse is provided comprising the following concentrations within the ranges set forth in the preceding paragraph:

| Ingredients | % w/w |
|---|---|
| Lemon extract | 0.3 |
| PHMB | 0.06 |
| Cinnamon bark oil | 0.05 |
| Basil | 0.02 |
| Rosemary | 0.03 |
| Lemon grass oil | 0.02 |
| Thymol | 0.05 |
| Menthol | 0.02 |
| Clove bud oil | 0.02 |
| Peppermint | 0.02 |
| Water | 80.36 |
| Xylitol | 0.75 |
| Sorbitol | 1.0 |
| Pluronic F 087 | 0.1 |
| Zinc salicylate | 0.1 |
| Lactic acid | 0.1 |
| Glycerin | 7.0 |
| Propylene glycol | 6.0 |
| Zemea | 2.0 |
| Aloe leaf juice | 1.0 |
| Plantasol (Caprylyl | 1.0 |
| capryl Glucoside) | |

In certain non-limiting embodiments, a botanical oral rinse is provided comprising the following ranges of ingredients:

| Ingredients | % w/w |
|---|---|
| Lemon extract | 0.1-0.6 |
| Vanilla extract | 0.0-0.6 |
| PHMB | 0.06-0.12 |
| Cinnamon leaf/bark oil | 0.02-0.2 |
| Basil | 0.00-0.2 |
| Rosemary | 0.0-0.2 |
| Lemon grass oil | 0.00-0.2 |
| Thymol | 0.02-0.1 |
| Menthol | 0.02-0.2 |
| Clove bud oil | 0.01-0.1 |
| Peppermint | 0.02-0.2 |
| Alcohol | 0-15 |
| Water | 65-78 |
| Xylitol | 0.25-1 |
| Sorbitol | 0.25-7.0 |
| Poloxamer | 0.05-1.0 |
| (Pluronic F127 or F87) | |
| Zinc salicylate | 0.05-0.2 |
| Lactic acid | 0.02-0.2 |
| Glycerin | 2-15 |
| Propylene glycol | 0-10 |
| Zemea | 1-7 |
| Aloe leaf juice | 0.05-2 |
| Plantasol (Caprylyl | 0.0-2.0 |
| capryl Glucoside) | |
| Pentylene Glycol | 0-1.0 |
| Tetra Hydro | 0.0-0.1 |
| curcuminoid | |
| Calendula Extract | 0-0.5 |
| Portulaca Extract | 0-0.5 |

In a specific non-limiting embodiment, a botanical oral rinse is provided comprising the following concentrations within the ranges set forth in the preceding paragraph

| Ingredients | % w/w |
|---|---|
| Lemon extract | 0.3 |
| PHMB | 0.06 |
| Cinnamon bark oil | 0.05 |
| Basil | 0.02 |
| Rosemary | 0.03 |
| Lemon grass oil | 0.02 |
| Thymol | 0.05 |
| Menthol | 0.02 |
| Clove bud oil | 0.02 |
| Peppermint | 0.02 |
| Alcohol | 5.0 |
| Water | 75.36 |
| Xylitol | 0.75 |
| Sorbitol | 1.0 |
| Pluronic F 87 | 0.1 |
| Zinc salicylate | 0.1 |
| Lactic acid | 0.1 |
| Glycerin | 7.0 |
| Propylene glycol | 6.0 |
| Zemea | 2.0 |
| Aloe leaf juice | 1.0 |
| Plantasol (Caprylyl | 1.0 |
| capryl Glucoside) | |

In a specific non-limiting embodiment, a botanical oral rinse is provided comprising the following concentrations within the ranges set forth in the preceding paragraph:

| Ingredients | % w/w |
|---|---|
| Tetrahydrocurcumin | 0.02-0.1 |
| Lemon extract | 0.1-0.6 |
| PHMB | 0.03-0.12 |
| Cinnamon bark oil | 0.02-0.2 |
| Basil | 0.02-0.2 |
| Rosemary | 0.01-0.2 |
| Lemon grass oil | 0.01-0.2 |
| Thymol | 0.02-0.6 |
| Menthol | 0.02-0.4 |
| Clove bud oil | 0.01-0.1 |
| Peppermint | 0.02-0.4 |
| Alcohol | 5-15 |
| Water | 65-80 |
| Xylitol | 0.25-1 |
| Sorbitol | 0.25-1.5 |
| Pluronic F 087 | 0.05-0.3 |
| Zinc salicylate | 0.05-0.2 |
| Lactic acid | 0.02-0.2 |
| Glycerin | 2-8 |
| Propylene glycol | 2-8 |
| Zemea | 1-5 |
| Aloe leaf juice | 0.05-2 |
| Pentylene glycol | 0.5-1.0 |
| Plantasol (Caprylyl | 0.5-2.0 |
| capryl Glucoside) | |

In a specific non-limiting embodiment, a botanical oral rinse is provided comprising the following concentrations within the ranges set forth in the preceding paragraph:

| Ingredients | % w/w |
|---|---|
| Tetrahydrocurcumin | 0.05 |
| Lemon extract | 0.3 |
| PHMB | 0.06 |
| Cinnamon bark oil | 0.05 |
| Basil | 0.02 |
| Rosemary | 0.05 |
| Lemon grass oil | 0.02 |
| Thymol | 0.05 |
| Menthol | 0.02 |
| Clove bud oil | 0.02 |
| Peppermint | 0.02 |
| Alcohol | 10.0 |
| Water | 69.79 |
| Xylitol | 0.75 |
| Sorbitol | 1.0 |
| Pluronic F 087 | 0.1 |
| Zinc salicylate | 0.1 |
| Lactic acid | 0.1 |
| Glycerin | 7.0 |
| Propylene glycol | 6.0 |
| Zemea | 2.0 |
| Aloe leaf juice | 1.0 |
| Pentylene glycol | 0.5 |
| Plantasol (Caprylyl | 1.0 |
| capryl Glucoside) | |

In certain non-limiting embodiments, a botanical oral rinse is provided comprising the following ranges of ingredients:

| Ingredients | % w/w |
|---|---|
| Calendula Extract | 0.1-0.5 |
| Lemon extract | 0.1-0.6 |
| PHMB | 0.03-0.12 |
| Cinnamon bark oil | 0.02-0.2 |
| Basil | 0.02-0.2 |
| Rosemary | 0.01-0.2 |
| Lemon grass oil | 0.01-0.2 |
| Thymol | 0.02-0.6 |
| Menthol | 0.02-0.4 |
| Clove bud oil | 0.01-0.1 |
| Peppermint | 0.02-0.4 |
| Alcohol | 5-15 |
| Water | 65-75 |
| Xylitol | 0.25-1 |
| Sorbitol | 0.25-1.5 |
| Pluronic F 087 | 0.05-0.3 |
| Zinc salicylate | 0.05-0.2 |
| Lactic acid | 0.02-0.2 |
| Glycerin | 2-8 |
| Propylene glycol | 2-8 |
| Zemea | 1-5 |
| Aloe leaf juice | 0.05-2 |
| Pentylene glycol | 0.5-1.0 |
| Plantasol (Caprylyl | 0.5-2.0 |
| capryl Glucoside) | |

In a specific non-limiting embodiment, a botanical oral rinse is provided comprising the following concentrations within the ranges set forth in the preceding paragraph:

| Ingredients | % w/w |
|---|---|
| Calendula Extract | 0.1 |
| Lemon extract | 0.3 |
| PHMB | 0.06 |
| Cinnamon bark oil | 0.05 |
| Basil | 0.02 |
| Rosemary | 0.05 |
| Lemon grass oil | 0.02 |
| Thymol | 0.05 |
| Menthol | 0.02 |
| Clove bud oil | 0.02 |
| Peppermint | 0.02 |
| Alcohol | 10.0 |
| Water | 69.67 |
| Xylitol | 0.75 |
| Sorbitol | 1.0 |
| Pluronic F 087 | 0.1 |
| Zinc salicylate | 0.1 |
| Lactic acid | 0.1 |
| Glycerin | 7.0 |
| Propylene glycol | 6.0 |
| Zemea | 2.0 |
| Aloe leaf juice | 1.0 |
| Pentylene glycol | 0.5 |
| Plantasol (Caprylyl | 1.0 |
| capryl Glucoside) | |

In certain non-limiting embodiments, a botanical oral gel is provided comprising the following ranges of ingredients:

| Ingredients | % w/w |
|---|---|
| Water | 50.0-90.0 |
| Kytomer | 0.05-0.5 |
| Hydroxypropyl methyl- | 0.05-0.5 |
| cellulose stearoxy | |
| ether (Sangelose) | |
| Zinc salicylate | 0.01-1.0 |
| Citrus extract | 0.01-2.0 |
| Wasabi | 0-3.0 |
| PHMB | 0.05-1.0 |
| Clove bud oil | 0.01-1.0 |
| Thymol | 0.01-0.5 |
| Menthol | 0.01-1.0 |
| Calendula oil | 0.05-2.0 |
| Tetrahydrocurcumanoid | 0.01-1.0 |
| Pomegranate oil | 0.05-2.0 |
| Spearmint oil | 0.05-2.0 |
| Peppermint oil | 0.05-2.0 |
| Cinnamon bark oil | 0.01-1.0 |
| Pentylene glycol | 0.5-2.0 |
| Natural Glycerine | 5-20 |
| Adjust pH to 3.5-3.9 | |

In a specific non-limiting embodiment, a botanical oral gel is provided comprising the following concentrations within the ranges set forth in the preceding paragraph:

| Ingredients | % w/w |
|---|---|
| Water | 86.88 |
| Kytomer | 0.2 |
| Hydroxypropyl methyl- | 0.3 |
| cellulose stearoxy ether (Sangelose) | |
| Zinc salicylate | 0.05 |
| Citrus extract | 0.5 |
| Wasabi | 0.3 |
| PHMB | 0.1 |
| Clove bud oil | 0.03 |
| Thymol | 0.05 |
| Menthol | 0.05 |
| Calendula extract | 0.2 |
| Tetrahydrocurcumanoid | 0.05 |
| Pomegranate oil | 0.2 |
| Spearmint oil | 0.02 |
| Peppermint oil | 0.02 |
| Cinnamon bark oil | 0.05 |
| Pentylene glycol | 1.0 |
| Natural Glycerine | 10.0 |
| Adjust pH to 3.5-3.9 | |

In a specific non-limiting embodiment, a botanical oral rinse is provided (referred to as “OCP-G1”) comprising the following concentrations, where phases A and B may be prepared separately then mixed:

| Ingredients | (% w/w) |
|---|---|
| Phase A | |
| Water | 83.09 |
| Xylitol | 0.25 |
| Sorbitol | 0.5 |
| Pluronic F127 | 0.5 |
| Lactic acid | 0.1 |
| Zinc salicylate | 0.1 |
| Glycerin | 7.0 |
| Propylene glycol | 1.0 |
| Zemea | 5.0 |
| Aloe leaf juice | 1.0 |
| Phase B | |
| Vanilla extract | 0.6 |
| PHMB | 0.12 |
| Cinnamon bark oil | 0.05 |
| Lemon grass oil | 0.01 |
| Clove bud oil | 0.03 |
| Peppermint | 0.05 |
| Hydrolite 5 | 0.5 |
| Polysorbate 80 | 0.1 |

In a specific non-limiting embodiment, a botanical oral rinse is provided (referred to as “OCP-G1”) comprising the following concentrations, where phases A and B may be prepared separately then mixed:

| Ingredients | (% w/w) |
|---|---|
| Phase A | |
| Water | 85.59 |
| Xylitol | 0.25 |
| Sorbitol | 0.5 |
| Pluronic F087 | 0.1 |
| Lactic acid | 0.1 |
| Zinc salicylate | 0.1 |
| Glycerin | 7.0 |
| Propylene glycol | 3.0 |
| Aloe leaf juice | 1.0 |
| Phase B | |
| Vanilla extract | 0.6 |
| PHMB | 0.12 |
| Cinnamon bark oil | 0.05 |
| Lemon grass oil | 0.01 |
| Clove bud oil | 0.03 |
| Peppermint | 0.05 |
| Hydrolite 5 | 0.5 |
| Polysorbate 80 | 1.0 |

In a specific non-limiting embodiment, a botanical oral rinse is provided (referred to as “OCP-G2”) comprising the following concentrations, where phases A and B may be prepared separately and then mixed:

| Ingredients | (% w/w) |
|---|---|
| Phase A | |
| Water | 86.49 |
| Xylitol | 0.25 |
| Sorbitol | 0.5 |
| Pluronic F087 | 0.1 |
| Lactic acid | 0.1 |
| Zinc salicylate | 0.1 |
| Glycerin | 7.0 |
| Propylene glycol | 3.0 |
| Aloe leaf juice | 1.0 |
| Phase B | |
| Lemon extract | 0.6 |
| PHMB | 0.12 |
| Cinnamon bark oil | 0.05 |
| Lemon grass oil | 0.01 |
| Clove bud oil | 0.03 |
| Peppermint | 0.05 |
| Hydrolite 5 | 0.5 |
| Polysorbate 80 | 0.1 |

In a specific non-limiting embodiment, a botanical oral rinse is provided (referred to as “OCP-G2*”) comprising the following concentrations, where phases A and B may be prepared separately then mixed:

| Ingredients | (% w/w) |
|---|---|
| Phase A | |
| Water | 85.59 |
| Xylitol | 0.25 |
| Sorbitol | 0.5 |
| Pluronic F087 | 0.1 |
| Lactic acid | 0.1 |
| Zinc salicylate | 0.1 |
| Glycerin | 7.0 |
| Propylene glycol | 3.0 |
| Aloe leaf juice | 1.0 |
| Phase B | |
| Lemon extract | 0.6 |
| PHMB | 0.12 |
| Cinnamon bark oil | 0.05 |
| Lemon grass oil | 0.01 |
| Clove bud oil | 0.03 |
| Peppermint | 0.05 |
| Hydrolite 5 | 0.5 |
| Polysorbate 80 | 1.0 |

In a specific non-limiting embodiment, a botanical oral rinse with anti-inflammatory activity is provided (referred to as “OCP-AIF”) comprising the following concentrations, where phases A and B may be prepared separately and then mixed:

| Ingredients | (% w/w) |
|---|---|
| Phase A | |
| Water | 73.44 |
| Xylitol | 0.25 |
| Sorbitol | 0.5 |
| Pluronic F087 | 0.1 |
| Lactic acid | 0.1 |
| Zinc salicylate | 0.1 |
| Glycerin | 10.0 |
| Propylene glycol | 3.0 |
| Aloe leaf juice | 1.0 |
| Phase B | |
| Alcohol | 10 |
| Tetrahydro curcumin | 0.05 |
| Rosemary | 0.1 |
| PHMB | 0.12 |
| Cinnamon bark oil | 0.05 |
| Lemon grass oil | 0.01 |
| Clove bud oil | 0.03 |
| Peppermint | 0.05 |
| Hydrolite 5 | 0.5 |
| Solubilizer | 0.5 |
| Polysorbate 80 | 0.1 |

In a specific non-limiting embodiment, a botanical oral rinse with anti-inflammatory activity is provided (referred to as “OCP-AIF*”) comprising the following concentrations, where phases A and B may be prepared separately and then mixed:

| Ingredients | (% w/w) |
|---|---|
| Phase A | |
| Water | 75.54 |
| Xylitol | 0.25 |
| Sorbitol | 0.5 |
| Pluronic F087 | 0.1 |
| Lactic acid | 0.1 |
| Zinc salicylate | 0.1 |
| Glycerin | 7.0 |
| Propylene glycol | 3.0 |
| Aloe leaf juice | 1.0 |
| Phase B | |
| Alcohol | 10 |
| Tetrahydro curcumin | 0.05 |
| Rosemary | 0.1 |
| PHMB | 0.12 |
| Cinnamon bark oil | 0.05 |
| Lemon grass oil | 0.01 |
| Clove bud oil | 0.03 |
| Peppermint | 0.05 |
| Hydrolite 5 | 0.5 |
| Solubilizer | 0.5 |
| Polysorbate 80 | 1.0 |

In a specific non-limiting embodiment, a botanical oral rinse (referred to as “OCP-M20”) is provided comprising the following ranges of ingredients:

| Ingredients | % w/w |
|---|---|
| Lemon extract | 0.1-0.6 |
| PHMB | 0.06-0.12 |
| Cinnamon bark oil | 0.02-0.2 |
| Thymol | 0.02-0.6 |
| Clove bud oil | 0.01-0.1 |
| Peppermint | 0.02-0.2 |
| Water | 50-98 |
| Xylitol | 0.25-1 |
| Sorbitol | 1-7 |
| Pluronic F 127 | 0.5-1.5 |
| Zinc salicylate | 0.05-0.2 |
| Lactic acid | 0.02-0.2 |
| Glycerin | 5-15 |
| Zemea | 1-5 |
| Aloe leaf juice | 0.05-2 |
| Pentylene glycol | 0.2-1.0 |
| Plantasol (Caprylyl capryl Glucoside) | 0.5-1.0 |

In a specific non-limiting embodiment, a botanical oral rinse (referred to as OCP-M20-1) is provided comprising the following concentrations within the ranges set forth in the preceding paragraph:

| Ingredients | % w/w |
|---|---|
| Lemon extract | 0.3 |
| PHMB | 0.06 |
| Cinnamon bark oil | 0.05 |
| Thymol | 0.05 |
| Clove bud oil | 0.02 |
| Peppermint | 0.03 |
| Water | 73.79 |
| Xylitol | 0.5 |
| Sorbitol | 5.0 |
| Pluronic F 127 | 1.0 |
| Zinc salicylate | 0.1 |
| Lactic acid | 0.1 |
| Glycerin | 12.0 |
| Zemea | 5.0 |
| Aloe leaf juice | 1.0 |
| Pentylene glycol | 0.5 |
| Plantasol (Caprylyl capryl Glucoside) | 0.5 |

In a specific non-limiting embodiment, a botanical oral rinse (referred to as “OCP-M21”) is provided comprising the following ranges of ingredients:

| Ingredients | % w/w |
|---|---|
| Lemon extract | 0.1-0.6 |
| Vanilla extract | 0.1-0.6 |
| Calendula extract | 0.2-1.0 |
| PHMB | 0.06-0.12 |
| Cinnamon bark oil | 0.02-0.2 |
| Thymol | 0.02-0.6 |
| Clove bud oil | 0.01-0.1 |
| Peppermint | 0.02-0.2 |
| Water | 50-98 |
| Xylitol | 0.25-1 |
| Sorbitol | 0.25-7 |
| Pluronic F 127 | 0.5-1.5 |
| Zinc salicylate | 0.05-0.2 |
| Lactic acid | 0.02-0.2 |
| Glycerin | 5-15 |
| Zemea | 1-5 |
| Aloe leaf juice | 0.05-2 |
| Pentylene glycol | 0.2-1.0 |
| Plantasol (Caprylyl capryl Glucoside) | 0.5-2.0 |

In a specific non-limiting embodiment, a botanical oral rinse (referred to as OCP-M21-1) is provided comprising the following concentrations within the ranges set forth in the preceding paragraph:

| Ingredients | % w/w |
|---|---|
| Lemon extract | 0.15 |
| Vanilla extract | 0.15 |
| Calendula extract | 0.5 |
| PHMB | 0.06 |
| Cinnamon bark oil | 0.05 |
| Thymol | 0.05 |
| Clove bud oil | 0.02 |
| Peppermint | 0.03 |
| Water | 73.29 |
| Xylitol | 0.5 |
| Sorbitol | 5.0 |
| Pluronic F 127 | 1.0 |
| Zinc salicylate | 0.1 |
| Lactic acid | 0.1 |
| Glycerin | 12.0 |
| Zemea | 5.0 |
| Aloe leaf juice | 1.0 |
| Pentylene glycol | 0.5 |
| Plantasol (Caprylyl capryl Glucoside) | 0.5 |

In a specific non-limiting embodiment, a botanical oral rinse (referred to as “OCP-AIFCT4”) is provided comprising the following ranges of ingredients:

| Ingredients | % w/w |
|---|---|
| Lemon extract | 0.1-0.6 |
| Calendula extract | 0.2-1.0 |
| PHMB | 0.06-0.12 |
| Cinnamon bark oil | 0.02-0.2 |
| Thymol | 0.02-0.6 |
| Clove bud oil | 0.01-0.1 |
| Peppermint | 0.02-0.2 |
| Alcohol | 5-12 |
| Tetrahydrocurcumin | 0.02-0.1 |
| Water | 50-98 |
| Xylitol | 0.25-1 |
| Sorbitol | 1-7 |
| Pluronic F 127 | 0.5-1.5 |
| Zinc salicylate | 0.05-0.2 |
| Lactic acid | 0.02-0.2 |
| Glycerin | 5-15 |
| Zemea | 1-5 |
| Aloe leaf juice | 0.05-2 |
| Pentylene glycol | 0.2-1.0 |
| Plantasol (Caprylyl capryl Glucoside) | 0.5-2.0 |

In a specific non-limiting embodiment, a botanical oral rinse (referred to as “OCP-AIFCT4-1”) is provided comprising the following concentrations within the ranges set forth in the preceding paragraph:

| Ingredients | % w/w |
|---|---|
| Lemon extract | 0.3 |
| Calendula extract | 0.5 |
| PHMB | 0.12 |
| Cinnamon bark oil | 0.05 |
| Thymol | 0.05 |
| Clove bud oil | 0.02 |
| Peppermint | 0.03 |
| Alcohol | 10 |
| Tetrahydrocurcumin | 0.05 |
| Water | 63.18 |
| Xylitol | 0.5 |
| Sorbitol | 5.0 |
| Pluronic F 127 | 1.0 |
| Zinc salicylate | 0.1 |
| Lactic acid | 0.1 |
| Glycerin | 12.0 |
| Zemea | 5.0 |
| Aloe leaf juice | 1.0 |
| Pentylene glycol | 0.5 |
| Plantasol (Caprylyl capryl Glucoside) | 0.5 |

In a specific non-limiting embodiment, an oral rinse (referred to as “Oral rinse OCP-1”) is provided comprising the following:

| Ingredients | % w/w |
|---|---|
| Phase A (Other ingredients) | |
| Water | 83.09 |
| Xylitol | 0.25 |
| Sorbitol | 0.5 |
| Pluronic F127 | 0.5 |
| Lactic acid | 0.1 |
| Zinc salicylate | 0.1 |
| Glycerin | 7.0 |
| Propylene glycol | 1.0 |
| Zemea | 5.0 |
| Aloe leaf juice | 1.0 |
| Phase B (Main ingredients) | |
| Vanilla extract | 0.6 |
| PHMB | 0.12 |
| Cinnamon bark oil | 0.05 |
| Lemon grass oil | 0.01 |
| Clove bud oil | 0.03 |
| Peppermint | 0.05 |
| Hydrolite 5 | 0.5 |
| Polysorbate 80 | 0.1 |

In a specific non-limiting embodiment, an oral rinse (referred to as “Oral rinse OCP-M”) is provided comprising the following:

| Ingredients | % w/w |
|---|---|
| Lemon extract | 0.6 |
| Cinnamon bark oil | 0.05 |
| Thymol | 0.05 |
| Clove bud oil | 0.03 |
| Basil | 0.05 |
| Menthol | 0.05 |
| Spearmint oil | 0.04 |
| Eucalyptus | 0.03 |
| Lemon grass oil | 0.05 |
| Rosemary oil | 0.05 |
| PHMB | 0.06 |
| Peppermint oil | 0.03 |
| Water | 73.29 |
| Xylitol | 0.25 |
| Sorbitol | 0.75 |
| Pluronic F 087 | 0.1 |
| Zinc salicylate | 0.1 |
| Lactic acid | 0.1 |
| Glycerin | 7.0 |
| Zemea | 2.0 |
| Aloe leaf juice | 1.0 |
| Propylene glycol | 6.0 |
| Plantasol (Caprylyl capryl Glucoside) | 1.0 |

In a specific non-limiting embodiment, an oral rinse (referred to as “6B”) is provided comprising the following:

| Main ingredients | % w/w |
|---|---|
| Vanilla extract | 0.6 |
| Thymol | 0.05 |
| Cinnamon bark oil | 0.05 |
| PHMB | 0.1 |
| Benzoic acid | 0.1 |
| Other ingredients | |
| Water | 76.45 |
| Pluronic F-127 | 1.0 |
| Sorbitol | 5.0 |
| Xylitol | 0.5 |
| Zinc salicylate | 0.1 |
| Glycerin | 12.0 |
| Zemea (1,2-propanediol) | 4.0 |
| Peppermint oil | 0.05 |

In a specific non-limiting embodiment, an oral rinse (referred to as “7B”) is provided comprising the following:

| Main ingredients | % w/w |
|---|---|
| Vanilla extract | 0.6 |
| Thymol | 0.05 |
| Cinnamon bark oil | 0.05 |
| PHMB | 0.06 |
| Benzoic acid | 0.1 |
| Other ingredients | |
| Water | 76.45 |
| Pluronic F-127 | 1.0 |
| Sorbitol | 5.0 |
| Xylitol | 0.5 |
| Zinc salicylate | 0.1 |
| Glycerin | 12.0 |
| Zemea (1,2-propanediol) | 4.0 |
| Peppermint oil | 0.05 |

In a specific non-limiting embodiment, an oral rinse (referred to as “8B”) is provided comprising the following:

| Main ingredients | % w/w |
|---|---|
| Vanilla extract | 0.6 |
| Thymol | 0.05 |
| Cinnamon bark oil | 0.05 |
| PHMB | 0.03 |
| Benzoic acid | 0.1 |
| Other ingredients | |
| Water | 76.45 |
| Pluronic F-127 | 1.0 |
| Sorbitol | 5.0 |
| Xylitol | 0.5 |
| Zinc salicylate | 0.1 |
| Glycerin | 12.0 |
| Zemea (1,2-propanediol) | 4.0 |
| Peppermint oil | 0.05 |

In a specific non-limiting embodiment, an oral rinse (referred to as “15BCF”) is provided comprising the following:

| Main ingredients | % w/w |
|---|---|
| Vanilla extract | 0.3 |
| Thymol | 0.05 |
| Cinnamon bark oil | 0.01 |
| PHMB | 0.1 |
| Benzoic acid | 0.1 |
| Basil oil | 0.01 |
| Fennel seed oil | 0.05 |
| Other ingredients | |
| Water | 74.73 |
| Pluronic F-127 | 1.0 |
| Sorbitol | 5.0 |
| Xylitol | 0.5 |
| Zinc salicylate | 0.1 |
| Glycerin | 12.0 |
| Zemea (1,2-propanediol) | 4.0 |
| Propylene glycol | 2.0 |
| Peppermint oil | 0.05 |

In a specific non-limiting embodiment, an oral rinse (referred to as “A2”) is provided comprising the following:

| Main ingredients | % w/w |
|---|---|
| Vanilla extract | 0.3 |
| Thymol | 0.05 |
| Cinnamon bark oil | 0.01 |
| PHMB | 0.02 |
| Benzoic acid | 0.1 |
| Basil oil | 0.01 |
| Fennel seed oil | 0.05 |
| Tetrahydrocurcumin | 0.05 |
| Other ingredients | |
| Water | 64.76 |
| Pluronic F-127 | 1.0 |
| Sorbitol | 5.0 |
| Xylitol | 0.5 |
| Zinc salicylate | 0.1 |
| Glycerin | 12.0 |
| Alcohol | 10.0 |
| Zemea (1, 2-propanediol) | 4.0 |
| Propylene glycol | 2.0 |
| Peppermint oil | 0.05 |

In a specific non-limiting embodiment, an oral rinse (referred to as “16BCF”) is provided comprising the following:

| Main ingredients | % w/w |
|---|---|
| Tetrahydrocurcumin | 0.05 |
| Cinnamon bark oil | 0.01 |
| Basil oil | 0.01 |
| Fennel seed oil | 0.05 |
| Thymol | 0.05 |
| Vanilla extract | 0.3 |
| PHMB | 0.1 |
| Other ingredients | |
| Water | 64.28 |
| Zemea | 4.0 |
| Alcohol | 10.0 |
| Pluronic F 127 | 1.0 |
| Benzoic acid | 0.1 |
| Glycerin | 12.0 |
| Zinc salicylate | 0.1 |
| Sorbitol | 5.0 |
| Xylitol | 0.5 |
| Propylene glycol | 2.0 |
| Peppermint oil | 0.05 |

In a specific non-limiting embodiment, an oral rinse (referred to as “BOR-1”) is provided comprising the following:

| Ingredients | % w/w |
|---|---|
| Lemon Extract | 0.6 |
| Synthetic AB X | 0.12 |
| Cinnamon bark oil | 0.07 |
| Lemon grass oil | 0.07 |
| Basil oil | 0.07 |
| Clove bud oil | 0.03 |
| Peppermint | 0.04 |
| Hydrolite 5 | 0.5 |
| Solubilizer (Caprylyl capryl glucoside) | 2.0 |
| Solubilizer (Polyethylene glycol-40 | 0.5 |
| Hydrogenated castor oil) | |
| Water | 80.20 |
| Xylitol | 0.25 |
| Sorbitol | 0.25 |
| Pluronic F87 | 0.2 |
| Citric acid | 0.2 |
| Zinc salicylate | 0.1 |
| Propylene glycol | 5.0 |
| Glycerine | 5.0 |
| Aloe leaf juice | 1.0 |

In a specific non-limiting embodiment, an oral rinse (referred to as “Oral rinse 17BCF.1”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.05 |
| Vanilla extract | 0.3 |
| PHMB | 0.1 |
| Other ingredients | |
| Water | 74.18 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 1.0 |
| Peppermint oil | 0.05 |
| Caprylyl capryl Glucoside (Plantasol) | 0.5 |

In a specific non-limiting embodiment, an oral rinse (referred to as “Oral rinse 17BCF.2”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.1 |
| Vanilla extract | 0.3 |
| PAPB | 0.1 |
| Other ingredients | |
| Water | 74.18 |
| Zem ea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 4.0 |
| Xylitol | 2.0 |
| Peppermint oil | 0.05 |
| Caprylyl capryl Glucoside (Plantasol) | 0.5 |
| Solubilizer (611674) | 0.25 |

In a specific non-limiting embodiment, an oral rinse (referred to as “Oral rinse 17BCF-1”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.075 |
| Vanilla extract | 0.3 |
| PHMB | 0.1 |
| Other ingredients | |
| Water | 74.18 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 1.0 |
| Peppermint oil | 0.05 |
| Caprylyl capryl Glucoside (Plantasol) | 0.5 |

In a specific non-limiting embodiment, an oral rinse (referred to as “Oral rinse 17BCF-2”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.05 |
| Vanilla extract | 0.3 |
| PAPB | 0.10 |
| Other ingredients | |
| Water | 73.68 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 3.0 |
| Xylitol | 2.0 |
| Peppermint oil | 0.05 |
| Caprylyl capryl Glucoside (Plantasol) | 0.5 |

In a specific non-limiting embodiment, an oral rinse (referred to as “Oral rinse 17BCF-2.2”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.1 |
| Vanilla extract | 0.3 |
| PHMB | 0.1 |
| Other ingredients | |
| Water | 74.18 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 4.0 |
| Xylitol | 2.0 |
| Peppermint oil | 0.05 |
| Caprylyl capryl Glucoside (Plantasol) | 0.5 |

In a specific non-limiting embodiment, an oral rinse (referred to as “Oral rinse 17BCF-2A”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.1 |
| Vanilla extract | 0.3 |
| PAPB | 0.10 |
| Other ingredients | |
| Water | 73.68 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 4.0 |
| Xylitol | 2.0 |
| Peppermint oil | 0.05 |
| Caprylyl capryl Glucoside (Plantasol) | 0.5 |

In a specific non-limiting embodiment, an oral rinse (referred to as “Oral rinse 17BCF-1A”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.075 |
| Vanilla extract | 0.3 |
| PAPB | 0.1 |
| Other ingredients | |
| Water | 74.18 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 1.0 |
| Peppermint oil | 0.05 |
| Caprylyl capryl Glucoside (Plantasol) | 0.5 |

In a specific non-limiting embodiment, an oral rinse (referred to as “Oral rinse 18BCF.1”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.05 |
| Vanilla extract | 0.3 |
| PHMB | 0.12 |
| Other ingredients | |
| Water | 74.16 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |
| Caprylyl capryl Glucoside (Plantasol) | 0.5 |

In a specific non-limiting embodiment, an oral rinse (referred to as “Oral rinse 18BCF.2”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.05 |
| Vanilla extract | 0.3 |
| PAPB | 0.12 |
| Other ingredients | |
| Water | 74.16 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |
| Caprylyl capryl Glucoside (Plantasol) | 0.5 |

In a specific non-limiting embodiment, an oral rinse (referred to as “Oral rinse 19BCF-1”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.075 |
| Vanilla extract | 0.3 |
| PAPB | 0.08 |
| Other ingredients | |
| Water | 74.2 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 1.0 |
| Peppermint oil | 0.05 |
| Caprylyl capryl Glucoside (Plantasol) | 0.5 |

In a specific non-limiting embodiment, an oral rinse (referred to as “Oral rinse 19BCF-1A”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.05 |
| Vanilla extract | 0.3 |
| PAPB | 0.08 |
| Other ingredients | |
| Water | 73.68 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 1.0 |
| Peppermint oil | 0.05 |
| Caprylyl capryl Glucoside (Plantasol) | 0.5 |

In a specific non-limiting embodiment, an oral rinse (referred to as “Oral rinse 19BCF-2A”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.05 |
| Vanilla extract | 0.3 |
| PAPB | 0.08 |
| Other ingredients | |
| Water | 73.7 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 4.0 |
| Xylitol | 2.0 |
| Peppermint oil | 0.05 |
| Caprylyl capryl Glucoside (Plantasol) | 0.5 |

In a specific non-limiting embodiment, an oral rinse (referred to as “Oral rinse 19BCF.2”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.1 |
| Vanilla extract | 0.3 |
| PAPB | 0.08 |
| Other ingredients | |
| Water | 74.2 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 4.0 |
| Xylitol | 2.0 |
| Peppermint oil | 0.05 |
| Caprylyl capryl Glucoside (Plantasol) | 0.5 |

In a specific non-limiting embodiment, an oral rinse (referred to as “Oral rinse 19BCF-2”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.05 |
| Vanilla extract | 0.3 |
| PAPB | 0.08 |
| Other ingredients | |
| Water | 73.7 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 3.0 |
| Xylitol | 2.0 |
| Peppermint oil | 0.05 |
| Caprylyl capryl Glucoside (Plantasol) | 0.5 |

In a specific non-limiting embodiment, an oral rinse (referred to as “Oral rinse A3.1”) is provided comprising the following:

| Main ingredients | % w/w |
|---|---|
| Tetrahydrocurcumin | 0.05 |
| Cinnamon bark oil | 0.01 |
| Basil oil | 0.01 |
| Fennel seed oil | 0.1 |
| Thymol | 0.05 |
| Vanilla extract | 0.3 |
| PHMB | 0.02 |
| Other ingredients | |
| Water | 66.71 |
| Zemea | 4.0 |
| Alcohol | 10.0 |
| Pluronic F 127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |

In a specific non-limiting embodiment, an oral rinse (referred to as “Oral rinse A3.2”) is provided comprising the following:

| Main ingredients | % w /w |
|---|---|
| Cinnamon bark oil | 0.01 |
| Basil oil | 0.01 |
| Fennel seed oil | 0.1 |
| Thymol | 0.05 |
| Vanilla extract | 0.3 |
| PAPB | 0.02 |
| Other ingredients | |
| Water | 66.71 |
| Zemea | 4.0 |
| Alcohol | 10.0 |
| Pluronic F 127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |
| Tetrahydrocurcumin | 0.05 |

In a specific non-limiting embodiment, an oral rinse (referred to as “Oral rinse 17A.1”) is provided comprising the following:

| Main ingredients | % w/w |
|---|---|
| Tetrahydrocurcumin | 0.05 |
| Cinnamon bark oil | 0.01 |
| Basil oil | 0.01 |
| Fennel seed oil | 0.1 |
| Thymol | 0.05 |
| Vanilla extract | 0.3 |
| PHMB | 0.1 |
| Other ingredients | |
| Water | 76.73 |
| Zemea | 4.0 |
| Propylene glycol | 2.0 |
| Alcohol | 10.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |

In a specific non-limiting embodiment, an oral rinse (referred to as “Oral rinse 17A.2”) is provided comprising the following:

| Main ingredients | % w/w |
|---|---|
| Cinnamon bark oil | 0.01 |
| Basil oil | 0.01 |
| Fennel seed oil | 0.1 |
| Thymol | 0.05 |
| Vanilla extract | 0.3 |
| PAPB | al |
| Other ingredients | |
| Water | 64.63 |
| Zemea | 4.0 |
| Propylene glycol | 2.0 |
| Alcohol | 10.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |
| Tetrahydrocurcumin | 0.05 |

In a specific non-limiting embodiment, an oral rinse (referred to as “Oral rinse 17BCF-C.1”) is provided comprising the following:

| Main ingredients | % w/w |
|---|---|
| Cinnamon bark oil | 0.01 |
| Clove bud oil | 0.01 |
| Basil oil | 0.01 |
| Fennel seed oil | 0.1 |
| Thymol | 0.05 |
| Vanilla extract | 0.3 |
| PHMB | 0.1 |
| Other ingredients | |
| Water | 74.67 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |

In a specific non-limiting embodiment, an oral rinse (referred to as “Oral rinse 17BCF-C.2”) is provided comprising the following:

| Main ingredients | % w/w |
|---|---|
| Cinnamon bark oil | 0.01 |
| Clove bud oil | 0.01 |
| Basil oil | 0.01 |
| Fennel seed oil | 0.1 |
| Thymol | 0.05 |
| Vanilla extract | 0.3 |
| PAPB | 0.1 |
| Other ingredients | |
| Water | 74.67 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |

In a specific non-limiting embodiment, an oral rinse (referred to as “Oral rinse 17A-C.1”) is provided comprising the following:

| Main ingredients | % w/w |
|---|---|
| Tetrahydrocurcumin | 0.05 |
| Cinnamon bark oil | 0.01 |
| Clove bud oil | 0.01 |
| Basil oil | 0.01 |
| Fennel seed oil | 0.1 |
| Thymol | 0.05 |
| Vanilla extract | 0.3 |
| PHMB | 0.1 |
| Other ingredients | |
| Water | 64.62 |
| Zemea | 4.0 |
| Propylene glycol | 2.0 |
| Alcohol | 10.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |

In a specific non-limiting embodiment, an oral rinse (referred to as “Oral rinse 17A-C.2”) is provided comprising the following:

| Main ingredients | % w/w |
|---|---|
| Cinnamon bark oil | 0.01 |
| Clove bud oil | 0.01 |
| Basil oil | 0.01 |
| Fennel seed oil | 0.1 |
| Thymol | 0.05 |
| Vanilla extract | 0.3 |
| PAPB | 0.1 |
| Other ingredients | |
| Water | 64.67 |
| Zemea | 4.0 |
| Propylene glycol | 2.0 |
| Alcohol | 10.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |
| Tetrahydrocurcumin | 0.05 |

In a specific non-limiting embodiment, an oral rinse is provided with a general formula comprising the following range of ingredients

Oral Rinse

| Ingredient | % w/w |
|---|---|
| Zemea | 2-4.0 |
| Propylene glycol | 1-2.0 |
| Cinnamon bark oil | 0-0.05 |
| Basil oil | 0.01-0.05 |
| Peppermint oil | 0.05-0.1 |
| Plantasol | 0.0-0.5 |
| Water | 60-72 |
| Pluronic F-127 | 0.5-2.0 |
| Benzoic acid | 0.05-2.0 |
| Zinc salicylate | 0-0.2 |
| Sorbitol | 2.0-6.0 |
| Xylitol | 1-8.0 |
| Glycerin | 3-12.0 |
| Solubilizer 611674 | 0.25-0.5 |
| Biguanide | 0.03-0.12 |
| Thymol | 0.05-0.1 |
| Fennel seed oil | 0.1-0.3 |
| Vanilla oil | 0.1-03 |
| Menthol | 0.0-0.2 |
| Botanical extract (Vanilla/Lemon/Grape Fruit) | 0.1-0.6 |

In a specific non-limiting embodiment, an oral rinse (referred to as “PTFV1 Oral Rinse”) is provided comprising the following:

PTFV1 Oral Rinse

| Ingredient | % w/w |
|---|---|
| Zemea | 4.0 |
| Propylene glycol | 2.0 |
| Cinnamon bark oil | 0.01 |
| Basil oil | 0.01 |
| Peppermint oil | 0.05 |
| Plantasol | 0.5 |
| Water | 71.33 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| PHMB | 0.1 |
| Thymol | 0.1 |
| Fennel seed oil | 0.1 |
| Vanilla oil | 0.1 |

In a specific non-limiting embodiment, an oral rinse (referred to as “PTFV2 Oral Rinse”) is provided comprising the following:

PTFV2 Oral Rinse

| Ingredient | % w/w |
|---|---|
| Zemea | 4.0 |
| Propylene glycol | 2.0 |
| Cinnamon bark oil | 0.01 |
| Basil oil | 0.01 |
| Peppermint oil | 0.05 |
| Plantasol | 0.5 |
| Water | 71.37 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| PHMB | 0.06 |
| Thymol | 0.1 |
| Fennel seed oil | 0.1 |
| Vanilla oil | 0.1 |

In a specific non-limiting embodiment, an oral rinse (referred to as “PTFV-E1 Oral Rinse”) is provided comprising the following:

PTFV-E1 Oral Rinse

| Ingredient | % w/w |
|---|---|
| Zemea | 4.0 |
| Propylene glycol | 2.0 |
| Cinnamon bark oil | 0.01 |
| Basil oil | 0.01 |
| Peppermint oil | 0.05 |
| Plantasol | 0.5 |
| Water | 71.03 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| PHMB | 0.1 |
| Thymol | 0.1 |
| Fennel seed oil | 0.1 |
| Vanilla oil | 0.1 |
| Vanilla Extract | 0.3 |

In a specific non-limiting embodiment, an oral rinse (referred to as “PTFV-E2 Oral Rinse”) is provided comprising the following:

PTFV-E2 Oral Rinse

| Ingredient | % w/w |
|---|---|
| Zemea | 4.0 |
| Propylene glycol | 2.0 |
| Cinnamon bark oil | 0.01 |
| Basil oil | 0.01 |
| Peppermint oil | 0.05 |
| Plantasol | 0.5 |
| Water | 71.07 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| PHMB | 0.06 |
| Thymol | 0.1 |
| Fennel seed oil | 0.1 |
| Vanilla oil | 0.1 |
| Vanilla Extract | 0.3 |

In a specific non-limiting embodiment, an oral rinse (referred to as “CTFV Oral Rinse”) is provided comprising the following:

CTFV Oral Rinse

| Ingredient | % w/w |
|---|---|
| Zemea | 4.0 |
| Propylene glycol | 2.0 |
| Cinnamon bark oil | 0.01 |
| Basil oil | 0.01 |
| Peppermint oil | 0.05 |
| Plantasol | 0.5 |
| Water | 71.56 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.25 |
| Chlorhexidine | 0.12 |
| Thymol | 0.1 |
| Fennel seed oil | 0.1 |
| Vanilla oil | 0.1 |

In a specific non-limiting embodiment, an oral rinse (referred to as “CTFV-E Oral Rinse”) is provided comprising the following:

CTFV-E Oral Rinse

| Ingredient | % w/w |
|---|---|
| Zemea | 4.0 |
| Propylene glycol | 2.0 |
| Cinnamon bark oil | 0.01 |
| Basil oil | 0.01 |
| Peppermint oil | 0.05 |
| Plantasol | 0.5 |
| Water | 71.26 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.25 |
| Chlorhexidine | 0.12 |
| Thymol | 0.1 |
| Fennel seed oil | 0.1 |
| Vanilla oil | 0.1 |
| Vanilla Extract | 0.3 |

### 4.3 Topical Disinfectants

Disclosed herein are compositions that may be comprised in disinfectant compositions that may be used to topically to reduce the number and/or growth of microbes on the skin of a human or non-human animal. The disinfectant may be a wash solution, a lotion, a cream, or a splash and/or may be comprised in a wipe. These compositions described herein can be used in, for example, skin sanitizers, wound care products and other personal and health care products. This composition can also be used to enhance the activity of products containing preservative or low alcohol (1-21%). The compositions described herein can also be used in veterinary products, for example, in a cow teat disinfectant or a mastitis cream.

The disinfectant composition comprises the recited active agents and a suitable solvent. Non-limiting examples of suitable solvents include water, ethanol, glycerin, octoxyglycerin, and mixtures thereof.

Disclosed herein is a method of reducing the number and/or growth of microbes on the skin of a subject comprising applying to the skin a topical disinfectant composition as set forth herein. Microbes, the numbers and/or growth of which are reduced, include but are not limited to one or more of Staphylococcus aureus (including methicillin resistant Staphylococcus aureus), Staphylococcus epidermidis, Escherichia coli, Pseudomonas aeruginosa, Candida albicans, Aspergillus niger and Listeria monocytogenes.

In certain non-limiting embodiments, a topical antimicrobial/anti irritant/anti-inflammatory gel composition is provided comprising (a) one or more plant/fruit extract which may be a citrus extract, lemon extract, orange extract, grapefruit seed extract or grapefruit extract, vanilla extract, or a combination thereof (e.g. at a concentration of between about 0.15 and 0.6% w/w or between about 0.1% and 5% w/w); (b) one or more synthetic antimicrobial which may be a quaternary ammonium compound such as benzalkonium chloride, benzethonium chloride, or cetyl pyrinidium chloride, a biguanide such as chlorhexidine free base or chlorhexidine salt (e.g. chlorhexidine gluconate or chlorhexidine acetate) or polyhexamethylene biguanide, or a combination thereof (e.g. at a concentration between about 0.05% and 0.2% w/w or between about 0.2% and 5.0% w/w); (c) one or more essential oil or ingredient thereof which may be thymol, orange oil, rosemary oil, lemongrass oil, cinnamon oil, or a combination thereof (e.g. at a concentration between about 0.05 and 0.5% w/w or between about 0.5 and 4.0% w/w); (d) one or more botanical anti-irritant which may be aloe gel/extract (e.g. at a concentration between about 0.1 and 50% w/w), aloe leaf juice (e.g. at a concentration between about 0.5 and 2.0% w/w), bisabolol (e.g. at a concentration between about 0.05 and 2.0% w/w), an oat product such as beta glucan, oat kernel extract, or oat flour (e.g. at a concentration between about 0.2 and 3.0% w/w), and combinations thereof; (e) one or more anti-inflammatory agent which may be calendula oil, rosemary oil, a curcumin compound, pomegranate oil, Echinacea purpurea extract, portulaca extract, resveratrol, and combinations thereof (e.g. at a concentration between about 0 and 0.1% w/w or between about 0.1 and 0.5% w/w); (f) one or more alkanediol selected from the group consisting of propanediol, pentanediol, hexanediol, heptanediol, octanediol, decanediol, nonanediol and combinations thereof, at a concentration of between about 0 and 10% w/w, or between about 0 and 5% w/w, or between about 0.01 and 0.5% w/w and (g) an organic acid selected from the group consisting of benzoic acid, lactic acid, citric acid and combinations thereof, at a concentration of between about 0.01 and 1% w/w, or between about 0.05 and 0.5% w/w, or between about 0.25 and 2.0% w/w. Additional optional components of such compositions may include (i) an antioxidant such as tocopherol acetate or ascorbic acid; (ii) an emollient which may be 1,3-propanediol, butylene glycol, propylene glycol, glycerine, octoxyglycerine, or a combination thereof (e.g. at a concentration of between about 0.5 and 6.0% w/w or between about 3.0 and 30.0% w/w); and/or (iii) a gelling agent which may be hydroxypropylmethyl cellulose, hydroxy propyl methyl cellulose stearoxy ether (Sangelose), chitosan, pyrrolidone carboxylate (Kytamer) (e.g. at a concentration of between about 0.01 and 1.0% w/w)

In certain non-limiting embodiments, the topical composition described herein comprises: (i) a plant extract selected from the group consisting of vanilla extract, lemon extract, citrus extract, grapefruit seed extract and combinations thereof at a concentration of between about 0.05 and 1% w/w, or between about 0.1 and 0.6% w/w;

(ii) polyhexamethylene biguanide (PHMB), polyaminopropyl biguanide (PAPB) or combinations thereof at a concentration of between about 0.001 and 5% w/w, or between about 0.005 and 1% w/w, or between about 0.01 and 0.3% w/w; and

(iii) one or more essential oil or active ingredient thereof selected from the group consisting of lemongrass oil, lemon oil, fennel seed oil, orange oil, basil oil, cinnamon oil, thyme oil, clove oil, pine oil, cedar wood oil, thymol, farnesol, bisabolol, cinnamaldehyde, cinnamylacetic ester, cinnamic acid, ethyl cinnamate or combinations thereof, at a concentration of between about 0.005 and 1% w/w, or between about 0.01 and 0.5% w/w.

In certain embodiments, the composition comprises vanilla extract, lemon extract or combinations thereof, and does not comprise any other citrus extract.

In certain non-limiting embodiments, the composition described herein further comprises: (iv) one or more alkanediol selected from the group consisting of propanediol, pentanediol, hexanediol, heptanediol, octanediol, decanediol, nonanediol and combinations thereof, at a concentration of between about 0 and 10% w/w, or between about 0 and 5% w/w, or between about 0.01 and 0.5% w/w;

(v) an organic acid selected from the group consisting of benzoic acid, lactic acid, citric acid and combinations thereof, at a concentration of between about 0.01 and 1% w/w, or between about 0.05 and 0.5% w/w;

(vi) a solvents which enhances the solubility of the ingredients of the composition, selected from the group consisting of alcohol (between about 0 and 80% w/w), phenyl ethanol (between about 0 and 1.0% w/w), a mixture of PEG-40 hydrogenated castor oil, Trideceth-9, propylene glycol and water (between about 0 and 2% w/w), a mixture of PEG-40 hydrogenated castor oil, Trideceth-9 and water (between about 0 and 2% w/w), caprylyl capryl glucoside (Plantasol) (between about 0 and 2% w/w), and combinations thereof;

(vii) one or more anti-irritant selected from the group consisting of zinc salicylate, basil oil, brahmi extract, red sandal wood extract, curcumin compound, aloe, bisabolol, rosemary oil, chalmogra oil, oat powder and/or extract, beta glucan, chitosan, calendula oil, portulaca extract, witch hazel extract, pomegranate oil, at a concentration of between about 0.05 and 5% w/w, or between about 0.1 and 2% w/w.

In certain non-limiting embodiments, the composition described herein comprises: (i) a plant extract selected from the group consisting of vanilla extract, lemon extract and combinations thereof at a concentration of between about 0.1 and 0.6% w/w; (ii) polyhexamethylene biguanide (PHMB) at a concentration of between about 0.01 and 0.3% w/w; and (iii) one or more essential oil or active ingredient thereof selected from the group consisting of thyme oil, cinnamon oil, thymol, cinnamaldehyde, cinnamylacetic ester, cinnamic acid, ethyl cinnamate or combinations thereof, at a concentration of between about 0.01 and 0.5% w/w.

“Hand sanitizers” is a term used herein to refer to skin sanitizers, which also may be used as topical disinfectants for skin of non-human animals.

In certain non-limiting embodiments, hand sanitizer products are provided as follows.

In certain non-limiting embodiments, a hand sanitizer foam is provided comprising the following:

| Ingredients | % w/w |
|---|---|
| Water | 86.425 |
| Hydroxypropyl methyl- | 0.025 |
| cellulose stearoxy ether (Sangelose) | |
| Solubulizer (Peg-40 hydrogented | 2.0 |
| Castor oil, Trideceth 9, water), | |
| Plantasol | 3.5 |
| Octanediol | 1.0 |
| Phenyl ethanol | 0.3 |
| Lemongrass oil | 0.1 |
| Aloe leaf juice | 1.0 |
| BAC | 0.13 |
| Thymol | 0.07 |
| Cinnamon bark oil | 0.1 |
| Zemea | 1.0 |
| Propylene glycol | 1.0 |
| Butyl ene glycol | 1.0 |
| Cetyl pyridium chloride | 0.25 |
| Citrus extract | 0.3 |
| Bisabolol | 0.05 |
| Dow corning fluid 193 C | 0.25 |
| Dow corning cosmetic wax 2501 | 0.75 |
| Isododecane and isononanoate | 0.25 |
| (Clerocast 550) 2-methyl-1, 3-propanediol | 0.50 |
| (Solidol MP-D) | |

In certain non-limiting embodiments, a hand sanitizer foam is provided comprising the following:

| Ingredients | % w/w |
|---|---|
| Water | 86.425 |
| Hydroxypropyl methyl- | 0.025 |
| cellulose stearoxy ether (Sangelose) | |
| Solubulizer (Peg-40 hydrogented | 2.0 |
| Castor oil, Trideceth 9, water), | |
| Plantasol | 3.0 |
| Lemon extract | 0.5 |
| Octanediol | 1.0 |
| Phenyl ethanol | 0.3 |
| Lemongrass oil | 0.1 |
| Aloe leaf juice | 1.0 |
| BAC | 0.13 |
| Thymol | 0.07 |
| Cinnamon bark oil | 0.1 |
| Zemea | 1.0 |
| Propylene glycol | 1.0 |
| Butylene glycol | 1.0 |
| Cetyl pyridium chloride | 0.25 |
| Citrus extract | 0.3 |
| Bisabolol | 0.05 |
| Dow corning fluid 193 C | 0.25 |
| Dow corning cosmetic wax 2501 | 0.75 |
| Isododecane and isononanoate | 0.25 |
| (Clerocast 550) | |
| 2-methyl-1, 3-propanediol | 0.50 |
| (Solidol MP-D) | |

In certain non-limiting embodiments, a band sanitizer foam is provided comprising the following ranges of ingredients:

| Ingredients | % w/w |
|---|---|
| Water | 40-98 |
| Hydroxypropyl methyl- | 0.01-0.5 |
| cellulose stearoxy ether (Sangelose) | |
| Solubulizer (Peg-40 hydrogented | 0.25-2.0 |
| Castor oil, Trideceth 9, water), | |
| Plantasol | 0.5-4.0 |
| Lemon extract | 0.1-1.0 |
| Octanediol | 0.2-4.0 |
| Phenyl ethanol | 0.1-2.0 |
| Lemongrass oil | 0.05-2.0 |
| Aloe leaf juice | 0.1-2.0 |
| BAC | 0.05-2.0 |
| PHMB | 0.01-1.0 |
| Thymol | 0.01-1.0 |
| Cinnamon oil | 0.05-1.0 |
| Dipropylene glycol | 0.5-2.0 |
| Cetyl pyridium chloride | 0.05-0.50 |
| Citrus extract | 0.0-1.0 |
| Bisabolol | 0.01-1.0 |
| Ethoxy diglycol(Edicol) | 0.5-3.0 |
| Dow corning cosmetic wax 2501 | 0.2-2.0 |
| Isododecane and isononanoate | 0.1-1.0 |
| (Clerocast 550) | |

In a specific non-limiting embodiment, a hand sanitizer foam is provided comprising the following concentrations within the ranges set forth in the preceding paragraph:

| Ingredients | % w/w |
|---|---|
| Water | 88.865 |
| Hydroxypropyl methyl- | 0.025 |
| cellulose stearoxy ether (Sangelose) | |
| Solubulizer (PEG-40 hydrogented | 2.0 |
| Castor oil, Trideceth 9, water) | |
| Plantasol | 2.0 |
| Lemon extract | 0.5 |
| Octanediol | 1.0 |
| Phenyl ethanol | 0.3 |
| Lemongrass oil | 0.1 |
| Aloe leaf juice | 1.0 |
| BAC | 0.13 |
| PHMB | 0.06 |
| Thymol | 0.07 |
| Cinnamon oil | 0.1 |
| Dipropylene glycol | 1.0 |
| Cetyl pyridium chloride | 0.25 |
| (Non-lemon) Citrus extract | 0.3 |
| Bisabolol | 0.05 |
| Ethoxy diglycol(Edicol) | 1.0 |
| Dow corning cosmetic wax 2501 | 0.75 |
| Isododecane and isononanoate | 0.50 |
| (Clerocast 550) | |

In certain non-limiting embodiments, a botanical hand sanitizer foam is provided comprising the following ranges of ingredients:

| Ingredients | % w/w |
|---|---|
| Water | 40-98 |
| Hydroxypropyl methyl- | 0.01-0.5 |
| cellulose stearoxy ether (Sangelose) | |
| Solubulizer | 0.5-4.0 |
| (Peg-40 hydrogented | |
| Castor oil, Trideceth 9, water) | |
| Plantasol | 0.5-4.0 |
| Lemon extract | 0.1-1.0 |
| Alkanediol | 0.0-2.0 |
| Phenyl ethanol | 0.1-2.0 |
| Lemongrass oil | 0.05-2.0 |
| Aloe leaf juice | 0.1-2.0 |
| BAC | 0.05-2.0 |
| PHMB | 0.01-1.0 |
| Thymol | 0.01-1.0 |
| Cinnamon oil | 0.05-1.0 |
| Cetyl pyridium chloride | 0.05-0.50 |
| (Non-lemon) Citrus extract | 0.0-1.0 |
| Bisabolol | 0.01-1.0 |
| Ethoxy diglycol | 0.5-3.0 |
| 2-methyl-1, 3-propanediol | 0.2-2.0 |
| (Solidol MP-D) | |
| Dow corning cosmetic wax 2501 | 0.2-2.0 |
| Isododecane and isononanoate | 0.0-1.0 |
| (Clerocast 550) | |
| Dimethicone | 0.0-3.0 |

In a specific non-limiting embodiment, a hand sanitizer foam is provided comprising the following concentrations within the ranges set forth in the preceding paragraph:

| Ingredients | % w/w |
|---|---|
| Water | 91.395 |
| Hydroxypropyl methyl- | 0.025 |
| cellulose stearoxy ether (Sangelose) | |
| Solubulizer (Peg-40 hydrogented | 1.75 |
| Castor oil, Trideceth 9, water) | |
| Plantasol | 1.5 |
| Lemon extract | 0.3 |
| Vanilla extract | 0.3 |
| Phenyl ethanol | 0.3 |
| Lemongrass oil | 0.05 |
| Aloe leaf juice | 1.0 |
| BAC | 0.13 |
| PHMB | 0.10 |
| Thymol | 0.05 |
| Cinnamon oil | 0.05 |
| Cetyl pyridium chloride | 0.25 |
| Citrus extract | 0.5 |
| Bisabolol | 0.05 |
| Ethoxy diglycol | 0.5 |
| 2-methyl-1, 3-propanediol | 0.50 |
| (Solidol MP-D) | |
| Dow corning cosmetic wax 2501 | 0.75 |
| Isododecane and isononanoate | 0.50 |
| (Clerocast 550) | |

In a specific non-limiting embodiment, a hand sanitizer foam is provided comprising the following concentrations within the ranges set forth in paragraph preceding the paragraph immediately above:

| Ingredients | % w/w |
|---|---|
| Water | 89.445 |
| Hydroxypropyl methyl- | 0.025 |
| cellulose stearoxy ether (Sangelose) | |
| BAC | 0.13 |
| PHMB | 0.1 |
| Octanediol | 1.0 |
| Rosemary oil | 0.1 |
| Phenyl ethanol | 0.3 |
| Lactic acid | 0.1 |
| Cinnamon bark oil | 0.1 |
| Lemon grass oil | 0.05 |
| Thymol | 0.05 |
| Lemon extract | 0.5 |
| Bisabolol | 0.1 |
| Cetyl pyridinium chloride | 0.25 |
| Aloe leaf juice | 1.0 |
| Solubulizer (Peg-40 hydrogented | 1.75 |
| Castor oil, Trideceth 9, water) | |
| Plantasol | 1.5 |
| Ethoxy diglycol | 0.5 |
| 2-methyl-1, 3-propanediol | 0.75 |
| (Solidol MP-D) | |
| Dow corning cosmetic wax 2501 | 0.75 |
| Isododecane and isononanoate | 0.50 |
| (Clerocast 550) | |
| Dimethicone(Silicon KF 6043) | 1.0 |

In certain non-limiting embodiments, a hand sanitizer foam is provided comprising the following ranges of ingredients:

| Ingredients | % w/w |
|---|---|
| Water | 70-87.76 |
| Hydroxypropyl methyl- | 0.025-0.4 |
| cellulose stearoxy ether (Sangelose) | |
| Vanilla extract | 0.1-0.6 |
| Octanediol | 0.5-1.0 |
| Phenyl ethanol | 0.3-0.7 |
| Lemongrass oil | 0.1-0.5 |
| Orange oil | 0.1-0.5 |
| Aloe leaf juice | 2.0-10.0 |
| Benzalkonium chloride | 0.01-0.12 |
| CHG (20%) | 0.25-1.0 |
| Thymol | 0.03-0.1 |
| Basil oil | 0.01-0.1 |
| Fennel oil | 0.1-0.3 |
| Vanilla oil | 0.1-0.3 |
| Lactic acid | 0.1-0.3 |
| Zemea | 1.0-5.0 |
| Butylene glycol | 0-5.0 |
| Bisabolol | 0.05-0.2 |
| Calendula extract | 0-0.5 |
| Dow corning fluid 193 C | 0.25-0.5 |
| Solubulizer (Peg-40 hydrogented | 1-3.0 |
| Castor oil, Trideceth 9, water) | |
| Plantasol | 0.25-4.0 |
| Bioplex | 0.25-1.0 |
| Shin Etsu Silicon KF 6004 | 0.025-0.5 |
| Adjust pH to 4.0-4.2 | |

In certain non-limiting embodiments, a hand sanitizer foam is provided comprising the following ingredients:

| Ingredients | % w/w |
|---|---|
| Water | 87.76 |
| Hydroxypropyl methyl- | 0.025 |
| cellulose stearoxy ether (Sangelose) | |
| Vanilla extract | 0.3 |
| Octanediol | 0.8 |
| Phenyl ethanol | 0.3 |
| Lemongrass oil | 0.2 |
| Orange oil | 0.1 |
| Aloe leaf juice | 2.0 |
| BAC | 0.12 |
| CHG (20%) | 1.0 |
| Thymol | 0.1 |
| Basil oil | 0.05 |
| Fennel oil | 0.1 |
| Vanilla oil | 0.1 |
| Lactic acid | 0.1 |
| Zemea | 1.0 |
| Butylene glycol | 1.0 |
| Bisabolol | 0.05 |
| Dow corning fluid 193 C | 0.25 |
| Solubulizer (Peg-40 hydrogented | 2.0 |
| Castor oil, Trideceth 9, water) | |
| Plantasol | 2.0 |
| Bioplex | 0.5 |
| Shin Etsu Silicon KF 6004 | 0.25 |
| Adjust pH to 4.0-4.2 | |

In a specific non-limiting embodiment, a hand sanitizer lotion is provided comprising the following concentrations:

| Ingredients | % w/w |
|---|---|
| Water | 84.25 |
| Hydroxypropyl methyl- | 0.2 |
| cellulose stearoxy ether (Sangelose) | |
| Cetyl pyridinium chloride | 0.25 |
| Propylene glycol | 1.0 |
| Glycerin | 3.0 |
| Heat 50-60° C. | |
| Incroquat TMS | 1.5 |
| Polowax NF | 0.5 |
| Stearyl alcohol | 0.5 |
| Zinc oxide | 0.3 |
| Silicone fluid (Dimethicone200/350) | 2.0 |
| Aloe juice | 1.0 |
| Bisabolol | 0.05 |
| Grape fruit seed extract | 0.3 |
| Phenyl Ethanol | 0.3 |
| Thymol | 0.07 |
| BAC | 0.13 |
| Lemongrass oil | 0.05 |
| Pomegranate oil | 0.4 |
| Almond oil | 1.0 |
| Zemea | 1.0 |
| Lactic acid | 0.1 |
| Octanediol | 1.0 |
| Cinnamon bark oil | 0.1 |
| Dow corning 2501 | 0.75 |
| Clerocast 550 | 0.25 |
| Adjust pH to 4.0-4.5 | |

In a specific non-limiting embodiment, a hand sanitizer lotion is provided comprising the following concentrations:

| Ingredients | % w/w |
|---|---|
| Water | 83.81 |
| Hydroxypropyl methyl-cellulose | 0.2 |
| stearoxy ether (Sangelose) | |
| Cetyl pyridinium chloride | 0.25 |
| Propylene glycol | 1.0 |
| Glycerin | 3.0 |
| Incroquat TMS | 1.5 |
| Polowax NF | 0.5 |
| Stearyl alcohol | 0.5 |
| Zinc oxide | 0.3 |
| Silicone fluid (Dimethicone200/350) | 2.0 |
| Aloe juice | 1.0 |
| Bisabolol | 0.05 |
| Grape fruit seed extract | 0.25 |
| Lemon extract | 0.25 |
| Phenyl Ethanol | 0.3 |
| Thymol | 0.05 |
| BAC | 0.13 |
| PHMB | 0.06 |
| Lemongrass oil | 0.05 |
| Pomegranate oil | 0.4 |
| Almond oil | 1.0 |
| 2-methyl-1, 3-propanediol | 1.0 |
| (Solidol) | |
| Lactic acid | 0.1 |
| Octanediol | 1.0 |
| Cinnamon leaf oil | 0.05 |
| Dow corning 2501 | 0.75 |
| Clerocast 550 | 0.50 |
| Adjust pH to 4.0-4.5 | |

In a specific non-limiting embodiment, a hand sanitizer lotion is provided comprising the following concentrations:

| Ingredients | % w/w |
|---|---|
| Water | 84.07 |
| Hydroxypropyl methyl-cellulose | 0.2 |
| stearoxy ether (Sangelose) | |
| BAC | 0.13 |
| PHMB | 0.1 |
| Cetyl pyridinium chloride | 0.25 |
| Propylene glycol | 1.0 |
| Glycerin | 3.0 |
| Incroquat TMS | 1.5 |
| Polowax NF | 0.5 |
| Stearyl alcohol | 0.5 |
| Zinc oxide | 0.3 |
| Silicone fluid (Dimethicone200/350) | 2.0 |
| Aloe juice | 1.0 |
| Bisabolol | 0.05 |
| Lemon extract | 0.50 |
| Thymol | 0.05 |
| Lemongrass oil | 0.05 |
| Pomegranate oil | 0.4 |
| Almond oil | 1.0 |
| 2-methyl-1, 3-propanediol | 1.0 |
| (Solidol) | |
| Lactic acid | 0.1 |
| Octanediol | 1.0 |
| Cinnamon leaf oil | 0.05 |
| Dow corning 2501 | 0.75 |
| Clerocast 550 | 0.50 |
| Adjust pH to 4.0-4.5 | |

In a specific non-limiting embodiment, a hand sanitizer lotion is provided comprising the following:

| Ingredients | % w/w |
|---|---|
| Water | 85.19 |
| Hydroxypropyl methyl-cellulose | 0.2 |
| stearoxy ether (Sangelose) | |
| BAC | 0.13 |
| PHMB | 0.1 |
| Glycerin | 2.0 |
| Incroquat TMS | 1.5 |
| Polowax NF | 1.0 |
| Stearyl alcohol | 0.8 |
| Zinc oxide | 0.4 |
| Aloe juice | 1.0 |
| Bisabolol | 0.05 |
| Zemea | 1.0 |
| Octanediol | 1.0 |
| Cinnamon bark oil | 0.05 |
| Lemon grass oil | 0.05 |
| Thymol | 0.03 |
| Lemon extract | 0.5 |
| Rosemary oil | 0.1 |
| Pomogranite oil | 0.3 |
| Lactic acid | 0.1 |
| Almond oil | 1.5 |
| Silicone 250/300 | 2.0 |
| Silicon KSG 210 | 1.0 |

In a specific non-limiting embodiment, a hand sanitizer lotion is provided comprising the following range of ingredients.

| Ingredients | % w/w |
|---|---|
| Water | 65-78 |
| Hydroxypropyl methyl- | 0.1-0.4 |
| cellulose stearoxy ether (Sangelose) | |
| Cetyl pyridinium chloride | 0-0.25 |
| Propylene glycol | 0-1.0 |
| Glycerin | 1-5.0 |
| Oat powder | 1-3.0 |
| Incroquat TMS | 2-4 |
| Polowax NF | 0.5-2 |
| Stearyl alcohol | 0.5-1.0 |
| Zinc oxide | 0.3-0.6 |
| Silicone fluid (Dimethicone200/350) | 0.5-2.0 |
| Aloe Vera gel/juice | 2-10 |
| Grape fruit seed extract | 0.3-1.0 |
| Phenyl Ethanol | 0.3-0.7 |
| Thymol | 0.03-0.1 |
| Vanilla oil | 0.1-0.3 |
| Fennel oil | 0.1-0.3 |
| Basil oil | 0.02-0.3 |
| Benzalkonium chloride | 0.01-0.12 |
| CHG (20%) | 0.25-1.0 |
| Lemongrass oil | 0.1-0.5 |
| Orange oil | 0.1-0.3 |
| Pomegranate oil/Extract | 0.1-0.4 |
| Calendula extract | 0.3-0.5 |
| Almond oil | 1.0-3.0 |
| Zemea | 1.0-3.0 |
| Lactic acid | 0.1-0.3 |
| Octanediol | 0.5-1.0 |
| Plantasol | 0.25-5.0 |
| Solubulizer 611674 | 0.5 2.0 |
| Adjust pH to 4.0-4.2 | |

In a specific non-limiting embodiment, a hand sanitizer lotion is provided comprising the following:

| Ingredients | % w/w |
|---|---|
| Water | 74.455 |
| Hydroxypropyl methyl- | 0.2 |
| cellulose stearoxy ether (Sangelose) | |
| Cetyl pyridinium chloride | 0.125 |
| Propylene glycol | 1.0 |
| Glycerin | 3.0 |
| Oat Powder | 1.0 |
| Incroquat TMS | 3.0 |
| Polowax NF | 1.5 |
| Stearyl alcohol | 1.0 |
| Zinc oxide | 0.3 |
| Silicone fluid (Dimethicone200/350) | 2.0 |
| Aloeveragel/juice | 2.0 |
| Grape fruit seed extract | 0.3 |
| Phenyl Ethanol | 0.3 |
| Thymol | 0.1 |
| Vanilla oil | 0.1 |
| Fennel oil | 0.1 |
| Basil oil | 0.05 |
| Benzalkoniuin chloride | 0.12 |
| CHG (20%) | 1.0 |
| Lemongrass oil | 0.2 |
| Orange oil | 0.1 |
| Pomegranate oil/Extract | 0.4 |
| Calendula extract | 0.5 |
| Almond oil | 1.0 |
| Zemea | 1.0 |
| Lactic acid | 0.1 |
| Octanediol | 0.8 |
| Plantasol | 2.0 |
| Solubulizer 611674 | 2.0 |
| Adjust pH to 4.0-4.2 | |

In certain non-limiting embodiments, an anti-inflammatory, anti-irritant topical gel is provided comprising the following ranges of ingredients:

| Ingredients | % w/w |
|---|---|
| Water | 15-40 |
| Oat Powder | 0.5-5.0 |
| Tetrahydrocurcumanoid | 0.05-1.0 |
| PHMB | 0.05-1.0 |
| Lemon/citrus extract | 0.05-2.0 |
| Sym glucan (Beta glucan) | 0.0-3.0 |
| Dragocalm (Oat Kernel extract) | 0.1-2.0 |
| Chitidine 222 (Chitosan succinate) | 0.5-3.0 |
| Witch hazel extract | 0.0-2.0 |
| Portulaca extract | 0.0-2.0 |
| Propylene glycol | 2-20 |
| Octanediol | 0.1-2.0 |
| Hydroxypropyl methyl- | 0.00-0.5 |
| cellulose stearoxy ether (Sangelose) | |
| Aloe Gel | 20-70 |
| Petrolatum | 5-40 |
| Pomegranate oil | 0.05-2.0 |
| Cinnamon oil | 0.05-1.0 |
| Lemon grass oil | 0.05-1.0 |
| Rosemary oil | 0.05-1.0 |
| Tocopherol | 0.1-2.0 |
| Camphor | 0-0.5 |
| Natural emulsifier | 0.0-2.0 |
| (Glyceryl oleate citrate, | |
| Caprylic/capric triglyceride) | |

In a specific non-limiting embodiment, an anti-inflammatory, anti-irritant topical gel is provided comprising the following concentrations within the ranges set forth in the preceding paragraph:

| Ingredients | % w/w |
|---|---|
| Water | 22.9 |
| Oat Powder | 1.5 |
| Tetrahydrocurcumanoid | 0.1 |
| PHMB | 0.1 |
| Lemon/citrus extract | 0.15 |
| Sym glucan(Beta glucan) | 0.75 |
| Dragocalm(Oat Kernel extract) | 0.5 |
| Chitidine 222(Chitosan succinate) | 1.5 |
| Witch hazel extract | 0.5 |
| Propylene glycol | 10 |
| Octanediol | 0.8 |
| Hydroxypropyl methyl- | 0.1 |
| cellulose stearoxy ether (Sangelose) | |
| Aloe Gel | 40 |
| Petrolatum | 20 |
| Pomegranate oil | 0.3 |
| Cinnamon oil | 0.1 |
| Lemon grass oil | 0.1 |
| Rosemary oil | 0.1 |
| Tocopherol | 0.5 |

In a specific non-limiting embodiment, an anti-inflammatory, anti-irritant topical gel is provided comprising the following:

| Ingredients | % w/w |
|---|---|
| Water | 11.9 |
| Oat Powder | 1.5 |
| Tetrahydrocurcumanoid | 0.1 |
| PHMB | 0.1 |
| Lemon/citrus extract | 0.15 |
| Dragocalm (Oat Kernel extract) | 0.5 |
| Chitidine 222 (Chitosan succinate) | 1.5 |
| Portulaca extract | 0.75 |
| Propylene glycol | 10 |
| Octanediol | 0.8 |
| Hydroxypropyl methyl- | 0.1 |
| cellulose stearoxy ether (Sangelose) | |
| Aloe Gel | 40 |
| Petrolatum | 30 |
| Pomegranate oil | 0.3 |
| Cinnamon oil | 0.1 |
| Lemon grass oil | 0.1 |
| Rosemary oil | 0.1 |
| Natural Emulsifier (Symrise Dracorin) | 2.0 |

In a specific non-limiting embodiment, an anti-inflammatory, anti-irritant topical gel is provided comprising the following ingredients:

| Ingredients | % w/w |
|---|---|
| Water | 10.9 |
| Oat Powder | 1.5 |
| Tetrahydrocurcumanoid | 0.1 |
| PHMB | 0.1 |
| Lemon/citrus extract | 0.15 |
| Sym glucan (Beta glucan) | 0.75 |
| Dragocalm (Oat Kernel extract) | 0.5 |
| Chitidine 222 (Chitosan succinate) | 1.5 |
| Portuluca extract | 0.5 |
| Propylene glycol | 10 |
| Octanediol | 0.8 |
| Hydroxypropyl methyl- | 0.1 |
| cellulose stearoxy ether (Sangelose) | |
| Aloe Gel | 40 |
| Natutal petrolatum | 30 |
| Pomegranate oil | 0.3 |
| Cinnamon oil | 0.1 |
| Lemon grass oil | 0.1 |
| Rosemary oil | 0.1 |
| Tocopherol | 0.5 |
| Natural emulsifier | 2.0 |
| (Glyceryl oleate citrate, Caprylic/ | |
| capric triglyceride) | |

In certain non-limiting embodiments, a topical wound-healing gel is provided comprising the following ranges of ingredients:

| Ingredients | % w/w |
|---|---|
| Water | 15-40 |
| Oat Powder | 0.5-5.0 |
| Tetrahydrocurcumanoid | 0.05-1.0 |
| PHMB | 0.05-1.0 |
| Sym glucan (Beta glucan) | 0.1-3.0 |
| Dragocalm (Oat Kernel extract) | 0.1-2.0 |
| Chitidine 222 (Chitosan succinate) | 0.5-3.0 |
| Portulaca extract | 0.25-0.5 |
| Propylene glycol | 2-20 |
| Hydroxypropyl methyl- | 0-3.0 |
| cellulose stearoxy ether (Sangelose) | |
| Aloe Gel | 5-50 |
| Petrolatum | 5-20 |
| Tocopherol | 0.1-2.0 |

Said gel can be applied on the wound directly and/or incorporated in a wound dressing.

In a specific non-limiting embodiment, a topical wound-healing gel is provided comprising the following concentrations within the ranges set forth in the preceding paragraph:

| Ingredients | % w/w |
|---|---|
| Water | 34.1 |
| Oat Powder | 1.5 |
| Tetrahydrocurcumanoid | 0.1 |
| PHMB | 0.3 |
| Sym glucan (Beta glucan) | 0.75 |
| Dragocalm (Oat Kernel extract) | 0.5 |
| Chitidine 222 (Chitosan succinate) | 1.5 |
| Portulaca extract | 0.25 |
| Propylene glycol | 10 |
| Hydroxypropyl methyl- | 0.5 |
| cellulose stearoxy ether (Sangelose) | |
| Aloe Gel | 30 |
| Petrolatum | 20 |
| Tocopherol | 0.5 |

Said gel can be applied on the wound directly and/or incorporated in a wound dressing.

In a specific non-limiting embodiment, a botanical hand disinfectant soap is provided comprising the following concentrations:

| Ingredients | % w/w |
|---|---|
| Water | 83.73 |
| Polyox N60K | 0.3 |
| Pluronic surfactant F 87 | 1.0 |
| Cocoamido propyl betaine | 5.0 |
| Benzalkanoium chloride | 0.13 |
| Chlorhexidine gluconate (CHG) | 0.2 |
| Incromine Oxide-L | 1.0 |
| Glucopon 215 U P | 1.0 |
| Sugarnate 60 | 2.0 |
| Surfactant(Plantasol) | 1.5 |
| Lemon grass oil | 0.1 |
| Lactic acid | 0.4 |
| Phenyl ethanol | 0.5 |
| Phenoxy ethanol | 0.5 |
| Citrus extract | 03 |
| Aloe Juice | 0.5 |
| Glycerin | 1.0 |
| Cinnamon leaf oil | 0.07 |
| Thymol | 0.07 |
| Octanediol | 0.7 |

In certain non-limiting embodiments, a hand disinfectant soap (referred to as “SynBotanica-1”) is provided comprising the following ranges of ingredients:

| Ingredients | % w/w |
|---|---|
| Water | 55-80 |
| Polyox N60K | 0.1-0.3 |
| Polyquatemium 10 | 0.1-0.3 |
| Pluronic surfactant F 87 | 1-2 |
| Surfactant (Purasil Mild) | 0.0-1.0 |
| Cocoamido propyl betaine | 5-8 |
| Citric acid | 0.1-0.5 |
| Benzalkanoium chloride | 0.1-0.13 |
| PHMB | 0.06-0.3 |
| Alcohol (SDA40B) | 5-20 |
| Lemon grass oil | 0.05-0.5 |
| Phenoxy ethanol | 0.5-1.0 |
| Lemon extract | 0.3-0.6 |
| Aloe Juice | 0.5-2.0 |
| Glycerin | 0.5-3.0 |
| Octanediol | 0.5-1.0 |
| Cinnamon leaf oil | 0.03-0.1 |
| pH 4.0 | |

In a specific non-limiting embodiment, a hand disinfectant soap (referred to as “SynBotanica-1A”) is provided comprising the following concentrations within the ranges set forth in the preceding paragraph:

| Ingredients | % w/w |
|---|---|
| Water | 75.6 |
| Polyox N60K | 0.2 |
| Polyquaternium 10 | 0.2 |
| Pluronic surfactant F 87 | 2.0 |
| Cocoamido propyl betaine | 7.0 |
| Citric acid | 0.5 |
| Benzalkanoium chloride | 0.13 |
| PHMB | 0.1 |
| Alcohol (SDA40B) | 10.0 |
| Lemon grass oil | 0.2 |
| Phenoxy ethanol | 0.7 |
| Lemon extract | 0.6 |
| Aloe Juice | 1.0 |
| Glycerin | 1.0 |
| Octanediol | 0.7 |
| Cinnamon leaf oil | 0.07 |
| pH 4.0 | |

In certain non-limiting embodiments, a hand disinfectant soap (referred to as “SynBotanica-2”) is provided comprising the following ranges of ingredients:

| Ingredients | % w/w |
|---|---|
| Benzalkanoium chloride | 0.1-0.13 |
| Cosmocil | 0.3-1.5 |
| Lemon extract | 0.3-0.6 |
| Lemon grass oil | 0.05-0.5 |
| Thymol | 0.03-0.1 |
| Water | 55-76 |
| Polyox N60K | 0.1-0.3 |
| Polyquaternium 10 (ucare JR 30) | 0.1-0.3 |
| Pluronic surfactant F 87 | 1-2 |
| Cocoamido propyl betaine | 5-8 |
| Citric acid | 0.1-0.5 |
| Alcohol (SDA40B) | 5-20 |
| Phenoxy ethanol | 0.5-1.0 |
| Aloe Juice | 0.5-2.0 |
| Glycerin | 0.5-3.0 |
| Solubulizer (611624) | 0.3-2.0 |
| Plantasol | 1-2.5 |
| Octanediol | 0.5-1.0 |
| Cinnamon leaf oil | 0.03-0.1 |
| Dow corning Siliocone fluid | 0.5-2.0 |
| pH 4.0 | |

In a specific non-limiting embodiment, a hand disinfectant soap (referred to as “SynBotanica-2A”) is provided comprising the following concentrations within the ranges set forth in the preceding paragraph:

| Ingredients | % w/w |
|---|---|
| Benzalkanoium chloride | 0.13 |
| Cosmocil | 0.5 |
| Lemon extract | 0.6 |
| Lemon grass oil | 0.2 |
| Thymol | 0.05 |
| Water | 71.65 |
| Polyox N60K | 0.2 |
| Polyquaternium 10 (ucare JR 30) | 0.2 |
| Pluronic surfactant F 87 | 2.0 |
| Cocoamido propyl betaine | 7.0 |
| Citric acid | 0.5 |
| Alcohol (SDA40B) | 10.0 |
| Phenoxy ethanol | 0.7 |
| Aloe Juice | 1.0 |
| Glycerin | 1.0 |
| Solubulizer (611624) | 0.5 |
| Plantasol | 2.0 |
| Octanediol | 0.7 |
| Cinnamon leaf oil | 0.07 |
| Dow corning Siliocone fluid | 1.0 |
| pH 4.0 | |

In certain non-limiting embodiments, a hand disinfectant soap (referred to as “SynBotanica-3”) is provided comprising the following ranges of ingredients:

| Ingredients | % w/w |
|---|---|
| Benzalkanoium chloride | 0.1-0.13 |
| Cosmocil | 0.3-1.5 |
| Lemon extract | 0.3-0.6 |
| Lemon grass oil | 0.05-0.5 |
| Thymol | 0.03-0.1 |
| Water | 55-76 |
| Polyox N60K | 0.1-0.3 |
| Polyquaternium 10 (ucare JR 30) | 0.1-0.3 |
| Pluronic surfactant F 87 | 1-2 |
| Cocoamido propyl betaine | 5-8 |
| Citric acid | 0.1-0.5 |
| Natural Surfactant(Purasil Mild) | 0.0-2.0 |
| Alcohol (SDA40B) | 5-20 |
| Phenoxy ethanol | 0.5-1.0 |
| Aloe Juice | 0.5-2.0 |
| Glycerin | 0.5-3.0 |
| Solubulizer(611624) | 0.3-2.0 |
| Plantasol | 1-2.5 |
| Octanediol | 0.5-1.0 |
| Cinnamon leaf oil | 0.03-0.1 |
| Dow corning Siliocone fluid | 0.5-2.0 |
| pH 4.0 | |

In a specific non-limiting embodiment, a hand disinfectant soap (referred to as “SynBotanica-3A”) is provided comprising the following concentrations within the ranges set forth in the preceding paragraph:

| Ingredients | % w/w |
|---|---|
| Water | 71.20 |
| Polyox N-60K | 0.15 |
| U-care JR 30M | 0.2 |
| Pluronic F-87 | 2.0 |
| Cocamicopropyl betaine | 6.0 |
| Citric acid | 0.5 |
| Purasil mild | 1.5 |
| SDA-40 B | 10.0 |
| Benzalkanoium chloride | 0.13 |
| Cosmocil | 0.5 |
| Lemon extract | 0.6 |
| Lemon grass oil | 0.2 |
| Phenoxy ethanol | 0.7 |
| Thymol | 0.05 |
| Aloe Juice | 1.0 |
| Glycerin | 1.0 |
| Solubulizer(611624) | 0.5 |
| Plantasol | 2.0 |
| Octanediol | 0.7 |
| Cinnamon leaf oil | 0.07 |
| Dow corning Siliocone fluid 193 | 1.0 |

In certain non-limiting embodiments, a first aid cream (referred to as “SynBotanica First Aid Cream”) is provided comprising the following ranges of ingredients:

| Ingredients | % w/w |
|---|---|
| PHMB | 0.01-0.5 |
| Lemon extract | 0.05-2.0 |
| Cinnamon bark oil | 0.01-1.0 |
| Pomegranite seed oil | 0.1-0.5 |
| Portulaca extract | 0.1-2.0 |
| Aloe gel | 30-70 |
| Natural petrolactum | 10-40 |
| 1,3 propanediol | 1-10 |
| Oat flour | 0.1-5.0 |
| Water | 10-30 |
| Zinc oxide | 0.0-1.0 |
| Germal plus | 0.05-1.0 |

In a specific non-limiting embodiment, a first aid cream (referred to as “SynBotanica First Aid Cream”) is provided comprising the following concentrations within the ranges set forth in the preceding paragraph:

| Ingredients | % w/w |
|---|---|
| PHMB | 0.06 |
| Lemon extract | 0.15 |
| Cinnamon bark oil | 0.1 |
| Pomegranite seed oil | 0.25 |
| Portulaca extract | 0.5 |
| Aloe gel | 40 |
| Natural petrolactum | 20 |
| 1,3 propanediol | 7 |
| Oat flour | 2 |
| Water | 29.64 |
| Zinc oxide | 0 |
| Germal plus | 0.3 |

In any of the formulations above, the percentage of water is intended to bring the total percentage of other ingredients up to 100.

In certain non-limiting embodiments, a body wash (referred to as “SynBotanica body wash-10”) is provided comprising the following ranges of ingredients:

| Ingredients | % w/w |
|---|---|
| Water | 50-90 |
| Guar gum, 2-hydroxyethyl ether | 0.1-1.0 |
| Xanthan gum | 0.1-1.0 |
| Citric/Lactic acid | 0.01-1.0 |
| Cocamidepropyl betaine | 2.0-10.0 |
| Natural Surfactant (Purasil mild) | 0.5-4.0 |
| PHMB | 0.01-0.5 |
| Lemon extract | 0.3-0.6 |
| Cinnamon bark oil | 0.05-0.1 |
| Lemon grass oil | 0.05-0.1 |
| Rosemary oil | 0.05-0.1 |
| Aloe gel | 1.0-10 |
| Octanediol | 0.1-1.0 |
| Glycerine | 1-10 |
| Phenoxy ethanol | 0.0-1.0 |
| Additional Botanical extract | 0.0-1.0 |

In a specific non-limiting embodiment, a body wash (“SynBotanica body wash-10”) is provided comprising the following concentrations within the ranges set forth in the preceding paragraph:

| Ingredients | % w/w |
|---|---|
| Water | 76.3 |
| Guar gum, 2-hydroxyethyl ether | 0.5 |
| Xanthan gum | 0.5 |
| Citric acid | 0.1 |
| Cocamidepropyl betaine | 7.0 |
| Purasil mild | 2.0 |
| PHMB | 0.3 |
| Lemon extract | 0.5 |
| Cinnamon bark oil | 0.1 |
| Lemon grass oil | 0.1 |
| Rosemary oil | 0.1 |
| Aloe gel | 3.0 |
| Octanediol | 0.5 |
| Glycerine | 8.0 |
| Phenoxy ethanol | 0.7 |
| Grape Fruit seed Extract | 0.3 |

In a specific non-limiting embodiment, a hand sanitizer aqueous foam (“Hand sanitizer aqueous foam”) is provided comprising the following ingredients:

| Main ingredients | % w/w |
|---|---|
| Octanediol | 0.8 |
| Lactic acid | 0.2 |
| Alcohol | 10.0 |
| Lemon oil | 0.10 |
| Lemon grass oil | 0.1 |
| Orange oil | 0.1 |
| Thymol | 0.05 |
| Vanilla extract | 0.3 |
| Cosmocil (PHMB) | 1.0 (0.2) |
| Benzalkonium chloride | 0.13 |
| Benzoic acid | 0.2 |
| Phenyl ethanol | 0.3 |
| Other ingredients | % w/w |
| Water | 81.69 |
| Glycerin | 1.0 |
| Hydroxypropyl methyl cellulose | 0.03 |
| stearoxy ether (Sangelose) 90 L | |
| Solubilizer (PEG-40 hydrogenated | 1.0 |
| Castor oil, Trideceth 9, Water) | |
| Plantasol | 1.0 |
| 2-Methyl-1,3-propanediol | 0.75 |
| (Solidol MP-D) | |
| Ethoxy diglycol | 0.5 |
| Isododocane and isononanoate | 0.5 |
| Silicone KF-6043 | 0.25 |

In a specific non-limiting embodiment, an aqueous hand sanitizer lotion (“Aqueous Hand Sanitizer Lotion-1”) is provided comprising the following ingredients:

| Main ingredients | % w/w |
|---|---|
| Octanediol | 0.8 |
| Lactic acid | 0.2 |
| Alcohol | 10.0 |
| Lemon oil | 0.10 |
| Lemon grass oil | 0.1 |
| Orange oil | 0.1 |
| Thymol | 0.05 |
| Vanilla extract | 0.3 |
| Cosmocil (PHMB) | 1.0 (0.2) |
| Benzalkonium chloride | 0.13 |
| Benzoic acid | 0.2 |
| Phenyl ethanol | 0.3 |
| Other ingredients | |
| Water | 74.17 |
| Hydroxypropyl methyl-cellulose | 0.2 |
| stearoxy ether (Sangelose 90 L) | |
| Glycerin | 1.0 |
| Incroquat TMS | 1.5 |
| Polowax NF | 1.0 |
| Stearyl alcohol | 0.8 |
| Zinc oxide | 0.4 |
| Silicone fluid (Dimethicone200/350) | 2.0 |
| Almond oil | 1.5 |
| Lactic acid | 0.1 |
| Aloe juice | 2.0 |
| Bisabolol | 0.05 |
| Zemea | 1.0 |
| Silicone KF 6043 | 1.0 |
| Adjust pH to 4.0-4.5 | |

In a specific non-limiting embodiment, an aqueous hand sanitizer lotion (“Aqueous Hand Sanitizer Lotion-2”) is provided comprising the following ingredients:

| Main ingredients | % w/w |
|---|---|
| Octanediol | 0.8 |
| Lactic acid | 0.2 |
| Alcohol | 10.0 |
| Lemon oil | 0.10 |
| Lemon grass oil | 0.1 |
| Orange oil | 0.1 |
| Thymol | 0.05 |
| Vanilla extract | 0.3 |
| Cosmocil (PHMB) | 1.0 (0.2) |
| Benzalkonium chloride | 0.13 |
| Benzoic acid | 0.2 |
| Phenyl ethanol | 0.3 |
| Other ingredients | |
| Water | 69.77 |
| Oat powder | 1.0 |
| Hydroxypropyl methyl-cellulose | 0.2 |
| stearoxy ether (Sangelose) | |
| Glycerin | 4.0 |
| Incroquat Behenyl TMS | 2.0 |
| Polowax NF | 1.0 |
| Stearyl alcohol | 0.8 |
| Zinc oxide | 0.6 |
| Silicone fluid (Dimethicone200/350) | 2.0 |
| Almond oil | 2.0 |
| Lactic acid | 0.3 |
| Aloe juice | 2.0 |
| Bisabolol | 0.05 |
| Zemea | 1.0 |
| Adjust pH to 4.0-4.5 | |

In a specific non-limiting embodiment, a hand disinfectant soap (“Hand disinfectant Soap-1”) is provided comprising the following ingredients:

| Main ingredients | % w/w |
|---|---|
| Octanediol | 0.5 |
| Citric acid | 0.5 |
| Alcohol | 10.0 |
| Lemon oil | .10 |
| Lemon grass oil | 0.1 |
| Orange oil | 0.1 |
| Thymol | 0.05 |
| Vanilla extract | 0.3 |
| Cosmocil (PHMB) | 1.0 (0.2) |
| Benzalkonium chloride | 0.13 |
| Benzoic acid | 0.2 |
| Other ingredients | |
| Water | 71.97 |
| Polyox N60K | 0.15 |
| Polyquaternium 10 (ucare JR 30) | 0.2 |
| Pluronic surfactant F 87 | 2.0 |
| Cocoamido propyl betaine | 6.0 |
| Purasil Mild | 1.5 |
| Phenoxy ethanol | 0.7 |
| Aloe Juice | 1.0 |
| Glycerin | 1.0 |
| Solubulizer (611624) | 0.5 |
| Plantasol | 1.0 |
| Silicone fluid (Dimethicone200/350) | 1.0 |
| pH 4.0 | |

In a specific non-limiting embodiment, a hand disinfectant soap (“Hand disinfectant Soap-2”) is provided comprising the following ingredients:

| Main ingredients | % w/w |
|---|---|
| Octanediol | 0.5 |
| Citric acid | 0.5 |
| Alcohol | 10.0 |
| Lemon oil | .10 |
| Lemon grass oil | 0.1 |
| Orange oil | 0.1 |
| Thymol | 0.05 |
| Vanilla extract | 0.3 |
| Cosmocil (PHMB) | 1.0 (0.2) |
| Benzalkonium chloride | 0.13 |
| Benzoic acid | 0.2 |
| Other ingredients | |
| Water | 69.32 |
| Polyox N60K | 0.2 |
| Polyquatemium 10 (Ucare JR 30) | 0.3 |
| Pluronic surfactant F 87 | 1.0 |
| Cocoamido propyl betaine | 6.0 |
| Mackam BC 39 | 3.0 |
| Purasil Mild | 2.0 |
| Phenoxy ethanol | 0.7 |
| Aloe Juice | 1.0 |
| Glycerin | 2.0 |
| Solubulizer (611624) | 0.5 |
| Plantasol | 1.0 |
| pH 4.0 | |

In a specific non-limiting embodiment, a hand disinfectant soap (“Hand disinfectant Soap”) is provided comprising the following range of ingredients:

| Ingredients | % w/w |
|---|---|
| Water | 55-70 |
| Polyquaternium 10 | 0.1-0.7 |
| Soft CAT SL 100 | 0.1-0.7 |
| Pluronic surfactant F 87 | 1.0-2.0 |
| Cocoamido propyl betaine | 7-12.0 |
| Purasil Mild | 0.5-3.0 |
| Citric acid | 0.1-0.2 |
| Benzalkanoium chloride | 0.01-0.12 |
| CHG (20%) | 0.25-1.0 |
| Alcohol (SDA40B) | 0-15.0 |
| Lemon grass oil | 0.1-0.5 |
| Orange oil | 0.1-0.2 |
| Thymol | 0.03-0.1 |
| Vanilla oil | 0.1-0.3 |
| Fennel oil | 0.1-0.3 |
| Basil oil | 0.03-0.1 |
| Phenoxy ethanol | 0.5-1.0 |
| Grapefruit seed extract | 0.1-0.6 |
| Aloeveragel/Juice | 2.0-10 |
| Glycerin | 1.0-5.0 |
| Octanediol | 0.5-1.0 |
| Adjust pH to 4.0-4.2 | |

In a specific non-limiting embodiment, a hand disinfectant soap (“Hand disinfectant Soap”) is provided comprising the following ingredients:

| Ingredients | % w/w |
|---|---|
| Water | 69.23 |
| Polyquaternium 10 | 0.5 |
| Soft CAT SL 100 | 0.5 |
| Pluronic surfactant F 87 | 1.0 |
| Cocoamido propyl betaine | 10.0 |
| Purasil Mild | 1.0 |
| Citric acid | 0.2 |
| Benzalkanoium chloride | 0.12 |
| CHG (20%) | 1.0 |
| Alcohol (SDA40B) | 10.0 |
| Lemon grass oil | 0.2 |
| Orange oil | 0.1 |
| Thymol | 0.1 |
| Vanilla oil | 0.1 |
| Fennel oil | 0.1 |
| Basil oil | 0.05 |
| Phenoxy ethanol | 0.7 |
| Grapefruit seed extract | 0.3 |
| Aloe veragel/Juice | 2.0 |
| Glycerin | 2.0 |
| Octanediol | 0.8 |
| Adjust pH to 4.0-4.2 | |

In a specific non-limiting embodiment, a cow teat disinfectant solution is provided comprising the following ingredients:

| Main ingredients | % w/w |
|---|---|
| PHMB | 0.3 |
| Vanilla extract | 0.3 |
| Octanediol | 0.7 |
| Rosemary oil | 0.1 |
| Other ingredients | |
| Aloe juice | 2.0 |
| Propylene glycol | 5.0 |
| Glycerine | 5.0 |
| Water | 86.6 |

In a specific non-limiting embodiment, a hand disinfectant soap (“HAND DISINFECTANT SOAP-SBW-1”) is provided comprising the following ingredients:

| Main ingredients | % w/w |
|---|---|
| Lemon oil | 0.1 |
| Lemon grass oil | 0.1 |
| Orange oil | 0.1 |
| Thymol | 0.05 |
| Vanilla extract | 0.3 |
| PAPB | 0.2 |
| Benzalkonium chloride | 0.13 |
| Other ingredients | |
| Water | 70.12 |
| Polyox N60 K | 0.2 |
| Polyquaternium 10 (U-care JR 30) | 0.3 |
| Pluronic surfactant F 87 | 1.0 |
| Cocamido propyl betaine | 6.0 |
| Puracil mild | 2.0 |
| Phenoxy ethanol | 0.7 |
| Aloe juice | 1.0 |
| Glycerin | 2.0 |
| Solubilizer (611674) | 0.5 |
| Mackam BC-39 | 3.0 |
| Plantasol | 1.0 |
| Ocatnediol | 0.5 |
| Citric acid | 0.5 |
| Alcohol | 10.0 |
| Benzoic acid | 0.2 |

In a specific non-limiting embodiment, an Aqueous Hand Sanitizer Lotion (“SBW lotion-2”) is provided comprising the following ingredients:

| Main ingredients | % w/w |
|---|---|
| Lemon oil | 0.1 |
| Lemon grass oil | 0.1 |
| Orange oil | 0.1 |
| Thymol | 0.05 |
| Vanilla extract | 0.3 |
| PAPB | 0.2 |
| Benzalkonium chloride | 0.13 |
| Other ingredients | |
| Water | 70.77 |
| Sangelose 90 L | 0.2 |
| Glycerin | 4.0 |
| Incroquat Behenyl TMS | 2.0 |
| Polowax NF | 1.0 |
| Stearyl alcohol | 0.8 |
| Oat powder | 1.0 |
| Zinc oxide | 0.6 |
| Silicone fluid (200/300) | 2.0 |
| Almond oil | 2.0 |
| Lactic acid | 0.3 |
| Aloe juice | 2.0 |
| Zemea | 1.0 |
| SDA 40B | 10.0 |
| Benzoic acid | 0.2 |
| Phenyl ethanol | 0.3 |
| Octanediol | 0.8 |
| Bisabolol | 0.05 |

In a specific non-limiting embodiment, an anti-inflammatory, anti-irritant topical gel (“SynBotanica Anti-inflammatory, anti-irritant topical gel SB AI-3”) is provided comprising the following ingredients:

| Ingredients | % w/w |
|---|---|
| Main ingredients | |
| PHMB | 0.6 |
| Lemon/citrus extract | 0.15 |
| Cinnamon oil | 0.05 |
| Rosemary oil | 0.1 |
| Octanediol | 0.8 |
| Oat Powder | 2.0 |
| Tetrahydrocurcumanoid | 0.1 |
| Other ingredients | |
| Water | 15.45 |
| Lactic acid | 0.1 |
| Natural Petrolactum | 40 |
| Aloe Gel | 30.0 |
| Pomegranate oil | 0.3 |
| Portulaca extract | 0.75 |
| Lemon grass oil | 0.1 |
| Tocopherol | 0.2 |
| Camphor | 0.3 |
| Dracocorin( Natural emulsifier) | 2.0 |

In certain non-limiting embodiments, a cow teat disinfectant (CT) is provided comprising the following range of ingredients:

| Ingredients | % w/w (5X) | % w/w (1X) |
|---|---|---|
| Water | 14.0-18.0 | 70.0-90.0 |
| Glycerine | 15.0-30.0 | 3.0-6.0 |
| Propylene glycol | 10.0-30.0 | 2.0-6.0 |
| Octanediol | 2.0-4.0 | 0.4-0.8 |
| Solubilizer (660352) | 5.0-25.0 | 1.0-5.0 |
| Chlorhexidine | 0.3-5.0 | 0.06-1.0 |
| Phenoxy ethanol | 2.5-5.0 | 0.5-1.0 |
| Grape fruit seed extract | 1.5-5.0 | 0.3-1.0 |
| Aloe gel 1X/Aloe leaf juice | 10.0-25.0 | 2.0-5.0 |
| PlantaSol | 1.25-15.0 | 0.25-3.0 |
| Thymol | 0.25-1.0 | 0.05-0.2 |
| Orange oil | 0.25-1.0 | 0.05-0.2 |
| Lemongrass oil | 0.25-1.0 | 0.05-0.2 |
| Citric acid (15%) | 0.25-2.0 | 0.05-0.4 |

In certain non-limiting embodiments, a cow teat disinfectant (“CT-43”) is provided comprising the following:

| Ingredients | % w/w |
|---|---|
| Water | 81.8 |
| Glycerine | 5.0 |
| Propylene glycol | 5.0 |
| Octanediol | 0.5 |
| Solubilizer (660352) | 2.0 |
| Chlorhexidine | 0.5 |
| Phenoxy ethanol | 0.7 |
| Grape fruit seed extract | 0.5 |
| Aloe gel 1X | 3.0 |
| PlantaSol | 0.5 |
| Thymol | 0.1 |
| Orange oil | 0.1 |
| Lemongrass oil | 0.1 |
| Citric acid (15%) | 0.2 |
| pH 4.1-4.2 | |

In certain non-limiting embodiments, a cow teat disinfectant (“CT-49”) is provided comprising the following:

| Ingredients | % w/w |
|---|---|
| Water | 82.05 |
| Glycerine | 5.0 |
| Propylene glycol | 5.0 |
| Octanediol | 0.5 |
| Solubilizer (660352) | 2.0 |
| Chlorhexidine | 0.25 |
| Phenoxy ethanol | 0.7 |
| Grape fruit seed extract | 0.5 |
| Aloe gel 1X | 3.0 |
| PlantaSol | 0.5 |
| Thymol | 0.1 |
| Orange oil | 0.1 |
| Lemongrass oil | 0.1 |
| Citric acid (15%) | 0.2 |
| pH 4.1-4.2 | |

In certain non-limiting embodiments, a cow teat disinfectant (“CT-51”) is provided comprising the following:

| Ingredients | % w/w |
|---|---|
| Water | 82.18 |
| Glycerine | 5.0 |
| Propylene glycol | 5.0 |
| Octanediol | 0.5 |
| Solubilizer (660352) | 2.0 |
| Chlorhexidine | 0.125 |
| Phenoxy ethanol | 0.7 |
| Grape fruit seed extract | 0.5 |
| Aloe gel 1X | 3.0 |
| PlantaSol | 0.5 |
| Thymol | 0.1 |
| Orange oil | 0.1 |
| Lemongrass oil | 0.1 |
| Citric acid (15%) | 0.2 |
| pH 4.1-4.2 | |

In certain non-limiting embodiments, a mastitis cream is provided comprising the following:

| Ingredients | % w/w |
|---|---|
| Isopropyl myristate | 1.2 |
| Sorbitan oleate | 0.5 |
| Stearyl alcohol | 3.5 |
| Polyoxyl 40 stearate (Myrj 52) | 1.3 |
| Arlacel | 0.6 |
| Water | 77.74 |
| Germal1+ | 0.06 |
| Propylene glycol | 1.0 |
| White petrolatum | 1.0 |
| Chlorhexidine gluconate | 0.2 |
| Almond oil | 2.0 |
| Jojoba oil | 2.0 |
| Oat flour | 2.0 |
| Zinc oxide | 0.3 |
| Aloe Gel 1x | 5.0 |
| Thymol | 0.1 |
| Fennel oil | 0.1 |
| Vanilla oil | 0.1 |
| Basil oil | 0.1 |
| Grapefruit seed extract | 0.3 |
| Calendula oil | 0.3 |
| Pomogranate oil | 0.3 |
| Rosemary oil | 0.1 |
| Curcumin | 0.2 |

### 4.4 Surface Disinfectants

Disclosed herein are compositions that may be comprised in disinfectant compositions that may be used to topically to reduce the number and/or growth of microbes on inanimate surfaces, for use in disinfecting, for example and not by way of limitation, hard surfaces such as floors, countertops, sinks, toilets, shower stalls, tables, furniture, car interiors, baby furniture, etc. as well as soft surfaces but non-absorbent surfaces such as leather or vinyl.

The disinfectant composition comprises the recited active agents and a suitable solvent. Non-limiting examples of suitable solvents include water, ethanol, glycerin, octoxyglycerin, and mixtures thereof.

Disclosed herein is a method of reducing the number and/or growth of microbes on an inanimate surface comprising applying to the surface a disinfectant composition as set forth herein. Microbes, the numbers and/or growth of which are reduced, include but are not limited to one or more of Staphylococcus aureus (including methicillin resistant Staphylococcus aureus), Staphylococcus epidermidis, Escherichia cob, Pseudomonas aeruginosa, Candida albicans, Aspergillus niger and Listeria monocytogenes.

In certain non-limiting embodiments, a hard surface disinfecting agent is provided comprising (a) one or more plant/fruit extract selected from the group consisting of citrus extract, cedarwood extract, lemon extract or grapefruit seed extract, vanilla extract or a combination thereof (e.g. at a concentration between about 0.1 and 0.6% w/w or at a concentration between about 0.0 and 2.0% w/w); (b) one or more synthetic antimicrobial which may be a quaternary ammonium compound such as benzalkonium chloride, benzethonium chloride, or cetyl pyrinidium chloride, a biguanide such as chlorhexidine free base or chlorhexidine salt (e.g. chlorhexidine gluconate or chlorhexidine acetate) or polyhexamethylene biguanide, or a combination thereof (e.g. at a concentration between about 0.01 and 1.0% w/w); (c) one or more essential oil or constituent thereof which may be thymol, lemongrass oil, pine oil, cedarwood oil or a combination thereof (e.g. at a concentration between about 0.01 and 5.0% w/w); (d) one or more plant based organic acid which may be lactic acid, citric acid, benzoic acid, or a combination thereof (e.g. at a concentration between about 0.1 and 5.0% w/w or at a concentration between about 5.0 and 20.0% w/w); and (e) one or more eco friendly surfactant, for example an alkyl polyglucoside, capryl glucoside, a cationic surfactant blend, or a non-ionic surfactant blend.

In certain non-limiting embodiments, a surface disinfectant is provided comprising the following ranges of ingredients:

| Ingredients | % w/w (10X) | % w/w (1X) |
|---|---|---|
| Thymol | 0.1-1.0 | 0.01-0.1 |
| Pine oil | 0.2-4.0 | 0.02-0.4 |
| Surfactant (Diversaclean SRS) | 5.0-15.0 | 0.5-1.5 |
| Surfactant (Glucopon 415N) | 2.0-10.0 | 0.2-1.0 |
| Citric acid | 5.0-20.0 | 0.5-2.0 |
| Botanical extract | 0.0-2.0 | 0.0-0.2 |
| Vantocil | 0.0-1.0 | 0.0-0.1 |
| Alkyl Dimethyl Benzyl ammonium chloride | 0.0-0.2 | 0.0-0.02 |
| Urea hydrogen peroxide | 0.0-2.0 | 0.0-0.2 |
| Sodium Hypochlorite | 0.0-0.8 | 0.0-0.08 |
| SDA 3C | 15.0-50.0 | 1.5-5.0 |
| Lemon grass oil | 0.1-1.0 | 0.01-0.1 |
| Water | 30-70 | 97.0-90.0 |

In certain non-limiting embodiments, a surface disinfectant is provided comprising the following ranges of ingredients:

| Ingredients | % w/w |
|---|---|
| Thymol | 0.1-1.0 |
| Pine oil | 0.2-4.0 |
| Surfactant (Diversaclean SRS) | 5.0-15 |
| Surfactant (Glucopon 415N) | 2-10.0 |
| Citric acid | 5-20 |
| Lemon extract | 0.02-2.0 |
| Vantocil | 0.01-1.0 |
| SDA 3C | 15-50 |
| Lemon grass oil | 0.1-1.0 |
| Water | 30-70 |

The above solution may be diluted with water, for example in a ratio between about 1:5 and 1:10, prior to use.

In a specific non-limiting embodiment, a surface disinfectant is provided comprising the following concentrations within the ranges set forth in the preceding paragraph:

| Ingredients | % w/w |
|---|---|
| Thymol | 0.5 |
| Pine oil | 2.0 |
| Diversaclean SRS | 8.0 |
| Glucopon 415N | 5.0 |
| Citric acid | 10.0 |
| Lemon extract | 1.0 |
| Vantocil | 1.0 |
| Lemon grass oil | 0.2 |
| SDA 3C | 25.0 |
| Water | 47.3 |

The above solution may be diluted with water, for example in a ratio between about 1:5 and 1:10, prior to use.

In a specific non-limiting embodiment, a Surface Disinfectant General formula 1 is provided comprising the following ranges of ingredients:

| Ingredients | % w/w |
|---|---|
| Pine oil | 0.2-4.0 |
| Cedarwood oil | 1-10 |
| Surfactant(Diversaclean SRS ) | 5.0-15 |
| Surfactant(Glucopon 415N) | 0-10.0 |
| Citric acid | 5-20 |
| Lemon extract | 0.02-2.0 |
| Vantocil* | 0.01-1.0 |
| SDA 3C | 15-50 |
| Lemon grass oil | 0.1-1.0 |
| Water | 30-70 |

*Vantocil is 20% w/w PHMB

The above solution may be diluted with water, for example, in a ratio between about 1:5 and 1:10, prior to use.

In a specific non-limiting embodiment, a Specific formulation 1A is provided comprising the following concentrations within the ranges set forth in the preceding paragraph:

| Ingredients | % w/w |
|---|---|
| Pine oil | 1.0 |
| Cedarwood oil | 1.0 |
| Surfactant (Diversaclean SRS) | 10.0 |
| Surfactant (Glucopon 415N) | — |
| Citric acid | 10 |
| Lemon extract | 0.5 |
| Vantocil* | 0.2 |
| SDA 3C | 25 |
| Lemon grass oil | 0.1 |
| Water | 52.2 |

*Vantocil is 20% w/w PHMB

The above solution may be diluted with water, for example, in a ratio between about 1:5 and 1:10, prior to use.

### 5. EXAMPLE 1

### 5.1 Formulations Tested

The antibacterial activity of various test formulations were tested. The test formulations were prepared by adding active agents to the following base formulation, in the concentrations listed in Table 1. Active ingredients were lemongrass oil (“LG”), cinnamon bark oil (“CN”), thymol (“T”), benzalkanoium chloride (“BAC”), polyhexamethylene biguanide (“PHMB”) and octanediol (“O”). Where needed water was added to bring the concentration to 100%.

Base formulation

| Ingredients | % (w/w) |
|---|---|
| Water | 81.0 |
| Sanglose | 0.025 |
| Solubulizer (Peg-40 hydrogented | 2.0 |
| Castor oil, Trideceth 9, water) | |
| Plantasol | 3.0 |

### 5.2 Testing Method

Antibacterial activity against methicillin-resistant Staphylococcus aureus (“MRSA”) was tested using a 15 second exposure time (hereafter, “MRSA Rapid-Kill Assay”). Briefly, a tube-dilution method was used to evaluate samples as per the US FDA-Tentative Final Monograph (“TFM”) method. 0.9 ml of test sample was added to 0.1 ml of a bacterial culture containing 1×10⁷ colony forming units per ml [cfu/ml] and vortexed for 15 seconds. 9 ml of Drug Neutralizing fluid (DNF) was added to neutralize activity, diluted serially and 0.5 ml plated on TSA plates. Plates were incubated for 18-24 hrs at 37° C. Base formulation without additives was used as a control in place of test sample and processed similarly. The antibacterial activity was determined by calculating the difference in log₁₀ cfu/ml of test samples relative to control [PBS]. The results are shown in Table 1.

### 5.3 Results

TABLE 1

| | Conc. | Log |
|---|---|---|
| | Active | reduction |
| Compounds | Agent | from |
| growth | (% w/w) | control |
| LG + CN | 0.1 + 0.1 | 0.86 |
| LG + CN + T | 0.1 + 0.1 + 0.10 | 0.88 |
| BAC | 0.13 | 0.05 |
| PHMB | 0.012 | 0.4 |
| LG + CN + BAC | 0.1 + 0.1 + 0.13 | 1.03 |
| LG + CN + T + BAC | 0.1 + 0.1 + 0.1 + 0.13 | 1.47 |
| LG + CN + PHMB | 0.1 + 0.1 + 0.012 | 1.1 |
| LG + CN + T + PHMB | 0.1 + 0.1 + 0.1 + 0.012 | 1.3 |
| PHMB | 0.06 | 3.1 |
| CN | 0.2 | 0.98 |
| LG | 0.2 | 0.92 |
| CN + PHMB | 0.2 + 0.06 | 5.5 |
| LG + PHMB | 0.2 + 0.06 | 5.0 |
| O | 0.7 | 1.7 |
| LG + CN + O | 0.1 + 0.1 + 0.7 | 2.13 |
| BAC + O | 0.13 + 0.7 | 1.72 |
| LG + CN + BAC + O | 0.1 + 0.1 + 0.13 + 0.7 | 2.94 |
| LG + CN + T + BAC + O | 0.1 + 0.1 + 0.1 + 0.13 + 0.07 | 3.73 |

Control growth ranged from 5×10⁵-1×10⁶. It was observed that BAC enhanced the activity of essential oil mixtures containing thymol; BAC+ octanediol further enhanced the activity of LG+CN+ T; and PHMB at 0.06% showed synergistic activity with essential oils.

### 6. EXAMPLE 2

### 6.1 Formulations Tested

The antibacterial activity of various test formulations were tested. The test formulations were prepared by adding active agents to the following base formulation, in the concentrations listed in Table 2. Active ingredients were benzalkanoium chloride (“BAC”), polyhexamethylene biguanide (“PHMB”), lemon extract (“LE”), wasabi extract (“W”), and aspen bark extract (“A”). Where necessary water was added to bring the total concentration to 100%.

Base formulation

| Ingredients | % (w/w) |
|---|---|
| Water | 81.0 |
| Sanglose | 0.025 |
| Solubulizer (Peg-40 hydrogented | 2.0 |
| Castor oil, Trideceth 9, water) | |
| Plantasol | 3.0 |

### 6.2 Testing Method

Antibacterial activity against methicillin-resistant Staphylococcus aureus (“MRSA”) was tested using a 15 second exposure time (hereafter, “MRSA Rapid-Kill Assay”). Briefly, a tube-dilution method was used to evaluate samples as per the US FDA-Tentative Final Monograph (“TFM”) method. 0.9 ml of test sample was added to 0.1 ml of a bacterial culture containing 1×10⁷ colony forming units per ml [cfu/ml] and vortexed for 15 seconds. 9 ml of Drug Neutralizing fluid (DNF) was added to neutralize activity, diluted serially and 0.5 ml plated on TSA plates. Plates were incubated for 18-24 hrs at 37° C. Base formulation without additives was used as a control in place of test sample and processed similarly. The antibacterial activity was determined by calculating the difference in log 10 cfu/ml of test samples relative to control [PBS]. The results are shown in Table 2.

### 6.3 Results

TABLE 2

| | Conc. | Log |
|---|---|---|
| | Active | reduction |
| Compounds | Agent | from |
| growth | (% w/w) | control |
| BAC | 0.13 | 0.05 |
| LE | 0.3 | 0.5 |
| LE + BAC | 0.3 + 0.13 | 0.93 |
| PHMB | 0.012 | 0.4 |
| LE + PHMB | 0.3 + 0.012 | 1.0 |
| W | 0.3 | 0.5 |
| W + BAC | 0.3 + 0.13 | 0.5 |
| A | 0.3 | 0.55 |
| A + BAC | 0.3 + 0.13 | 0.60 |
| LE + W + A + BAC | 0.3 + 0.3 + 0.3 + 0.13 | 1.15 |

Control growth ranged from 5×10⁵-1×10⁶. It was observed that lemon extract showed enhanced activity with BAC

### 7. EXAMPLE 3

### 7.1 Formulations Tested

The antibacterial activity of various test formulations was tested. The test formulations were prepared by adding active agents (see Table 3) to the same base formulation used in the preceding example section. Active ingredients were citrus extract (lemon and grapefruit seed extracts), cinnamon bark oil (“CN”), thymol (“T”), clove bud oil (“CL”), pentanediol (“P”) and polyhexamethylene biguanide (“PHMB”). Where necessary water was added to bring the total concentration to 100%.

Base formulation

| Ingredients | % (w/w) |
|---|---|
| Water | 81.0 |
| Sanglose | 0.025 |
| Solubulizer (Peg-40 hydrogented | 2.0 |
| Castor oil, Trideceth 9, water) | |
| Plantasol | 3.0 |

### 7.2 Testing Method

For oral care products, PHMB is preferred because BAC can produce mucosal irritation. PHMB is effective against gram negative bacteria. Therefore P. aeruginosa which is a gram negative organism was used in these tests. An agar-plate method was used/0.5 ml of the test sample was spread on TSA plates and for control PBS was used in the plate. These plates were incubated for 1 hr in the incubator at temperature 37±1° C. After 1 hr, the plates were taken out of the incubator and inoculated with 0.5 ml of 1×10³ cfu/ml culture of P. aeruginosa. The plates were incubated for 18-24 hrs at 37±1° C. The antibacterial activity was determined by calculating the difference in log 10 colony-forming units (cfu) between control and test samples (i.e., log 10 reduction). The results are shown in Table 3 w.

### 7.3 Results

TABLE 3

| | Conc. | Log |
|---|---|---|
| | Active | reduction |
| Compounds | Agent | from |
| growth | (% w/w) | control |
| PHMB + P | 0.1 + 1.0 | 0.5 |
| CN + CL + T + | 0.05 + 0.05 + 0.05 | 0.6 |
| Citrus extract | | |
| CN + CL + T + | 0.05 + 0.05 + 0.05 + 1.0 + 1.0 | 2.1 |
| Citrus extract | | |
| +PHMB + P | | |

Control growth ranged from 5×10⁵-1×10⁶. It was observed that PHMB and pentanediol enhanced the activity of low concentrations of essential oils and lemon extract.

### 8. EXAMPLE 4

### 8.1 Formulations Tested

The antibacterial activity of various test formulations were tested. The test formulations were prepared by adding active agents to the same base formulation used above, in the concentrations listed in Table 4. Active ingredients were lemongrass oil (“LG”), cinnamon bark oil (“CN”), thymol (“T”), benzalkonium chloride (“BAC”), polyhexamethylene biguanide (“PHMB”), lemon extract (“LE”), and octanediol (“0”). Where necessary water was added to bring the total concentration to 100%.

Base formulation

| Ingredients | % (w/w) |
|---|---|
| Water | 81.0 |
| Sanglose | 0.025 |
| Solubulizer (Peg-40 hydrogented | 2.0 |
| Castor oil, Trideceth 9, water) | |
| Plantasol | 3.0 |

### 8.2 Testing Method

Antibacterial activity against methicillin-resistant Staphylococcus aureus (“MRSA”) was tested using a 15 second exposure time (hereafter, “MRSA Rapid-Kill Assay”). Briefly, a tube-dilution method was used to evaluate samples as per the US FDA-Tentative Final Monograph (“TFM”) method. 0.9 ml of test sample was added to 0.1 ml of a bacterial culture containing 1×10⁷ colony forming units per ml [cfu/ml] and vortexed for 15 seconds. 9 ml of Drug Neutralizing fluid (DNF) was added to neutralize activity, diluted serially and 0.5 ml plated on TSA plates. Plates were incubated for 18-24 hrs at 37° C. Base formulation without additives was used as a control in place of test sample and processed similarly. The antibacterial activity was determined by calculating the difference in log 10 cfu/ml of test samples relative to control [PBS]. The results are shown in Table 4.

### 8.3 Results

TABLE 4

| Compounds | Conc. | Log |
|---|---|---|
| growth | Active | reduction |
| In base | Agent | from |
| formulation | (% w/w) | control |
| LG + CN + T | 0.1 + 0.1 + 0.07 | 0.88 |
| LE + BAC + O | 0.3 + 0.13 + 0.7 | 2.27 |
| LE + BAC + O + | 0.3 + 0.13 + 0.7 + | 3.31 |
| LG + CN + T | 0.1 + 0.1 + 0.07 | |
| LE + PHMB + O | 0.3 + 0.06 + 0.7 | 2.8 |
| LE + PHMB + O + | 0.3 + 0.06 + 0.7 | 5.8 |
| LG + CN + T | 0.1 + 0.1 + 0.07 | |

Control growth ranged from 5×10⁵-1×10⁶. It was observed that combinations of lemon extract, PHMB and octanediol showed synergistic activity with essential oils. The above data indicate that (1) of the extracts tested, only lemon extract appears to show synergistic activity with BAC; (2) BAC enhances the activity of essential oils (CN+LG+T); and (3) lemon extract+PHMB+alkanediols appear to exhibit synergism with essential oils.

### 9. EXAMPLE 5

### 9.1 Formulations Tested

The antibacterial activity of various test formulations were tested. The test formulations were prepared by adding active agents to the following base formulation, in the concentrations listed in Table 5A-F. Active ingredients were lemongrass oil (“LG”), cinnamon bark oil (“CN”), thymol (“T”), benzalkanoium chloride (“BAC”), polyhexamethylene biguanide (“PHMB”), octanediol (“0”), pentanediol (“P”), wasabi extract (“WE”), aspen bark extract (“AE”), lemon extract (“LE”), vanilla extract (“VE”), cetyl pyridinium chloride (“CPC”), and rosemary (“R”). Where necessary water was added to bring the total concentration to 100%.

Base formulation

| Ingredients | % (w/w) |
|---|---|
| Water | 81.0 |
| Sanglose | 0.025 |
| Solubulizer (Peg-40 hydrogented | 2.0 |
| Castor oil, Trideceth 9, water) | |
| Plantasol | 3.0 |

### 9.2 Testing Method

Antibacterial activity against methicillin-resistant Staphylococcus aureus (“MRSA”) was tested using a 15 second exposure time (hereafter, “MRSA Rapid-Kill Assay”). Briefly, a tube-dilution method was used to evaluate samples as per the US FDA-Tentative Final Monograph (“TFM”) method. 0.9 ml of test sample was added to 0.1 ml of a bacterial culture containing 1×10⁷ colony forming units per ml [cfu/ml] and vortexed for 15 seconds. 9 ml of Drug Neutralizing fluid (DNF) was added to neutralize activity, diluted serially and 0.5 ml plated on TSA plates. Plates were incubated for 18-24 hrs at 37° C. Base formulation without additives was used as a control in place of test sample and processed similarly. The antibacterial activity was determined by calculating the difference in log₁₀ cfu/ml of test samples relative to control [PBS]. The results are shown in Tables 5A-F.

### 9.3 Results

TABLE 5A
Efficacy of BAC with plant extracts

| | Conc. | Log |
|---|---|---|
| | Active | reduction |
| Compounds | Agent | from |
| growth | (% w/w) | control |
| BAC | 0.6 | 0.5 |
| WE | 0.6 | 0.05 |
| AE | 0.6 | 0.05 |
| LE | 0.6 | 0.05 |
| VE | 0.6 | 0.05 |
| LE + BAC | 0.6 + 0.13 | 1.29 |
| WE + BAC | 0.6 + 0.13 | 0.73 |
| VE + BAC | 0.6 + 0.13 | 1.37 |
| AE + BAC | 0.6 + 0.13 | 0.80 |

Control growth range from 5 × 10⁵-1 × 10⁶

TABLE 5B
Efficacy of PHMB with plant extracts

| | Conc. | Log |
|---|---|---|
| | Active | reduction |
| Compounds | Agent | from |
| growth | (% w/w) | control |
| PHMB | 0.06 | 4.0 |
| WE | 0.6 | 0.05 |
| AE | 0.6 | 0.05 |
| LE | 0.6 | 0.05 |
| VE | 0.6 | 0.05 |
| WE + PHMB | 0.6 + 0.3 | 3.25 |
| AE + PHMB | 0.6 + 0.3 | 3.2 |
| LE + PHMB | 0.6 + 0.3 | 6.52 |
| VE + PHMB | 0.6 + 0.3 | 6.52 |

Control growth range from 5 × 10⁵-1 × 10⁶

TABLE 5C
Efficacy of BAC and PHMB with plant extracts

| | Conc. | Log |
|---|---|---|
| | Active | reduction |
| Compounds | Agent | from |
| growth | (% w/w) | control |
| BAC + PHMB 0 | 0.13 + 0.06 | 3.2 |
| LE | 0.6 | 0.1 |
| VE | 0.6 | 0.1 |
| LE + PHMB + BAC | 0.6 + 0.06 + 0.13 | 6.52 |
| VE + PHMB + BAC | 0.6 + 0.06 + 0.13 | 6.52 |
| LE + VE + PHMB + BAC | 0.15 + 0.15 + 0.06 + 0.13 | 5.32 |

Control growth range from 5 × 10⁵-1 × 10⁶

TABLE 5D
Efficacy of BAC, PHMB and O with plant extracts

| | Conc. | Log |
|---|---|---|
| | Active | reduction |
| Compounds | Agent | from |
| growth | (% w/w) | control |
| LE + VE | 0.3 + 0.3 | 0.1 |
| LE + VE | 0.15 + 0.15 | 0.085 |
| O + PHMB + BAC | 0.7 + 0.06 + 0.13 | 2.33 |
| LE + O + PHMB + BAC | 0.6 + 0.06 + 0.7 + 0.13 | 6.85 |
| VE + O + PHMB + BAC | 0.6 + 0.06 + 0.7 + 0.13 | 6.85 |
| LE + VE + O + PHMB + BAC | 0.3 + 0.3 + 0.06 + + 0.7 + 0.13 | 6.85 |
| LE + VE + O + PHMB + BAC | 0.15 + 0.15 + 0.06 + 0.7 + 0.13 | 5.40 |

TABLE 5E
Efficacy of PHMB, P and CPC with plant extracts

| | Conc. | Log |
|---|---|---|
| | Active | reduction |
| Compounds | Agent | from |
| growth | (% w/w) | control |
| P + PHMB | 0.7 + 0.06 | 3.2 |
| LE + VE | 0.3 + 0.3 | 0.1 |
| P + PHMB + CPC | 0.7 + 0.06 + 0.13 | 3.63 |
| LE + VE + P + PHMB + CPC | 0.3 + 0.3 + 0.7 + 0.13 + 0.06 | 6.85 |

TABLE 5F
Efficacy of Essential Oils, PHMB, BAC and
Alkanediols with plant extracts

| | Conc. | Log |
|---|---|---|
| | Active | reduction |
| Compounds | Agent | from |
| growth | (% w/w} | control |
| T + R | 0.07 + 0.1 | 0.07 |
| O + PHMB + BAC | 0.7 + 0.06 + 0.13 | 2.33 |
| T + R + O + PHMB + BAC | 0.07 + 0.2 + 0.7 + 0.06 + 0.13 | 2.96 |
| LG + CN | 0.1 + 0.1 | 0.86 |
| LG + CN + T + R + O + | 0.1 + 0.1 + 0.07 + 0.1 + 0.7 + 0.06 | 5.63 |
| PHMB + BAC | | |

The above results indicate that of the plant extracts tested, vanilla and citrus extracts, which contain high concentrations of polyphenols (30-50% and above), exhibit significant synergism with low concentrations of biguanide (PHMB) (0.06%) and relatively lower synergism with 0.13% w/w of BAC. These extracts, along with BAC+PHMB with and without alkanediols, were observed to effect 100% kill of pathogens. Other extracts also enhanced the activity but not as significantly as LE and VE when used along with BAC+PHMB+O. Synergistic activity of these extracts at the use concentrations indicated above (0.3-0.6% w/w) were not observed to exhibit detectable odor as tested. Of note, the essential oils and thymol as used above did produce detectable odor.

In the above tests, essential oils at 0.1-0.3% did not show synergistic activity with BAC+PHMB with and without alkanediols. Combinations of low concentrations of essential oils and lemon or vanilla extract showed synergistic activity with PHMB+O against MRSA.

### 10. EXAMPLE 6

### 10.1 Formulations Tested

Botanical oral rinse “OCP-G1*” was prepared initially as two phases, A and B, which were then mixed:

| Ingredients | (% w/w) |
|---|---|
| Phase A | |
| Water | 85.59 |
| Xylitol | 0.25 |
| Sorbitol | 0.5 |
| Pluronic F087 | 0.1 |
| Lactic acid | 0.1 |
| Zinc salicylate | 0.1 |
| Glycerin | 7.0 |
| Propylene glycol | 3.0 |
| Aloe leaf juice | 1.0 |
| Phase B | |
| Vanilla extract | 0.6 |
| PHMB | 0.12 |
| Cinnamon bark oil | 0.05 |
| Lemon grass oil | 0.01 |
| Clove bud oil | 0.03 |
| Peppermint | 0.05 |
| Hydrolite 5 | 0.5 |
| Polysorbate 80 | 1.0 |

Botanical oral rinse “OCP-G2*” was prepared initially as two phases, A and B, which were then mixed:

| Ingredients | (% w/w) |
|---|---|
| Phase A | |
| Water | 85.59 |
| Xylitol | 0.25 |
| Sorbitol | 0.5 |
| Pluronic F087 | 0.1 |
| Lactic acid | 0.1 |
| Zinc salicylate | 0.1 |
| Glycerin | 7.0 |
| Propylene glycol | 3.0 |
| Aloe leaf juice | 1.0 |
| Phase B | |
| Lemon extract | 0.6 |
| PHMB | 0.12 |
| Cinnamon bark oil | 0.05 |
| Lemon grass oil | 0.01 |
| Clove bud oil | 0.03 |
| Peppermint | 0.05 |
| Hydrolite 5 | 0.5 |
| Polysorbate 80 | 1.0 |

Botanical oral rinse “OCP-AIF*” was prepared initially as two phases, A and B, which were then mixed:

| Ingredients | (% w/w) |
|---|---|
| Phase A | |
| Water | 75.54 |
| Xylitol | 0.25 |
| Sorbitol | 0.5 |
| Pluronic F087 | 0.1 |
| Lactic acid | 0.1 |
| Zinc salicylate | 0.1 |
| Glycerin | 7.0 |
| Propylene glycol | 3.0 |
| Aloe leaf juice | 1.0 |
| Phase B | |
| Alcohol | 10 |
| Tetrahydro curcumin | 0.05 |
| Rosemary | 0.1 |
| PHMB | 0.12 |
| Cinnamon bark oil | 0.05 |
| Lemon grass oil | 0.01 |
| Clove bud oil | 0.03 |
| Peppermint | 0.05 |
| Hydrolite 5 | 0.5 |
| Solubilizer | 0.5 |
| Polysorbate 80 | 1.0 |

### 10.2 Testing Method

The antibacterial activities of oral rinses OCP-G1*, OCP-G2*, OCP-AIF* and commercially available GUM oral rinse (which contains 0.12 percent chlorhexidine) were measured after one minute contact with colonized bacteria on agar plates. For this test, 0.3 ml of microbial cultures of 10⁵ cfu/ml were seeded on Sabouraud Dextrose Broth Plate (SDB) and incubated for 4 hours at 37° C. in order for the pathogens to tightly adhere and colonize the agar surface. 0.3 ml of oral rinses or PBS (for the control sample) were then added and spread onto various plates. After 1 minute, 1 ml drug inactivating media was added to the plate and swirled to cover the whole plate. The plates were then incubated at 37° C. for 24 hours. The colony counts on the plate were enumerated.

### 10.3 Results

The antimicrobial activities of oral rinses OCP-G1*, OCP-G2*, OCP-AIF* and GUM against S. aureus and C. albicans, measured as set forth above, are presented in Table 6

TABLE 6
Log₁₀ reduction from control growth

| Organism | OCP-G1* | OCP-G2* | OCP-A1F* | GUM |
|---|---|---|---|---|
| S. aureus | 5.0 | 5.0 | 5.0 | 3.0 |
| C. albicans | 5.0 | 5.0 | 5.0 | 1.0 |

### 11. EXAMPLE 7

### 11.1 Formulation Tested

In a specific non-limiting embodiment, an anti-inflammatory, anti-irritant topical gel is provided comprising the following ingredients:

| Ingredients | % w/w |
|---|---|
| Water | 10.9 |
| Oat Powder | 1.5 |
| Tetrahydroeurcumanoid | 0.1 |
| PHMB | 0.1 |
| Lemon/citrus extract | 0.15 |
| Sym glucan (Beta glucan) | 0.75 |
| Dragocalm (Oat Kernel extract) | 0.5 |
| Chitidine 222 (Chitosan succinate) | 1.5 |
| portuluca extract | 0.5 |
| Propylene glycol | 10 |
| Octanediol | 0.8 |
| Hydroxypropyl methyl- | 0.1 |
| cellulose stearoxy ether (Sangelose) | |
| Aloe Gel | 40 |
| Natutal petrolatum | 30 |
| Pomegranate oil | 0.3 |
| Cinnamon oil | 0.1 |
| Lemon grass oil | 0.1 |
| Rosemary oil | 0.1 |
| Tocopherol | 0.5 |
| Natural emulsifier | 2.0 |
| (Glyceryl oleate citrate, Caprylic/ | |
| capric triglyceride) | |

### 11.2 Testing Method

The above gel was anecdotally tested by subjects for its effect on minor burn, mosquito bite, prickly heat, or dry skin; the results are subjective.

### 11.3 Results

Table 7A shows the subjective effects of the gel on blister formation and redness after minor burn in three subjects (denoted #1, #2 and #3).

TABLE 7A

| | Before | 10 min after | 30 min after |
|---|---|---|---|
| Volunteer # | application | application | application |
| 1 | +++ | + | − |
| 2 | +++ | + | − |
| 3 | +++ | − | − |

+++ Severe Redness
++ moderate redness
+ mild redness
− no redness

Table 7B shows the subjective effects of the gel on itch resulting from a mosquito bite in three subjects (denoted #4, #5, and #6).

TABLE 7B

| | Before | 5 minutes |
|---|---|---|
| Volunteer # | application | after application |
| 4 | +++ | − |
| 5 | +++ | − |
| 6 | +++ | − |

+++ Severe itching
++ moderate itching
+ mild itching
− no itching

Table 7C shows the subjective effects of the gel on itch resulting from prickly heat rash in three subjects (denoted #7, #8, and #9).

TABLE 7C

| | Before | 5 min after | 15 min after |
|---|---|---|---|
| Volunteer # | application | application | application |
| 7 | +++ | + | − |
| 8 | +++ | + | − |
| 9 | +++ | + | − |

+++ Severe itching
++ moderate itching
+ mild itching
− no itching

Table 7D shows the subjective effects of the gel on itch resulting from dry skin in three subjects (denoted #10, #11, and #12).

TABLE 7D

| | Before | 5 min after | 15 min after |
|---|---|---|---|
| Volunteer # | application | application | application |
| 10 | +++ | + | − |
| 11 | +++ | + | − |
| 12 | +++ | + | − |

+++ Severe itching
++ moderate itching
+ mild itching
− no itching

Table 7E shows the results of antimicrobial evaluation of a gel having a slightly different formula but having the same concentrations of tetrahydrocurcumanoid, PHMB, lemon/citrus extract, pomegranate oil, cinnamon oil, lemongrass oil, and rosemary oil (the formulation tested for Table 7E had witch hazel extract rather than portulaca extract), and compares that gel to a commercially available 2% miconazole cream.

TABLE 7E
Zone of Inhibition (mm)

| | P. aeuroginosa | S. aureus | C. albicans |
|---|---|---|---|
| Test Gel | 8.0 | 12.5 | 18 |
| 2% miconazole | 8.0 | 13 | 15 |
| cream | | | |

### 12. EXAMPLE 8

### 12.1 Formulations Tested

A hand sanitizer foam was tested comprising the following ingredients (“FOAM BSAM-4-1”):

| Ingredients | % w/w |
|---|---|
| Water | 88.865 |
| Hydroxypropyl methyl- | 0.025 |
| cellulose stearoxy ether (Sangelose) | |
| Solubulizer (PEG-40 hydrogented | 2.0 |
| Castor oil, Trideceth 9, water) | |
| Plantasol | 2.0 |
| Lemon extract | 0.5 |
| Octanediol | 1.0 |
| Phenyl ethanol | 0.3 |
| Lernongrass oil | 0.1 |
| Aloe leaf juice | 1.0 |
| BAC | 0.13 |
| PHMB | 0.06 |
| Thymol | 0.07 |
| Cinnamon oil | 0.1 |
| Dipropylene glycol | 1.0 |
| Cetyl pyridium chloride | 0.25 |
| Citrus extract | 0.3 |
| Bisabolol | 0.05 |
| Ethoxy diglycol (Edicol) | 1.0 |
| Dow coming cosmetic wax 2501 | 0.75 |
| lsododecane and isononanoate | 0.50 |
| (Clerocast 550) | |

A hand sanitizer lotion was tested having the following ingredients (“LOTION BASM-4-1”):

| Ingredients | % w/w |
|---|---|
| Water | 83.81 |
| Hydroxypropyl methyl-cellulose | 0.2 |
| stearoxy ether | |
| (Sangelose) | |
| Cetyl pyridinium chloride | 0.25 |
| Propylene glycol | 1.0 |
| Glycerin | 3.0 |
| Incroquat TMS | 1.5 |
| Polowax NF | 0.5 |
| Stearyl alcohol | 0.5 |
| Zinc oxide | 0.3 |
| Silicone fluid (Dimethicone 200/350) | 2.0 |
| Aloe juice | 1.0 |
| Bisabolol | 0.05 |
| Grape fruit seed extract | 0.25 |
| Lemon extract | 0.25 |
| Phenyl Ethanol | 0.3 |
| Thymol | 0.05 |
| BAC | 0.13 |
| PHMB | 0.06 |
| Lemongrass oil | 0.05 |
| Pomegranate oil | 0.4 |
| Almond oil | 1.0 |
| 2-methyl-1,3-propanediol | 1.0 |
| (Solidol) | |
| Lactic acid | 0.1 |
| Octanediol | 1.0 |
| Cinnamon leaf oil | 0.05 |
| Dow corning 2501 | 0.75 |
| Clerocast 550 | 0.50 |
| Adjust pH to 4.0-4.5 | |

### 12.2 Testing Method

A rapid tube dilution (15 second time kill) method was used. 0.9 ml of the test sample was inoculated with bacterial culture 10⁷ CFU/ml). After 15 seconds drug neutralizing fluid (DNF) was added to stop antimicrobial action. Serial dilutions were made and subcultured on Trypticase Soy Agar plates, incubated for 24-48 hours. Phosphate buffer saline was used as control.

### 12.3 Results

The results of antimicrobial testing are presented in Table 8.

TABLE 8
Log reduction from control growth*

| | Soap: 0.3% | Soap: 0.3% | FOAM | LOTION |
|---|---|---|---|---|
| Organism | Triclosan | PCMX | BSAM-4-1 | BSAM-4-I |
| S. aureus | 3.2 | 2.7 | 3.0 | 3.5 |
| P. aeruginosa | 2.0 | 2.8 | 6.1 | 6.1 |
| E. coli | 2.5 | 2.8 | ×6.2 | 5.2 |
| MRSA | 2.6 | 2.6 | 3.3 | 5.0 |

*Control growth ranged from 5 × 10⁵ to 1 × 10⁶

### 13. EXAMPLE 9

### 13.1 Formulation Tested

A “SynBotanica First Aid Cream-1” was tested having the following ingredients:

| Ingredients | % w/w |
|---|---|
| PHMB | 0.06 |
| Lemon extract | 0.15 |
| Cinnamon bark oil | 0.1 |
| Pomegranite seed oil | 0.25 |
| Portulaca extract | 0.5 |
| Aloe gel | 40 |
| Natural petrolactum | 20 |
| 1,3 propanediol | 7 |
| Oat flour | 2 |
| Water | 29.64 |
| Zinc oxide | 0.3 |
| Germal plus | 0.3 |

### 13.2 Testing Methods

Trypticase soy agar plates were seeded with 0.1 ml of 10⁸ CFU bacteria and 10⁷ C. albicans. Four wells of 0.7 cm were made on the plate using a cork borer. Each well was filled with 0.1 g of the cream, and the plates were incubated for 24 hrs at 37° C. The zones of inhibition were measured.

### 13.3 Results

The results of antimicrobial testing are shown in Table 9.

TABLE 9

| Organisms | Zone of inhibition (min) |
|---|---|
| S. aureus | 14 |
| P. aeruginosa | 12 |
| C. albicans | 19 |

### 14. EXAMPLE 10

This example demonstrates that botanical extracts such as vanilla extract and lemon extract exhibit synergistic antibacterial activity with PHMB. However, chlorhexidine, which is another biguanide, did not show such synergism.

14.1 Testing Method

Synergistic effect of botanical extracts and biguanides (Rapid kill method (108 S. aureus)): The antimicrobial effect of an oral rinse base, containing the ingredients described in table 10 below, were tested against a high concentration of bacteria. In a sterile culture tube, 0.1 ml of S. aureus (108 cfu/ml) was treated with 0.9 ml of test mouthrinse for 15 seconds. The reaction was stopped with Drug neutralizing Fluid (DNF) and then further diluted with the same media. 0.5 ml of the dilution was then plated on Tripticase Soy Agar (TSA) and incubated at 37° C. overnight. The colony counts were determined and the log 10 reduction from the control (phosphate-buffered saline (PBS) inoculated with the same culture) was calculated. This test shows the efficacy of the following ingredients in oral rinses in inactivating oral pathogens after a 15 second contact time.

### 14.1.1 Results

TABLE 10
Synergistic effect of botanical extracts and biguanides
(Rapid kill method (10⁸ cfu/ml S. aureus)).

| Name | Log₁₀ | |
|---|---|---|
| Reduction | Growth | Log₁₀ |
| Control | 7.29 | 0.0 |
| CHX | 5.60 | 1.69 |
| (0.06%) | | |
| PHMB | 4.58 | 2.71 |
| (0.06%) | | |
| Citrus extract | 7.01 | 0.28 |
| (0.6%) | | |
| Vanilla extract | 7.12 | 0.17 |
| Citrus extract | 6.81 | 0.48 |
| (0.3%) + Vanilla extract (0.3%) | | |
| Vanilla extract | 1.98 | 5.31 |
| (0.3%) + PHMB (0.06%) | | |
| Citrus extract | 2.1 | 5.19 |
| (0.3%) + PHMB (0.06%) | | |
| Citrus extract | 5.56 | 1.73 |
| (0.3%) + CHX (0.06%) | | |
| Vanilla extract | 5.68 | 1.61 |
| (0.3%) + CHX (0.06%) | | |
| Citrus extract | 5.43 | 1.86 |
| (0.3%) + Vanilla extract (0.3%) + CHX (0.06%) | | |
| Citrus extract | 1.99 | 5.30 |
| (0.3%) + Vanilla extract (0.3%) + PHMB (0.06%) | | |

### 14.1.2 Conclusion

Botanical extracts exhibit synergism specifically with PHMB.

14.2 Additional Examples of Oral Compositions are as Follows:

### General Formulation

| General Main ingredients | % w/w Range |
|---|---|
| 1. Botanical Extracts | 0.1-0.6 |

Vanilla extract, Lemon extract, Grape fruit seed extract.

| | |
|---|---|
| 2. Synthetic antimicrobials | |
| PHMB | 0.01-0.3 |

With or without one of the following quaternary ammonium compounds

| | |
|---|---|
| Benzalkonium chloride (BAC) | 0.08-0.13 |
| Benzethonium chloride (BZT) | 0.08-0.2 |
| Cetyl pyridinium chloride | 0.02-0.5 |
| 3. Essential oils/Ingredients | 0.01-0.5 |

Lemon grass oil, Lemon oil, Fennel seed oil, Orange oil, Basil oil,
Cinnamon oil, Thyme oil Clove oil, Pine oil, Cedar wood oil. Thymol,
Farnesol, Bisabolol

| | |
|---|---|
| 4. Alkanediols | 0.0-5.0 |

Propanediol, pentanediol, hexanediol, heptanediol, octanediol, decanediol
or nonanediol.

| | |
|---|---|
| 5. Organic acids | (0.05-0.5) |
| Benzoic acid, Lactic acid, Citric acid. | |
| 6. Solvents/Solubilizer | |
| Alcohol | 0-80 |
| Phenyl ethanol | 0-1.0 |
| PEG-40 Hydrogenated Castor oil, | 0-2.0 |
| Trideceth-9, Propylene glycol, Water (Aqua) | |
| PEG-40 Hydrogenated Castor oil, | 0.-2.0 |
| Trideceth-9, Water (Aqua) | |
| Caprylyl capryl Glucoside (Plantasol) | 0-2.0 |

Optionally, anti-irritants selected from the group Brahmi extracts, Red sandal wood extracts, Curcumin compounds, Aloe products, Bisabolol, Rosemary oil, Chalmogra oil, oat powder/extract, Beta glucan, Chitosan. Calendula oil, Portulaca extract, Witch hazel extract, Pomegranate oil (0.1-2.0).

### Specific Formulations

### Oral Rinse (6B)

| Main ingredients | % w/w |
|---|---|
| Vanilla extract | 0.6 |
| Thymol | 0.05 |
| Cinnamon bark oil | 0.05 |
| PHMB | 0.1 |
| Benzoic acid | 0.1 |
| Other ingredients | |
| Water | 76.45 |
| Pluronic F-127 | 1.0 |
| Sorbitol | 5.0 |
| Xylitol | 0.5 |
| Zinc salicylate | 0.1 |
| Glycerin | 12.0 |
| Zemea (1,2-propanediol) | 4.0 |
| Peppermint oil | 0.05 |

### Oral Rinse (7B)

| Main ingredients | % w/w |
|---|---|
| Vanilla extract | 0.6 |
| Thymol | 0.05 |
| Cinnamon bark oil | 0.05 |
| PHMB | 0.06 |
| Benzoic acid | 0.1 |
| Other ingredients | |
| Water | 76.45 |
| Pluronic F-127 | 1.0 |
| Sorbitol | 5.0 |
| Xylitol | 0.5 |
| Zinc salicylate | 0.1 |
| Glycerin | 12.0 |
| Zemea (1,2-propanediol) | 4.0 |
| Peppermint oil | 0.05 |

### Oral Rinse (8B)

| Main ingredients | % w/w |
|---|---|
| Vanilla extract | 0.6 |
| Thymol | 0.05 |
| Cinnamon bark oil | 0.05 |
| PHMB | 0.03 |
| Benzoic acid | 0.1 |
| Other ingredients | |
| Water | 76.45 |
| Pluronic F-127 | 1.0 |
| Sorbitol | 5.0 |
| Xylitol | 0.5 |
| Zinc salicylate | 0.1 |
| Glycerin | 12.0 |
| Zemea (1,2-propanediol) | 4.0 |
| Peppermint oil | 0.05 |

Oral rinse 15BCF

| Main ingredients | % w/w |
|---|---|
| Vanilla extract | 0.3 |
| Thymol | 0.05 |
| Cinnamon bark oil | 0.01 |
| PHMB | 0.1 |
| Benzoic acid | 0.1 |
| Basil oil | 0.01 |
| Fennel seed oil | 0.05 |
| Other ingredients | |
| Water | 74.73 |
| Pluronic F-127 | 1.0 |
| Sorbitol | 5.0 |
| Xylitol | 0.5 |
| Zinc salicylate | 0.1 |
| Glycerin | 12.0 |
| Zemea (1,2-propanediol) | 4.0 |
| Propylene glycol | 2.0 |
| Peppermint oil | 0.05 |

Oral rinse A2

| Main ingredients | % w/w |
|---|---|
| Vanilla extract | 0.3 |
| Thymol | 0.05 |
| Cinnamon bark oil | 0.01 |
| PHMB | 0.02 |
| Benzoic acid | 0.1 |
| Basil oil | 0.01 |
| Fennel seed oil | 0.05 |
| Tetrahydrocurcumin | 0.05 |
| Other ingredients | |
| Water | 64.76 |
| Pluronic F-127 | 1.0 |
| Sorbitol | 5.0 |
| Xylitol | 0.5 |
| Zinc salicylate | 0.1 |
| Glycerin | 12.0 |
| Alcohol | 10.0 |
| Zemea (1,2-propanediol) | 4.0 |
| Propylene glycol | 2.0 |
| Peppermint oil | 0.05 |

### Oral Rinse 16BCF

| Main ingredients | % w/w |
|---|---|
| Tetrahydrocurcumin | 0.05 |
| Cinnamon bark oil | 0.01 |
| Basil oil | 0.01 |
| Fennel seed oil | 0.05 |
| Thymol | 0.05 |
| Vanilla extract | 0.3 |
| PHMB | 0.1 |
| Other ingredients | |
| Water | 64.28 |
| Zemea | 4.0 |
| Alcohol | 10.0 |
| Pluronic F 127 | 1.0 |
| Benzoic acid | 0.1 |
| Glycerin | 12.0 |
| Zinc salicylate | 0.1 |
| Sorbitol | 5.0 |
| Xylitol | 0.5 |
| Propylene glycol | 2.0 |
| Peppermint oil | 0.05 |

### Oral Rinse 17BCF.1

| Main ingredients | % w/w |
|---|---|
| Cinnamon bark oil | 0.01 |
| Basil oil | 0.01 |
| Fennel seed oil | 0.1 |
| Thymol | 0.05 |
| Vanilla extract | 0.3 |
| PHMB | 0.1 |
| Other ingredients | |
| Water | 74.18 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 1.0 |
| Peppermint oil | 0.05 |
| Caprylyl capryl Glucoside (Plantasol) | 0.5 |

### Oral Rinse 17BCF.2

| Main ingredients | % w/w |
|---|---|
| Cinnamon bark oil | 0.01 |
| Basil oil | 0.01 |
| Fennel seed oil | 0.1 |
| Thymol | 0.1 |
| Vanilla extract | 0.3 |
| PAPB | 0.1 |
| Other ingredients | |
| Water | 74.18 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 4.0 |
| Xylitol | 2.0 |
| Peppermint oil | 0.05 |
| Caprylyl capryl Glucoside (Plantasol) | 0.5 |
| Solubilizer (611674) | 0.25 |

### Oral Rinse 17BCF-1

| Main ingredients | % w/w |
|---|---|
| Cinnamon bark oil | 0.01 |
| Basil oil | 0.01 |
| Fennel seed oil | 0.1 |
| Thymol | 0.075 |
| Vanilla extract | 0.3 |
| PHMB | 0.1 |
| Other ingredients | |
| Water | 74.18 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 1.0 |
| Peppermint oil | 0.05 |
| Caprylyl capryl Glucoside (Plantasol) | 0.5 |

### Oral Rinse 17BCF-2

| Main ingredients | % w/w |
|---|---|
| Cinnamon bark oil | 0.01 |
| Basil oil | 0.01 |
| Fennel seed oil | 0.1 |
| Thymol | 0.05 |
| Vanilla extract | 0.3 |
| PAPB | 0.10 |
| Other ingredients | |
| Water | 73.68 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 3.0 |
| Xylitol | 2.0 |
| Peppermint oil | 0.05 |
| Caprylyl capryl Glucoside (Plantasol) | 0.5 |

### Oral Rinse 17BCF-2.2

| Main ingredients | % w/w |
|---|---|
| Cinnamon bark oil | 0.01 |
| Basil oil | 0.01 |
| Fennel seed oil | 0.1 |
| Thymol | 0.1 |
| Vanilla extract | 0.3 |
| PHMB | 0.1 |
| Other ingredients | |
| Water | 74.18 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 4.0 |
| Xylitol | 2.0 |
| Peppermint oil | 0.05 |
| Caprylyl capryl Glucoside (Plantasol) | 0.5 |

### Oral Rinse 17BCF-2A

| Main ingredients | % w/w |
|---|---|
| Cinnamon bark oil | 0.01 |
| Basil oil | 0.01 |
| Fennel seed oil | 0.1 |
| Thymol | 0.1 |
| Vanilla extract | 0.3 |
| PAPB | 0.10 |
| Other ingredients | |
| Water | 73.68 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 4.0 |
| Xylitol | 2.0 |
| Peppermint oil | 0.05 |
| Caprylyl capryl Glucoside (Plantasol) | 0.5 |

### Oral Rinse 17BCF-1A

| Main ingredients | % w/w |
|---|---|
| Cinnamon bark oil | 0.01 |
| Basil oil | 0.01 |
| Fennel seed oil | 0.1 |
| Thymol | 0.075 |
| Vanilla extract | 0.3 |
| PAPB | 0.1 |
| Other ingredients | |
| Water | 74.18 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 1.0 |
| Peppermint oil | 0.05 |
| Caprylyl capryl Glucoside (Plantasol) | 0.5 |

### Oral Rinse 18BCF.1

| | % w/w |
|---|---|
| Main ingredients | |
| Cinnamon bark oil | 0.01 |
| Basil oil | 0.01 |
| Fennel seed oil | 0.1 |
| Thymol | 0.05 |
| Vanilla extract | 0.3 |
| PHMB | 0.12 |
| Other ingredients | |
| Water | 74.16 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |
| Caprylyl capryl Glucoside (Plantasol) | 0.5 |

### Oral Rinse 18BCF.2

| | % w/w |
|---|---|
| Main ingredients | |
| Cinnamon bark oil | 0.01 |
| Basil oil | 0.01 |
| Fennel seed oil | 0.1 |
| Thymol | 0.05 |
| Vanilla extract | 0.3 |
| PAPB | 0.12 |
| Other ingredients | |
| Water | 74.16 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |
| Caprylyl capryl Glucoside (Plantasol) | 0.5 |

### Oral Rinse 19BCF-1

| | % w/w |
|---|---|
| Main ingredients | |
| Cinnamon bark oil | 0.01 |
| Basil oil | 0.01 |
| Fennel seed oil | 0.1 |
| Thymol | 0.075 |
| Vanilla extract | 0.3 |
| PHMB | 0.08 |
| Other ingredients | |
| Water | 74.2 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 1.0 |
| Peppermint oil | 0.05 |
| Caprylyl capryl Glucoside (Plantasol) | 0.5 |

### Oral Rinse 19BCF-1A:

| | % w/w |
|---|---|
| Main ingredients | |
| Cinnamon bark oil | 0.01 |
| Basil oil | 0.01 |
| Fennel seed oil | 0.1 |
| Thymol | 0.05 |
| Vanilla extract | 0.3 |
| PAPB | 0.08 |
| Other ingredients | |
| Water | 73.68 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 1.0 |
| Peppermint oil | 0.05 |
| Caprylyl capryl Glucoside (Plantasol) | 0.5 |

### Oral Rinse 19BCF-2A

| | % w/w |
|---|---|
| Main ingredients | |
| Cinnamon bark oil | 0.01 |
| Basil oil | 0.01 |
| Fennel seed oil | 0.1 |
| Thymol | 0.05 |
| Vanilla extract | 0.3 |
| PAPB | 0.08 |
| Other ingredients | |
| Water | 73.7 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 4.0 |
| Xylitol | 2.0 |
| Peppermint oil | 0.05 |
| Caprylyl capryl Glucoside (Plantasol) | 0.5 |

### Oral Rinse 19BCF.2

| | % w/w |
|---|---|
| Main ingredients | |
| Cinnamon bark oil | 0.01 |
| Basil oil | 0.01 |
| Fennel seed oil | 0.1 |
| Thymol | 0.1 |
| Vanilla extract | 0.3 |
| PAPB | 0.08 |
| Other ingredients | |
| Water | 74.2 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 4.0 |
| Xylitol | 2.0 |
| Peppermint oil | 0.05 |
| Caprylyl capryl Glucoside (Plantasol) | 0.5 |

### Oral Rinse 19BCF-2

| | % w/w |
|---|---|
| Main ingredients | |
| Cinnamon bark oil | 0.01 |
| Basil oil | 0.01 |
| Fennel seed oil | 0.1 |
| Thymol | 0.05 |
| Vanilla extract | 0.3 |
| PAPS | 0.08 |
| Other ingredients | |
| Water | 73.7 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 3.0 |
| Xylitol | 2.0 |
| Peppermint oil | 0.05 |
| Caprylyl capryl Glucoside (Plantasol) | 0.5 |

### Oral Rinse A3.1

| | % w/w |
|---|---|
| Main ingredients | |
| Tetrahydrocurcumin | 0.05 |
| Cinnamon bark oil | 0.01 |
| Basil oil | 0.01 |
| Fennel seed oil | 0.1 |
| Thymol | 0.05 |
| Vanilla extract | 0.3 |
| PHMB | 0.02 |
| Other ingredients | |
| Water | 66.71 |
| Zemea | 4.0 |
| Alcohol | 10.0 |
| Pluronic F 127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |

### Oral Rinse A3.2

| | % w/w |
|---|---|
| Main ingredients | |
| Cinnamon bark oil | 0.01 |
| Basil oil | 0.01 |
| Fennel seed oil | 0.1 |
| Thymol | 0.05 |
| Vanilla extract | 0.3 |
| PAPB | 0.02 |
| Other ingredients | |
| Water | 66.71 |
| Zemea | 4.0 |
| Alcohol | 10.0 |
| Pluronic F 127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |
| Tetrahydrocurcumin | 0.05 |

### Oral Rinse 17A.1

| | % w/w |
|---|---|
| Main ingredients | |
| Tetrahydrocurcumin | 0.05 |
| Cinnamon bark oil | 0.01 |
| Basil oil | 0.01 |
| Fennel seed oil | 0.1 |
| Thymol | 0.05 |
| Vanilla extract | 0.3 |
| PHMB | 0.1 |
| Other ingredients | |
| Water | 76.73 |
| Zemea | 4.0 |
| Propylene glycol | 2.0 |
| Alcohol | 10.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |

### Oral Rinse 17A.2

| | % w/w |
|---|---|
| Main ingredients | |
| Cinnamon bark oil | 0.01 |
| Basil oil | 0.01 |
| Fennel seed oil | 0.1 |
| Thymol | 0.05 |
| Vanilla extract | 0.3 |
| PAPB | 0.1 |
| Other ingredients | |
| Water | 64.63 |
| Zemea | 4.0 |
| Propylene glycol | 2.0 |
| Alcohol | 10.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |
| Tetrahydrocurcumin | 0.05 |

### Oral Rinse 17BCF-C.1

| | % w/w |
|---|---|
| Main ingredients | |
| Cinnamon bark oil | 0.01 |
| Clove bud oil | 0.01 |
| Basil oil | 0.01 |
| Fennel seed oil | 0.1 |
| Thymol | 0.05 |
| Vanilla extract | 0.3 |
| PHMB | 0.1 |
| Other ingredients | |
| Water | 74.67 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |

### Oral Rinse 17BCF-C.2

| | % w/w |
|---|---|
| Main ingredients | |
| Cinnamon bark oil | 0.01 |
| Clove bud oil | 0.01 |
| Basil oil | 0.01 |
| Fennel seed oil | 0.1 |
| Thymol | 0.05 |
| Vanilla extract | 0.3 |
| PAPB | 0.1 |
| Other ingredients | |
| Water | 74.67 |
| Zemea | 4.0 |
| Propylene glycol | 2.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |

### Oral Rinse 17A-C.1

| | % w/w |
|---|---|
| Main ingredients | |
| Tetrahydrocurcumin | 0.05 |
| Cinnamon bark oil | 0.01 |
| Clove bud oil | 0.01 |
| Basil oil | 0.01 |
| Fennel seed oil | 0.1 |
| Thymol | 0.05 |
| Vanilla extract | 0.3 |
| PHMB | 0.1 |
| Other ingredients | |
| Water | 64.62 |
| Zemea | 4.0 |
| Propylene glycol | 2.0 |
| Alcohol | 10.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |

### Oral Rinse 17A-C.2

| | % w/w |
|---|---|
| Main ingredients | |
| Cinnamon bark oil | 0.01 |
| Clove bud oil | 0.01 |
| Basil oil | 0.01 |
| Fennel seed oil | 0.1 |
| Thymol | 0.05 |
| Vanilla extract | 0.3 |
| PAPS | 0.1 |
| Other ingredients | |
| Water | 64.67 |
| Zemea | 4.0 |
| Propylene glycol | 2.0 |
| Alcohol | 10.0 |
| Pluronic F-127 | 1.0 |
| Benzoic acid | 0.1 |
| Glycerin | 12.0 |
| Zinc salicylate | 0.1 |
| Sorbitol | 5.0 |
| Xylitol | 0.5 |
| Peppermint oil | 0.05 |
| Tetrahydrocurcumin | 0.05 |

### 14.2.1 Testing Method

Method 2: Antibacterial Activity of Oral Rinses after One Minute Contact with 6 h Colonized Bacteria on the Agar Plates.

For this test, 0.3 ml of microbial cultures of 10⁶ cfu/ml were seeded on Trypticase Soy agar Plate (TSA) and incubated for 6 hours at 37° C. in order for the pathogens to tightly adhere and colonize the agar surface. 0.5 ml of oral rinses and PBS (for the control sample) was then added and spread on the surface of the plates. After 1 minute, 1 ml drug inactivating media was added to the plate and swirled to cover the whole plate and plates were then incubated at 37° C. for 24 hours. The colony counts on the plate were enumerated.

### 14.2.2 Results

Candida albicans (0.3 ml of 10⁶ cfu/ml) incubated for 6 h and treated with 0.5 ml of following oral rinse.

Group: C. albicans

| | Logl0 growth | Log10 Reduction |
|---|---|---|
| Control | 6.12 | 0.0 |
| 6B | 0.2 | 5.92 |
| 15BCF | 0.2 | 5.92 |
| A2 | 0.2 | 5.92 |
| 16BCF | 0.12 | 6.00 |
| Gum* | 3.12 | 3.0 |

*“GUM” Formulation”
Chlorhexidine Gluconate (0.12% w/w)
Water (Deionized)
Propylene glycol
Glycerin
Polyoxyl 40 hydrogenated castor oil
Potassium acesulfame
Mint flavor
FD & C Red #40
D & C Red # 33

### 14.2.3 Conclusion

Botanical extracts combined with PHMB exhibit antimicrobial effects against C. albicans.

### 15. EXAMPLE 11

15.1 Examples of Topical Antimicrobial Compositions Comprising Botanical Extracts, PHMB and Essential Oils (or Active Constituents Thereof) are Described Below.

### Hand Sanitizer Aqueous Foam

| Main ingredients | % w/w |
|---|---|
| Octanediol | 0.8 |
| Lactic acid | 0.2 |
| Alcohol | 10.0 |
| Lemon oil | 0.10 |
| Lemon grass oil | 0.1 |
| Orange oil | 0.1 |
| Thymol | 0.05 |
| Vanilla extract | 0.3 |
| Cosmocil (PHMB) | 1.0 (0.2) |
| Benzalkonium chloride | 0.13 |
| Benzoic acid | 0.2 |
| Phenyl ethanol | 0.3 |
| Other ingredients | % w/w |
| Water | 81.69 |
| Glycerin | 1.0 |
| Hydroxypropyl methyl | 0.03 |
| cellulose stearoxy ether | |
| (Sangelose) 90 L | |
| Solubilizer (PEG-40 hydrogenated | 1.0 |
| Castor oil, Trideceth 9, Water) | |
| Plantasol | 1.0 |
| 2-Methyl-1,3-propanediol | 0.75 |
| (Solidol MP-D) | |
| Ethoxy diglycol | 0.5 |
| Isododocane and isononanoate | 0.5 |
| Silicone KF-6043 | 0.25 |

### Aqueous Hand Sanitizer Lotion-1 (Syn-Botanica Lotion-1)

| Main ingredients | % w/w |
|---|---|
| Octanediol | 0.8 |
| Lactic acid | 0.2 |
| Alcohol | 10.0 |
| Lemon oil | 0.10 |
| Lemon grass oil | 0.1 |
| Orange oil | 0.1 |
| Thymol | 0.05 |
| Vanilla extract | 0.3 |
| Cosmocil (PHMB) | 1.0 (0.2) |
| Benzalkonium chloride | 0.13 |
| Benzoic acid | 0.2 |
| Phenyl ethanol | 0.3 |
| Other ingredients | |
| Water | 74.17 |
| Hydroxypropyl methyl-cellulose | 0.2 |
| stearoxy ether (Sangelose 90 L) | |
| Glycerin | 1.0 |
| Incroquat TMS | 1.5 |
| Polowax NF | 1.0 |
| Stearyl alcohol | 0.8 |
| Zinc oxide | 0.4 |
| Silicone fluid (Dimethicone200/350) | 2.0 |
| Almond oil | 1.5 |
| Lactic acid | 0.1 |
| Aloe juice | 2.0 |
| Bisabolol | 0.05 |
| Zemea | 1.0 |
| Silicone KF 6043 | 1.0 |
| Adjust pH to 4.0-4.5 | |

### Aqueous Hand Sanitizer Lotion-2

| Main ingredients | % w/w |
|---|---|
| Octanediol | 0.8 |
| Lactic acid | 0.2 |
| Alcohol | 10.0 |
| Lemon oil | 0.10 |
| Lemon grass oil | 0.1 |
| Orange oil | 0.1 |
| Thymol | 0.05 |
| Vanilla extract | 0.3 |
| Cosmocil (PHMB) | 1.0 (0.2) |
| Benzalkonium chloride | 0.13 |
| Benzoic acid | 0.2 |
| Phenyl ethanol | 0.3 |
| Other ingredients | |
| Water | 69.77 |
| Oat powder | 1.0 |
| Hydroxypropyl methyl-cellulose | 0.2 |
| stearoxy ether (Sangelose) | |
| Glycerin | 4.0 |
| Incroquat Behenyl TMS | 2.0 |
| Polowax NF | 1.0 |
| Stearyl alcohol | 0.8 |
| Zinc oxide | 0.6 |
| Silicone fluid (Dimethicone200/350) | 2.0 |
| Almond oil | 2.0 |
| Lactic acid | 0.3 |
| Aloe juice | 2.0 |
| Bisabolol | 0.05 |
| Zemea | 1.0 |
| Adjust pH to 4.0-4.5 | |

### Hand Disinfectant Soap-1 (Syn-Botanica Soap-1)

| Main ingredients | % w/w |
|---|---|
| Octanediol | 0.5 |
| Citric acid | 0.5 |
| Alcohol | 10.0 |
| Lemon oil | 0.1 |
| Lemon grass oil | 0.1 |
| Orange oil | 0.1 |
| Thymol | 0.05 |
| Vanilla extract | 0.3 |
| Cosmocil (PHMB) | 1.0 (0.2) |
| Benzalkonium chloride | 0.13 |
| Benzoic acid | 0.2 |
| Other ingredients | |
| Water | 71.97 |
| Polyox N60 K | 0.15 |
| Polyquaternium 10 ( ucare JR 30) | 0.2 |
| Pluronic surfactant F 87 | 2.0 |
| Cocoamido propyl betaine | 6.0 |
| Purasil Mild | 1.5 |
| Phenoxy ethanol | 0.7 |
| Aloe Juice | 1.0 |
| Glycerin | 1.0 |
| Solubulizer (611624) | 0.5 |
| Plantasol | 1.0 |
| Silicone fluid (Dimethicone200/350) | 1.0 |
| pH 4.0 | |

### Hand Disinfectant Soap-2

| Main ingredients | % w/w |
|---|---|
| Octanediol | 0.5 |
| Citric acid | 0.5 |
| Alcohol | 10.0 |
| Lemon oil | 0.1 |
| Lemon grass oil | 0.1 |
| Orange oil | 0.1 |
| Thymol | 0.05 |
| Vanilla extract | 0.3 |
| Cosmocil (PHMB) | 1.0 (0.2) |
| Benzalkonium chloride | 0.13 |
| Benzoic acid | 0.2 |
| Other ingredients | |
| Water | 69.32 |
| Polyox N60 K | 0.2 |
| Polyquaternium 10 ( ucare JR 30) | 0.3 |
| Pluronic surfactant F 87 | 1.0 |
| Cocoamido propyl betaine | 6.0 |
| Mackam BC 39 | 3.0 |
| Purasil Mild | 2.0 |
| Phenoxy ethanol | 0.7 |
| Aloe Juice | 1.0 |
| Glycerin | 2.0 |
| Solubulizer (611624) | 0.5 |
| Plantasol | 1.0 |
| pH 4.0 | |

### 15.1.1 Testing Method

Rapid (15 second) and broad spectrum antibacterial efficacy of Synbotanica soap and lotion were tested against various microorganisms. The ASTM E 2783-11 method for testing the antimicrobial efficacy using a time kill procedure was used. The rapid antibacterial activity (15 seconds exposure) of the SynBotanica soap and lotion was tested against various organisms listed in below tables and given with results.

Method 3: In a sterile culture tube, 0.1 ml of bacterial culture (106 cfu/ml) was treated with 0.9 ml of test sample for 15 seconds. The reaction was stopped with Drug neutralizing Fluid (DNF) and then further diluted with the same media. 0.5 ml of the dilution was then plated on Tripticase Soy Agar (TSA) and incubated at 37° C. overnight. The colony counts were determined and the log 10 reduction from the control (phosphate-buffered saline (PBS) inoculated with the same culture) was calculated.

### 15.1.2 Results

Rapid and Broad Spectrum Antibacterial Efficacy of Triclosan free Syn-Botanica Soap-1 (also herein referred to as Hand disinfectant Soap-1) and Syn-Botanica lotion-1 (also herein referred to as Aqueous Hand Sanitizer Lotion-1), are shown in the table below:

### Organism Log 10 Reduction in 15 Seconds)

| | | Syn-Botanica | Syn-Botanica |
|---|---|---|---|
| | Soap: 0.3%Triclosan | Soap-1 | lotion-1 |
| S. aureus | 3.2 | 4.0 | 3.95 |
| P. aeruginosa | 2.0 | 5.1 | 4.68 |
| E. coli | 2.5 | 5.2 | 4.89 |
| MRSA | 2.6 | 4.8 | 4.53 |
| E.aerogenes | 3.5 | 4.5 | 4.21 |

MRSA = Methicillin-resistant S. aureus; control growth ranged from 5 × 10⁵ to 1 × 10⁶ cfu/ml.

15.2 Additional Examples of Topical Compositions are as Follows:

Cow teat disinfectant solution

| Main ingredients | % w/w |
|---|---|
| PHMB | 0.3 |
| Vanilla extract | 0.3 |
| Octanediol | 0.7 |
| Rosemary oil | 0.1 |
| Other ingredients | |
| Aloe juice | 2.0 |
| Propylene glycol | 5.0 |
| Glycerine | 5.0 |
| Water | 86.6 |

### 16. EXAMPLE 12

Antibacterial activity against various bacteria after 1 minute exposure to oral rinse (OCP-1) containing PHMB and other oral rinses (Bacteria tested 10⁵ cfu/ml) was tested. Bacteria were grown on agar plates for 4 hours with the test composition. Results are shown in Table 11 below:

### Oral Rinse OCP-1:

| Ingredients | % w/w |
|---|---|
| Phase A (Other ingredients) | |
| Water | 83.09 |
| Xylitol | 0.25 |
| Sorbitol | 0.5 |
| Pluronic F127 | 0.5 |
| Lactic acid | 0.1 |
| Zinc salicylate | 0.1 |
| Glycerin | 7.0 |
| Propylene glycol | 1.0 |
| Zemea | 5.0 |
| Aloe leaf juice | 1.0 |
| Phase B (Main ingredients) | |
| Vanilla extract | 0.6 |
| PHMB | 0.12 |
| Cinnamon bark oil | 0.05 |
| Lemon grass oil | 0.01 |
| Clove bud oil | 0.03 |
| Peppermint | 0.05 |
| Hydrolite 5 | 0.5 |
| Polysorbate 80 | 0.1 |

TABLE 11
Log10 growth of organisms after exposure to antimicrobial compositions

| Microorganisms | Control | Listerine | PerioRx | OCP-1 |
|---|---|---|---|---|
| MRSA | 5 | 2.44 | 1.86 | 0 |
| S. aureus | 5 | 2.99 | 2.11 | 0 |
| C. albicans | 5 | 3.45 | 0.2 | 0 |
| P. aeruginosa | 5 | 2.76 | 4 | 0 |

### 17. EXAMPLE 13

Antibacterial activity against various bacteria after 1 minute exposure to oral rinses containing PHMB (0.1%, OCP-M) and GUM (10⁶ cfu/ml) was tested. Bacteria were grown on agar plates for 6 hours with the test composition. Results are shown in Table 12 below:

### Oral Rinse OCP-M

| Ingredients | % w/w |
|---|---|
| Lemon extract | 0.6 |
| Cinnamon bark oil | 0.05 |
| Thymol | 0.05 |
| Clove bud oil | 0.03 |
| Basil | 0.05 |
| Menthol | 0.05 |
| Spearmint oil | 0.04 |
| Eucalyptus | 0.03 |
| Lemon grass oil | 0.05 |
| Rosemary oil | 0.05 |
| PHMB | 0.06 |
| Peppermint oil | 0.03 |
| Water | 73.29 |
| Xylitol | 0.25 |
| Sorbitol | 0.75 |
| Pluronic F 087 | 0.1 |
| Zinc salicylate | 0.1 |
| Lactic acid | 0.1 |
| Glycerin | 7.0 |
| Zemea | 2.0 |
| Aloe leaf juice | 1.0 |
| Propylene glycol | 6.0 |
| Plantasol (Caprylyl capryl Glucoside) | 1.0 |

“GUM” Formulation
Chlorhexidine Gluconate (0.12% w/w)
Water (Deionized)
Propylene glycol
Glycerin
Polyoxyl 40 hydrogenated castor oil
Potassium acesulfame
Mint flavor
FD & C Red #40
D & C Red # 33

TABLE 12
Logl0 growth of organisms after exposure to antimicrobial compositions

| Microorganisms | Control | Scope | Gum | OCP-M |
|---|---|---|---|---|
| MRSA | 6 | 2.95 | 2.61 | 0 |
| S. aureus | 6 | 2.95 | 2.63 | 0 |
| C. albicans | 6 | 3.18 | 3 | 0 |
| P. aeruginosa | 6 | 5 | 1.83 | 0 |

### 18. EXAMPLE 14

Antimicrobial efficacy of the following alcohol-free hand sanitizers was tested:

### Hand Sanitizer Aqueous Foam (Syn-Botanica Hand Sanitizer Foam)

| Main ingredients | % w/w |
|---|---|
| Octanediol | 0.8 |
| Lactic acid | 0.2 |
| Alcohol | 10.0 |
| Lemon oil | 0.10 |
| Lemon grass oil | 0.1 |
| Orange oil | 0.1 |
| Thymol | 0.05 |
| Vanilla extract | 0.3 |
| Cosmocil (PHMB) | 1.0 (0.2) |
| Benzalkonium chloride | 0.13 |
| Benzoic acid | 0.2 |
| Phenyl ethanol | 0.3 |
| Other ingredients | % w/w |
| Water | 81.69 |
| Glycerin | 1.0 |
| Hydroxypropyl methyl | 0.03 |
| cellulose stearoxy ether | |
| (Sangelose) 90 L | |
| Solubilizer (PEG-40 hydrogenated | 1.0 |
| Castor oil, Trideceth 9, Water) | |
| Plantasol | 1.0 |
| 2-Methyl-1,3-propanediol | 0.75 |
| (Solidol MP-D) | |
| Ethoxy diglycol | 0.5 |
| Isododocane and isononanoate | 0.5 |
| Silicone KF-6043 | 0.25 |

### Aqueous Hand Sanitizer Lotion-2 (Syn-Botanica Hand Sanitizer Lotion)

| Main ingredients | % w/w |
|---|---|
| Octanediol | 0.8 |
| Lactic acid | 0.2 |
| Alcohol | 10.0 |
| Lemon oil | 0.10 |
| Lemon grass oil | 0.1 |
| Orange oil | 0.1 |
| Thymol | 0.05 |
| Vanilla extract | 0.3 |
| Cosmocil (PHMB) | 1.0 (0.2) |
| Benzalkonium chloride | 0.13 |
| Benzoic acid | 0.2 |
| Phenyl ethanol | 0.3 |
| Other ingredients | |
| Water | 69.77 |
| Oat powder | 1.0 |
| Hydroxypropyl methyl-cellulose | 0.2 |
| stearoxy ether (Sangelose) | |
| Glycerin | 4.0 |
| Incroquat Behenyl TMS | 2.0 |
| Polowax NF | 1.0 |
| Stearyl alcohol | 0.8 |
| Zinc oxide | 0.6 |
| Silicone fluid (Dimethicone200/350) | 2.0 |
| Almond oil | 2.0 |
| Lactic acid | 0.3 |
| Aloe juice | 2.0 |
| Bisabolol | 0.05 |
| Zemea | 1.0 |
| Adjust pH to 4.0-4.5 | |

### 18.1 Test 1

| | | | (Log₁₀ | (Log₁₀ |
|---|---|---|---|---|
| | | | Reduction | Reduction |
| | | (Log₁₀ | in 15 | in 1 minute) |
| | (Log₁₀ | Reduction | seconds) | % Kill |
| | Reduction in | in 15 | % Kill | Syn- |
| | 15 seconds) % | seconds) | Syn-Botanica | Botanica |
| | Kill | % Kill | Hand | Hand |
| | Soap: 0.3% | Soap: 0.3% | Sanitizer | Sanitizer |
| Organisms | Triclosan | PCMX | Foam | Lotion |
| S. aureus | (3.2) > 99.9 | (2.7) > 99 | (3.0) > 99.9 | (3.5) > 99.9 |
| (6538) ** | | | | |
| P. aeruginosa | (2.0) > 99 | (2.8) > 99 | (6.1) > 99.9 | (6.1) > 99.9 |
| (15442) | | | | |
| E. coli (35218) | (2.5) > 99 | (2.8) > 99 | (6.2) > 99.9 | (5.2) > 99.9 |
| MRSA (4716) | (2.6) > 99 | (2.6) > 99 | (3.3) > 99.9 | (5.0) > 99.9 |
| S. epidermidis | (3.6) > 99.9 | (1.4) < 99 | (3.5) > 99.9 | (4.5) > 99.9 |
| (35983) | | | | |
| E. faecalis | (3.2) > 99.9 | (2.98) > 99 | (3.5) > 99.9 | (4.0) > 99.9 |
| (29212) | | | | |
| VREF(Clinical | (3.57) > 99.9 | (3.3) > 99.9 | (3.0) > 99.9 | (3.5) > 99.9 |
| isolate) | | | | |
| S. marcescens | (0.60) < 90 | (1.0) < 99 | (8.3) > 99.9 | (6.0) > 99.9 |
| (14756) | | | | |
| A. baumanni | (4.86) > 99.99 | (2.85) > 99 | (3.0) > 99.9 | (3.5) > 99.9 |
| (Clinical | | | | |
| isolate) | | | | |
| E. aerogenes | (1.56) < 99 | (1.0) < 99 | (3.7) > 99.9 | (3.0) > 99.9 |
| (14053) | | | | |
| K. pneumonia | (1.95) < 99 | (2.8) > 99 | (8.0) > 99.99 | (6.0) > 99.9 |
| (13883) | | | | |
| S. typhimurium | (0.2) < 90 | (1.9) < 90 | (3.5) > 99.9 | (3.5) > 99.9 |
| (14028) | | | | |
| C. albicans | (0.1) < 90 | (0.4) < 90 | (6.2) > 99.9 | (3.5) > 99.9 |
| (10231) | | | | |

Rapid (15 second) and broad spectrum antibacterial efficacy against various microorganisms were tested. ASTM E 2783-11 method for testing the antimicrobial efficacy using a time kill procedure was used. The rapid antibacterial activity (15 seconds exposure) of the hand foam and lotion was tested against various organisms listed in Table 11.

### Table 11: Rapid and Broad Spectrum Antibacterial Efficacy of Triclosan, PCMX Soaps and Syn-Botanica Hand Sanitizers

ATCC=American Type Culture Collection; MRSA=Methicillin-resistant S. aureus; VREF=Vancomycin-resistant E. faecalis. The control growth ranged from 5×10⁵ to 1×106 cfu/ml.
Conclusion: Syn-Botanica hand sanitizer foam and lotion exhibit superior antimicrobial efficacy than Triclosan/PCMX hand cleansers.

### 18.2 Test 2

Rapid and Persistent Antibacterial Efficacy of Syn-Botanica Hand Sanitizer by Pigskin Method which Simulates the Volunteer Method of the American Society for Testing and Materials ASTM E2755-10 (Test Organism: S. aureus)

Method: 3 cm² pieces of pigskins were prepared and each piece was mounted on a petridish which simulates the band surface. Pigskins were contaminated with bacteria and rinsed with drug inactivating media. The colony counts in the rinse was determined by subculturing on agar plates and incubating for 24 hours at 37° C. (Baseline Counts). The same pair was then inoculated with bacteria and treated with the sanitizer and the bacterial counts were determined as before. The skin was again contaminated with bacteria and treated with the sanitizer and bacterial counts were determined as described above. Contamination and antibacterial product application were repeated 10 times. The bacterial counts were determined after the first and tenth applications. The reduction in bacterial counts after the first and tenth repeated cycles of contamination and product application, were compared to that of the Baseline Counts. Results are shown in the table below:

Test organisms S. aureus (ATCC 6538)
Log₁₀ reduction from control growth, mean ± SD

| Test Soap | S. aureus |
|---|---|
| Soap: 0.3% Triclosan¹ | |
| After 1^{st} application | 2.15 ± 0.09 |
| After 10^{th} application | 2.30 ± 0.06 |
| Alcohol hand sanitizer (Purell) | |
| After 1^{st} application | 2.1 ± 0.08 |
| After 10^{th} application | 1.9 ± 0.05 |
| Syn-Botanica hand sanitizerFoam | |
| After 1^{st} application | 2.0 ± 0.06 |
| After 10^{th} application | 2.5 ± 0.05 |
| Syn-Botanica hand sanitizer lotion | |
| After 1^{st} application | 2.1 ± 0.06 |
| After 10^{th} application | 3.0 ± 0.05 |

Control counts ranged from 1 × 10⁵ to 5 × 10⁵ cfu/ml. ¹Gojo soap containing 0.3% triclosan (T)
Conclusion: As per US FDA-TFM criteria the required log₁₀ reduction from control counts is 2.0 log after first application [rapid] and 3.0 log after tenth application [persistent] activity. Syn Botanica products show the required log reduction.
Conclusion: Syn-Botanica can be used in personal care products as an effective sanitizer with a low of skin sensitization. This can be also used for surface disinfection.

### 18.3 Test 3

Anti-inflammatory, anti itch and anti microbial topical cream/gel (SB AI-3) was tested. Evaluation of efficacy in human volunteers was determined.

### SynBotanica Anti-Inflammatory, Anti-Irritant Topical Gel “SB AI-3”:

| Ingredients | % w/w |
|---|---|
| Main ingredients | |
| PHMB | 0.6 |
| Lemon/citrus extract | 0.15 |
| Cinnamon oil | 0.05 |
| Rosemary oil | 0.1 |
| Octanediol | 0.8 |
| Oat Powder | 2.0 |
| Tetrahydrocurcumanoid | 0.1 |
| Other ingredients | |
| Water | 15.45 |
| Lactic acid | 0.1 |
| Natural Petrolactum | 40 |
| Aloe Gel | 30.0 |
| Pomegranate oil | 0.3 |
| Portulaca extract | 0.75 |
| Lemon grass oil | 0.1 |
| Tocopherol | 0.2 |
| Camphor | 0.3 |
| Dracocorin (Natural emulsifier) | 2.0 |

18.3.1 Anti-inflammatory Evaluation:
Prevention of blister formation and redness after minor burn

| Test | Volunteers | Before application | After application* |
|---|---|---|---|
| SB AI-3 Gel | 1 | Redness 3+ | 1+ |
| | 2 | +++ | — |
| | 3 | +++ | — |

18.3.2 Antitich Evaluation
Prevention of itching after mosquito bite

| Test | Volunteers | Before application | After application* |
|---|---|---|---|
| SB AI-3 Gel | 1 | +++ | — |
| | 2 | +++ | — |
| | 3 | +++ | — |

18.3.3 Prevention of Prickly heat Itch

| Test | Volunteers | Before application | After application* |
|---|---|---|---|
| SB AI-3 Gel | 1 | +++ | — |
| | 2 | +++ | — |
| | 3 | +++ | — |

18.3.4 Prevention of dry skin itch

| Test | Volunteers | Before application | After application* |
|---|---|---|---|
| SB AI-3 Gel | 1 | +++ | — |
| | 2 | +++ | — |
| | 3 | +++ | — |

18.3.5 Antimicrobial Evaluation

| | Zone of Inhibition (mm) | | |
|---|---|---|---|
| Test | P. aeuroginosa | S. aureus | C. albicans |
| SBAI-3 Gel | 8.0 | 12.5 | 18 |
| 2% miconazole | 8.0 | 13 | 15 |
| cream | | | |

*10 minutes after application.

### 19. EXAMPLE 15

### Alcohol-Free Botanical Oral Care Rinse Effective Against Strep. Mutans as Well as Pathogens Responsible for Ventilator Associated Pneumonia

A novel botanical oral rinse (BOR-1) containing a synergistic combination of antimicrobial botanicals as well as anti inflammatory botanicals along with a non irritant preservative (PHMB) was developed. As described below, BOR-1 was found to be effective against plaque forming S. mutants. This oral rinse is also effective against ventilator associated pneumonia (VAP) causing pathogens. Evaluation of 0.12% chlorhexidine (CHX) solutions as a prophylactic prior to mechanical intubation has concluded that oral decontamination can reduce the risk of acquiring VAP. Although CHX has been used as an oral antiseptic, adverse effects, such as irritation of the oral mucosa, discoloration of teeth and altered taste perception have been reported with its use. (Gúrgan C A, Zaim E, Bakirsoy I, Soykan E. Short-Term Side Effects of 0.2% Alcohol-Free Chlorhexidine Mouth rinse Used as an Adjunct to Non-Surgical Periodontal Treatment: A Double-Blind Clinical Study. J Periodontol 2006; 77:370-384). Moreover, studies have indicated that CHX is cytotoxic to murine fibroblasts cell lines and to human dennal, gingival and osteoblastic cell lines (Lee T H, Hu C C, Lee S S, Chou M Y, Change Y C. Cytotoxicity of chlorhexidine on human osteoblastic cells is related to intracellular glutathione levels. Int Endod J 2010; 43:430-435). As described below, the rapid antibacterial activity of the novel oral rinse BOR-1, chlorhexidine-containing oral rinse (PerioRx), and Listerine was evaluated and compared.

BOR-1 displayed better rapid antimicrobial efficacy to that of the chlorhexidine-containing rinse especially against S. aureus MRSA, and C. albicans. BOR-1 when exposed for 24 hours to tightly adhered (grown for 4 hours on agar plate) organisms on agar plates showed similar efficacy to Chlorhexidine oral rinse. Listerine was not as effective. When these oral rinses were exposed for 1 minute to various organisms grown on Agar plates for 4 hours, only BOR was effective against all the organisms tested. In contrast, Chlorhexidine oral rinse was effective only against C. albicans. This botanical composition can also be incorporated in Toothpaste/gel and dental floss.

Symbotanica oral rinse (BOR-1)

| Ingredients | % w/w |
|---|---|
| Lemon extract | 0.6 |
| Synthetic AB X | 0.12 |
| Cinnamon bark oil | 0.07 |
| Lemon grass oil | 0.07 |
| Basil oil | 0.07 |
| Clove bud oil | 0.03 |
| Peppermint oil | 0.04 |
| Hydrolite 5 | 0.5 |
| Solubilizer (Caprylyl capryl glucoside) | 2.0 |
| Solubilizer (Polyethylene glycol-40 | 0.5 |
| Hydrogenated castor oil) | |
| Water | 80.20 |
| Xylitol | 0.25 |
| Sorbitol | 0.25 |
| Pluronic F087 | 0.2 |
| Citric acid | 0.2 |
| Zinc salicylate | 0.1 |
| Propylene glycol | 5.0 |
| Glycerine | 5.0 |
| Aloe leaf juice | 1.0 |

“Synthetic AB X” is polyhexamethylene biguanide (PHMB)

### 19.1 Rapid and Broad Spectrum Antimicrobial Activity In-Vitro Rapid (15 Second Contact) Kill Method

### 19.1.1 Method

To test the rapid antimicrobial efficacy of the mouth rinses against various bacteria, ASTM standard E 2783-11 method was used. Briefly, 0.1 ml of bacterial culture (107 cfu/ml) was added to 0.9 ml of test mouthwash, BOR-1 or control samples and mixed for 15 seconds. The reaction was stopped with drug neutralizing Fluid (DNF) and then serially diluted with the same media. 0.5 ml of the dilutions was then plated on Trypticase soy plate and incubated at 37° C. for 24-48 hours. The colony counts were determined and the log 10 reduction from the control (phosphate-buffered saline (PBS) inoculated with the same culture) was calculated.

### 19.1.2 Results

### Rapid and Broad Spectrum Antimicrobial Activity of Oral Rinses (Log 10 Growth of Organisms Exposed to Antimicrobial Composition)

| | | | PerioRx | |
|---|---|---|---|---|
| Microorganisms | Control | Listerine | (CHx) | BOR-1 |
| S. aureus | 7.66 | 4.10 | 5.70 | 1.90 |
| MRSA | 7.05 | 4.39 | 4.75 | 1.23 |
| S. nutans | 7.33 | 1.33 | 1.33 | 1.33 |
| C. albicans | 6.00 | 3.37 | 4.67 | 1.68 |
| P. aeruginosa | 5.57 | 1.61 | 0.97 | 0.56 |

### 19.1.3 Conclusion

BOR-1, Listerine and PerioRx (Chlorhexidine (0.12%)) are effective in rapidly inactivating Strept. mutans after a 15 second contact time. BOR-1 was more effective against all other pathogens.

### 19.2 Antimicrobial Efficacy of Oral Rinses after Prolonged Contact with Colonized Bacteria on Agar Plates

### 19.2.1 Method

For this test, 0.5 ml of microbial cultures of 10⁴ cfu/ml were seeded on Trypticase Soy agar Plates (TSA) and incubated for 4 hours at 37° C. in order for the pathogens to tightly adhere and colonize the agar surface. 0.5 ml of oral rinses, and PBS (for the control sample) was then added and spread to cover the surface of the plates. The plates were then incubated at 37° C. for 24 hours. The colony counts on the plate were enumerated.

### 19.2.2 Results

### Antimicrobial Efficacy of Oral Rinses after Prolonged Exposure to Bacteria (Log 10 Growth of Organisms Exposed to Antimicrobial Composition)

| Microorganisms | Control | Listerine | PerioRx (CHx) | BOR-1 |
|---|---|---|---|---|
| S. aureus | 3.56 | 2.83 | 0.2 | 0.2 |
| MRSA | 3.62 | 2.59 | 0.2 | 0.2 |
| C. albicans | 3.19 | 0 | 0.2 | 0.2 |
| P. aeruginosa | 3.45 | 3.44 | 0.1 | 0.1 |

### 19.2.3 Conclusion

Chlorhexidine, BOR-1 products were effective in killing colonized pathogens. Listerine was not effective on colonized pathogens except for C. albicans

### 19.3 Efficacy of Oral Rinse that has been Coated on Agar Plates and Dried for 1 Hour in Inactivating Organisms

### 19.3.1 Method

In this method, 0.5 ml of oral rinses, or PBS (for the control sample) were first spread on TSA and incubated for 1 hour at 37° C. 0.3 ml×10³ cfu/ml microbial cultures were then spread onto the plates, which were then incubated at 37° C. overnight. The colony counts were enumerated.

### 19.3.2 Results

### Antibacterial Activity of Oral Rinses Coated and Dried for 1 Hour on Agar Plates (Log 10 Growth of Organisms Exposed to Antimicrobial Composition)

| | | | PerioRx | BOR- |
|---|---|---|---|---|
| Microorganisms | Control | Listerine | (CHx) | 1 |
| S. aureus | 3.56 | 2.83 | 0.1 | 0.1 |
| MRSA | 3.62 | 2.59 | 0.1 | 0.1 |
| C. albicans | 3.45 | 3 | 0.1 | 0.1 |
| P. aeruginosa | 3.45 | 1.76 | 0.1 | 0.1 |

### 19.3.3 Conclusion

Both Chlorhexidine and BOR-1 demonstrated excellent residual antimicrobial efficacy. Listerine was ineffective. This may be due to the evaporation of alcohol and the absence of antibacterial actives in the product to kill the pathogens

### 19.4 Antibacterial Activity of Oral Rinses on Bacteria Colonizing Cavities in Between Teeth

### 19.4.1 Method

12 well-shaped cavities (6 wells each on right and left side of a TSA plate) were created in each plate with a cork borer. 0.3 ml×10³ cfu/ml microbial cultures were then spread onto the plates and incubated for 1 hour. 0.5 ml of the oral rinses, and control (PBS) samples were spread on each plate. The colony counts on the surface as well as in the cavities were determined after overnight incubation. The colonies inside/underneath the cavities were distinguishable from colonies on the surface, because the penetrating colonies appeared superimposed to the colonies growing on the surface.

### 19.4.2 Results

### Antibacterial Activity of Oral Rinses on Bacteria Colonized on the Agar Surface as Well as in the Cavities

### 19.4.2.1 Surface Bacterial Counts

### Log 10 Growth of Organisms Exposed to Antimicrobial Composition

| | | | PerioRx | |
|---|---|---|---|---|
| Microorganisms | Control | Listerine | (CHx) | BOR-1 |
| S. aureus | 3.1 | 2.68 | 0.1 | 0.1 |
| MRSA | 3.1 | 2.74 | 1.38 | 0.1 |
| C. albicans | 2.99 | 1.63 | 1.34 | 0.1 |
| P. aeruginosa | 3.2 | 3.11 | 1.34 | 0.1 |

### 19.4.2.2 Penetrated Bacterial Counts

### Log 10 Growth of Organisms Exposed to Antimicrobial Composition

| | | | PerioRx | |
|---|---|---|---|---|
| Microorganisms | Control | Listerine | (CHx) | BOR-1 |
| S. aureus | 2.27 | 2.23 | 0.1 | 0.1 |
| MRSA | 2.39 | 2.28 | 0.1 | 0.1 |
| C. albicans | 2.1 | 1.34 | 1.94 | 0.1 |
| P. aeruginosa | 2.24 | 2.25 | 0.1 | 0.1 |

### 19.4.3 Conclusion

BOR-1 effectively killed both surface and penetrating pathogens for all the pathogens tested. Chlorhexidine rinse was less effective against C. albicans. Listerine was ineffective against all of the pathogens tested.

### 19.5 Antibacterial Activity of Oral Rinses after One Minute Contact with Colonized Bacteria on Agar Plates (S. Mutants)

### 19.5.1 Method

For this test, 0.5 ml of microbial cultures of S. mutant (108 cfu/ml) was seeded on Muller Hinton agar plate (MHA) and incubated for 5 days at 37° C. in order for the pathogens to tightly adhere and colonize the agar surface. On 5th day, 0.5 ml of oral rinses and PBS (for the control sample) were then added and spread onto various plates, After 1 minute, 1 ml drug inactivating media was added to the plate and swirled to cover the whole plate and kept for 5 minutes. All the bacterial colonies were collected from the plates using a liquid media and sub cultured on another plate and was then incubated at 37° C. for 48 hours. The colony counts on the plate were enumerated.

### 19.5.2 Results

### Antibacterial Activity of Oral Rinses on Bacteria Colonized on the Agar Plates (S. mutans) (Log 10 Growth of Organisms Exposed to Antimicrobial Composition)

Antibacterial activity of
oral rinses on
bacteria colonized
on the agar plates (S. nutans)

| Groups | Log₁₀ growth in cfu/ml |
|---|---|
| Control | 7.59 |
| Listerine | 7.49 |
| Scope | 7.26 |
| GUM | 7.56 |
| BOR-1 | 2.41 |

### 19.6 Summary

BOR-1 displayed superior, rapid antimicrobial efficacy when compared to that of the other oral rinses tested. The testing indicates that a 15 second rinse would eliminate loosely adhered bacteria in the oral cavity. Furthermore, when exposure times were longer (24 hours), BOR-1 showed similar or more antimicrobial activity against tightly adhered organisms than that of the chlorhexidine-containing rinse. BOR-1 may be potent and safer alternatives to Listerine or chlorhexidine containing oral rinses for general use as well as in the prophylaxis of ventilator-associated pneumonia.

### 20. EXAMPLE 16

The antimicrobial efficacy of a composition comprising biguanides and fennel oil, thyme oil/thymol and/or vanilla oil was tested against S. aureus (10⁷ cfu/ml). The compositions were tested to demonstrate that the antimicrobial activity of Enhancement of biguanides is enhanced by fennel oil, thyme oil/thymol and/or vanilla oil.

In-vitro study Test organism: S. aureus (10⁷ cfu/ml)

### Base a Composition:

| Ingredient | % w/w |
|---|---|
| Zemea | 4.0 |
| Propylene glycol | 2.0 |
| Cinnamon bark oil | 0.01 |
| Basil oil | 0.01 |
| Peppermint oil | 0.05 |
| Plantasol | 0.5 |
| Water | 71.0 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.25 |

The ingredients listed in the following experiments 1 and 2 were incorporated into Base A and tested for antimicrobial efficacy using the method described below:

### Method

### ASTM E 2783-11 Protocol

Bacterial cultures (S. aureus) were prepared as described in the ASTM E 2783-11 protocol. 0.1 ml of 10⁷ cfu/ml of bacterial culture was suspended in sterile saline placed in a sterile culture tube.
0.9 ml of the tested oral rinse formulation was added to the tube and vortexed for 15 seconds [Phosphate buffered saline (PBS) was used instead of the tested oral rinse for the control group].
9.0 ml Drug Neutralizing Fluid (DNF) was added to the tube (1:10 dilution) to neutralize the activity of the tested oral rinse; this tube was then vortexed and serially diluted to facilitate colony counting.
0.5 ml of the last dilution was plated on trypticase soy agar plates (TSA). These plates were incubated at 37° C. for 24 hours and the colony counts (cfu)/ml were determined.

### Experiment 1:

| Tested Ingredients | Concentration | Log reduction |
|---|---|---|
| control | (% w/w) | compared to |
| T (Thymol) | 0.1 | 1.29 |
| B (Basil oil) | 0.1 | 0.84 |
| Chlorhexidine (CHG) | 0.12 | 0.73 |
| CHG + T | 0.12 + 0.1 | 2.44 |
| CHG + B | 0.12 + 0.1 | 2.03 |
| F (Fennel seed oil) + T | 0.1 + 0.1 | 1.65 |
| F + T + V (Vanilla oil) | 0.1 + 0.1 + 0.1 | 1.95 |
| CHG + F + T | 0.12 + 0.1 + 0.1 | 3.05 |
| CHG +F + T +V | 0.12 + 0.1 + 0.1 + 0.1 | 3.26 |

The volume of all the above compositions after incorporation in Base A was adjusted to 100% with water.

### Conclusion:

Chlorhexidine exhibits synergistic activity when combined with either thymol or basil oil. Furthermore, the combination of either fennel seed oil and thymol or fennel seed oil, thymol and vanilla oil with chlorhexidine also exhibited synergistic activity.

### Experiment 2:

| Tested Ingredients | Concentration | Log reduction |
|---|---|---|
| control | (% w/w) | compared to |
| T (Thymol) | 0.1 | 1.29 |
| V (Vanilla oil) | 0.1 | 0.7 |
| PHMB | 0.1 | 1.96 |
| PHMB +T | 0.1 + 0.1 | 5.6 |
| PHMB +V | 0.1 + 0.1 | 3.86 |
| F (Fennel seed oil) +T | 0.1 + 0.1 | 1.65 |
| F +T +V (Vanilla oil) | 0.1 + 0.1 + 0.1 | 1.95 |
| PHMB +F +T | 0.1 + 0.1 + 0.1 | 4.64 |
| PHMB +F+T +V | 0.1 + 0.1 + 0.1 + 0.1 | 5.63 |

The volume of all the above compositions after incorporation in Base A was adjusted to 100% with water.

### Conclusion:

PHMB exhibited synergistic activity when combined with either thymol or vanilla oil. Furthermore, the combination of either fennel seed oil and thymol or fennel seed oil, thymol and vanilla oil with PHMB also exhibited synergistic activity.

### 21. EXAMPLE 17

### Evaluation of Oral Care Compositions PTFV1 and CTFV

Plate Method: Rapid Anti-Microbial Efficacy of Oral Rinses Exposed to 4 Hour Incubated Culture (S. aureus):

0.3 ml of S. aureus culture (10⁵ cfu/mL) or Candida albicans (10⁵ cfu/ml) was seeded on TSA plates and incubated for 4 hours at 37° C. in such that the order microorganisms adhered tightly to the plates and colonized the surface of the plates. 0.5 ml of test oral rinses or PBS (control) was then added and spread on the surface of the plates. After 1 minute, 2.5 mL of Drug Neutralizing Fluid (DNF) was added, swirled to cover whole plate, and transferred into sterile culture tubes. 2 ml of DNF was added to rinse the plate thoroughly and transferred into the same culture tube. The tube was vortexed and serially diluted to facilitate colony counting. 0.5 ml of the last dilution was plated on trypticase soy agar plates (TSA), which were incubated at 37° C. for 24 hours and the colony counts (cfu)/ml were determined.

Experiment 1A: Test organism: S. aureus (10⁵ cfu/ml)

| Test Group | Growth | Log reduction |
|---|---|---|
| control | (log₁₀) | compared to |
| PBS (Control) | 6.3 | |
| Listerine | 4.2 | 2.1 |
| 0.12% CHG (GUM) | 3.47 | 2.83 |
| PTFV1 Oral Rinse | 1.0 | 5.3 |
| PTFV- E1 Oral Rinse | 0.0 | 6.3 |
| CTFV Oral Rinse | 2.73 | 3.57 |
| CTFV-E Oral Rinse | 1.33 | 4.97 |

### Conclusion:

Both of the PTFV and CTFV oral rinses exhibited higher anti-bacterial efficacy against S. aureus compared to control. Furthermore, addition of vanilla extract (0.3%) to both of the PTFV and CTFV oral rinses (PTFV-E and CTFV-E) enhanced the anti-bacterial efficacy against S. aureus.

Experiment 2A: Test Organism: Candida albicans (10⁵ cfu/ml)

| Group | Growth | Log reduction |
|---|---|---|
| control | (log₁₀) | compared to |
| PBS (Control) | 5.93 | |
| Listerine | 2.78 | 3.15 |
| 0.12% CHG (GUM) | 3.31 | 2.62 |
| PTFV1 Oral Rinse | 2.79 | 3.14 |
| CTFV Oral Rinse | 2.69 | 3.24 |
| CTFV -E Oral Rinse | 2.47 | 3.46 |

### Conclusion:

Both of the PTFV and CTFV oral rinses exhibited higher anti-bacterial efficacy against Candida albicans compared to control. Furthermore, addition of vanilla extract (0.3%) to the CTFV oral rinse (CTFV-E) enhanced the anti-bacterial efficacy against Candida albicans.

Various patents and publications are cited herein, the contents of which are hereby incorporated by reference in their entireties herein.

We claim:

1. An antimicrobial composition comprising:
(a) one or more plant extract selected from the group consisting of vanilla extract, cedarwood extract, lemon extract, orange extract, grapefruit seed extract, Bacopa monniera extract, honeysuckle extract, Echinacea extract, goldenseal (Hydrastis canadensis) extract, Portulaca oleracea extract, witchhazel extract, rosemary extract, stevia extract, tobacco extract, wasabi extract, willow bark extract, and combinations thereof, at a concentration of between about 0.1 and 1.0% w/w or between about 0.0 and 5.0% w/w; and
(b) one or more synthetic antimicrobial selected from the group consisting of biguanide, quaternary ammonium compound, hypochlorite, hydrogen peroxide, urea hydrogen peroxide, and combinations thereof, at a concentration of between about 0.001 and 0.5% w/w or between about 0.001 and 5.0% w/w;
(c) essentially no benzyl alcohol and less than 21% w/w total alcohol;
(d) one or more alkanediol selected from the group consisting of propanediol, pentanediol, hexanediol, heptanediol, octanediol, nonanediol, decandediol, and combinations thereof, at a concentration of between about 0.0 and 2.0% w/w or between about 0.0 and 10.0% w/w;
(e) a solubulizer;
(f) one or more organic acid selected from the group consisting of lactic acid, citric acid, benzoic acid, and combinations thereof, at a concentration of between about 0.1 and 0.5% w/w or between about 0.01 and 20.0% w/w; and
(g) one or more essential oil or constituent thereof selected from the group consisting of thyme oil, thymol, rosemary oil, lemongrass oil, lemon oil, orange oil, vanilla oil, fennel seed oil, cinnamon oil, cinnamaldehyde, cinnamylacetic ester, cinnamic acid, ethyl cinnamate, fennel oil, basil oil, clove oil, pine oil, cedarwood oil, farnesol, bisabalol, and combinations thereof, at a concentration of between about 0.05 and 0.5% w/w or between about 0.01 and 5.0% w/w.

2. The composition of claim 1, where the plant extract comprises lemon extract or vanilla extract.

3. The composition of claim 1, further comprising a botanical anti-irritant selected from the group consisting of aloe gel or extract at a concentration between about 0.1-50% w/w, aloe leaf juice at a concentration between about 0.5-2.05% w/w, bisabalol at a concentration between about 0.05-2.0% w/w, an oat compound at a concentration between about 0.2-3.0% w/w, and combinations thereof.

4. The composition of claim 1, further comprising an anti-inflammatory agent selected from the group consisting of calendula oil, calendula extract, portulaca extract, rosemary oil, a curcumin compound, pomegranate oil, Echinacea purpurea extract, resveratrol, witchhazel extract, and combinations thereof.

5. An antimicrobial composition comprising:
(a) one or more plant extract selected from the group consisting of vanilla extract, lemon extract, orange extract, grapefruit seed extract, Bacopa monniera extract, honeysuckle extract, Echinacea extract, goldenseal (Hydrastis canadensis) extract, Portulaca oleracea extract, witchhazel extract, rosemary extract, stevia extract, tobacco extract, wasabi extract, willow bark extract, and combinations thereof, at a concentration of between about 0.1 and 1.0% w/w;
(b) one or more synthetic antimicrobial selected from the group consisting of biguanide, quaternary ammonium compound, and combinations thereof, at a concentration of between about 0.001 and 0.5% w/w;
(c) essential oil or constituent thereof selected from the group consisting of thyme oil, thymol, cinnamon oil, cinnamaldehyde, cinnamylacetic ester, cinnamic acid, ethyl cinnamate, rosemary oil, lemongrass oil, lemon oil, orange oil, cinnamon oil, fennel oil, basil oil, clove oil, farnesol, bisabalol, pine oil, cedarwood oil, and a combination thereof, at a concentration of between about 0.01 and 0.5% w/w; and
(d) essentially no benzyl alcohol.

6. The composition of claim 5, further comprising between about 5 and 50% w/w alcohol.

7. The composition of claim 5, further comprising an organic acid selected from the group consisting of lactic acid, citric acid, benzoic acid, and combinations thereof, at a concentration of between about 0.05 and 5% w/w.

8. The composition of claim 5, further comprising an organic acid selected from the group consisting of lactic acid, citric acid, and combinations thereof, at a concentration of between about 5 and 20% w/vv.

9. The composition of claim 5, further comprising a non-ionic surfactant or cationic surfactant at a concentration of between about 5 and 20% w/w.

10. The composition of claim 5, wherein the plant extract comprises lemon extract, vanilla extract, or combinations thereof.

11. The composition of claim 5, wherein the synthetic antimicrobial comprises a biguanide selected from the group consisting of polyhexamethylene biguanide, polyaminopropyl biguanide, and combinations thereof.

12. The composition of claim 5, further comprising an alkanediol selected from the group consisting of propanediol, pentanediol, hexanediol, heptanediol, octanediol, nonanediol, decandediol, and combinations thereof, at a concentration between about 0.0 and 2.0.% w/w.

13. The composition of claim 5, further comprising a solvent selected from the group consisting of alcohol at a concentration between about 0 and 80% w/w; phenyl ethanol at a concentration between about 0 and 1% w/w; a mixture of PEG-40 hydrogenated castor oil, trideceth-9, propylene glycol and water at a concentration between about 0 and 2% w/w; a mixture of PEG-40 hydrogenated castor oil, trideceth-9 and water at a concentration between about 0 and 2% w/w; caprylyl capryl glucoside (Plantasol) at a concentration between about 0 and 2% w/w; and combinations thereof.

14. The composition of claim 5, further comprising an anti-irritant selected from the group consisting of zinc salicylate, basil oil, brahmi extract, red sandal wood extract, curcumin compound, aloe, bisabolol, rosemary oil, chalmogra oil, oat powder, oat extract, beta glucan, chitosan, calendula oil, portulaca extract, witchhazel extract, pomegranate oil, and combinations thereof, at a concentration between about 0.1 and 2.0% w/w.

15. The composition of claim 5, where the essential oil or constituent thereof is selected from the group consisting of thyme oil, thymol, cinnamon oil, cinnamaldehyde, cinnamylacetic ester, cinnamic acid, ethyl cinnamate, basil oil, fennel seed oil, and combinations thereof.

16. An antimicrobial composition comprising:
(a) one or more plant extract selected from the group consisting of vanilla extract, lemon extract, and combinations thereof, at a concentration of between about 0.1 and 1% w/w;
(b) polyhexamethylene biguanide (PHMB) at a concentration of between about 0.001 and 0.5% w/w; and
(c) an essential oil or constituent thereof selected from the group consisting of thyme oil, thymol, cinnamon oil, cinnamaldehyde, cinnamylacetic ester, cinnamic acid, ethyl cinnamate, basil oil, fennel seed oil, and combinations thereof, at a concentration of between about 0.01 and 0.5% w/w;
wherein the composition comprises essentially no benzyl alcohol.

17. An antimicrobial composition comprising:
(a) one or more essential oil or constituent thereof selected from the group consisting of thyme oil, thymol, fennel oil, vanilla oil, basil oil and combinations thereof, at a concentration of between about 0.01 and 0.5% w/w; and
(b) one or more synthetic antimicrobial selected from the group consisting of a biguanide, a quaternary ammonium compound, hydrogen peroxide, and combinations thereof,
wherein the biguanide, when present, is selected from the group consisting of chlorhexidine free base, chlorhexidine gluconate, chlorhexidine acetate, polyhexamethylene biguanide (PHMB), polyaminopropyl biguanide (PAPB), and combinations thereof, at a concentration of between about 0.01 and 0.5% w/w, and
wherein the quaternary ammonium compound, when present, is selected from the group consisting of benzalkonium chloride (BAC), benzethonium chloride (BZT), cetyl pyridinium chloride, alkyl dimethyl benzyl ammonium chloride, and combinations thereof, at a concentration of between about 0.02 and 0.5% w/w.

18. The antimicrobial composition of claim 17, wherein the composition comprises essentially no benzyl alcohol.

19. The antimicrobial composition of claim 17, wherein the synthetic antimicrobial comprises chlorhexidine gluconate.

20. The antimicrobial composition of claim 17, wherein the one or more essential oil or constituent thereof comprises thymol, and the one or more synthetic antimicrobial comprises chlorhexidine gluconate.

21. The antimicrobial composition of claim 17, wherein the one or more essential oil or constituent thereof comprises basil oil, and the one or more synthetic antimicrobial comprises chlorhexidine gluconate.

22. The antimicrobial composition of claim 17, wherein the one or more essential oil or constituent thereof comprises fennel oil and thymol; and the one or more synthetic antimicrobial comprises chlorhexidine gluconate.

23. The antimicrobial composition of claim 17, wherein the one or more essential oil or constituent thereof comprises fennel oil, thymol and vanilla oil; and the one or more synthetic antimicrobial comprises chlorhexidine gluconate.

24. The antimicrobial composition of claim 23, further comprising vanilla extract at a concentration of between about 0.1 and 0.6% w/w.

25. The antimicrobial composition of claim 17, wherein the synthetic antimicrobial comprises polyhexamethylene biguanide.

26. The antimicrobial composition of claim 17, wherein the one or more essential oil or constituent thereof comprises thymol, and the one or more synthetic antimicrobial comprises polyhexamethylene biguanide.

27. The antimicrobial composition of claim 17, wherein the one or more essential oil or constituent thereof comprises vanilla oil, and the one or more synthetic antimicrobial comprises polyhexamethylene biguanide.

28. The antimicrobial composition of claim 17, wherein the one or more essential oil or constituent thereof comprises fennel oil and thymol; and the one or more synthetic antimicrobial comprises polyhexamethylene biguanide.

29. The antimicrobial composition of claim 17, wherein the one or more essential oil or constituent thereof comprises fennel oil, thymol and vanilla oil; and the one or more synthetic antimicrobial comprises polyhexamethylene biguanide.

30. The antimicrobial composition of claim 29, further comprising vanilla extract at a concentration of between about 0.1 and 0.6% w/w.
